(19) United States
(12) Patent Application Publication
(10) Pub. No.: US 2004/0248251 A1
(43) Pub. Date: Dec. 9, 2004

(54) Receptors and membrane associated proteins

(76) Inventors: Preeti G Lal, Santa Clara, CA (US); Cynthia D Honchell, San Francisco, CA (US); Ian J Forsythe, Edmonton, CA (US); Narinder K Chawla, Union City, CA (US); Y Tom Tang, San Jose, CA (US); Mark L Borowsky, Northampton, MA (US); Ines Barroso, Cambridge (GB); Henry Yue, Sunnyvale, CA (US); Bridget A Warren, San Marcos, CA (US); Kavitha Thangavelu, Sunnyvale, CA (US); Kimberly J Gietzen, San Jose, CA (US); Yalda Azimzai, Oakland, CA (US); Ernestine A Lee, Kensington, CA (US); Mariah R Baughn, Los Angeles, CA (US); Ann E Gorvad, Bellingham, WA (US); Brendan M Duggan, Sunnyvale, CA (US); Bao Tran, Santa Clara, CA (US); Joana X Li, Millbrae, CA (US); Thomas W Richardson, Redwood City, CA (US); Vicki S Elliott, San Jose, CA (US); Yeganeh Zebarjadian, San Francisco, CA (US); Uyen K Tran, San Jose, CA (US); Monique G Yao, Mountain View, CA (US); David P Peterson, San Jose, CA (US); Wen Luo, San Diego, CA (US); Lehr-Mason Patricia, Morgan Hill, CA (US)

Correspondence Address: INCYTE CORPORATION, EXPERIMENTAL STATION, ROUTE 141 & HENRY CLAY ROAD, BLDG. E336, WILMINGTON, DE, 19880, US

(21) Appl. No.: 10/484,148

(22) PCT Filed: Jul. 16, 2002
(86) PCT No.: PCT/US02/22833

(30) Foreign Application Priority Data
Jul. 17, 2001 (US) 60306020
Jul. 27, 2001 (US) 60308179
Aug. 2, 2001 (US) 60309702
Aug. 10, 2001 (US) 60311476
Aug. 10, 2001 (US) 60311718
Aug. 10, 2001 (US) 60311551
Aug. 24, 2001 (US) 60314798
Aug. 31, 2001 (US) 60316639
Sep. 7, 2001 (US) 60317996

(51) Int. Cl. C07K 14/705; C07H 21/04

(52) U.S. Cl. 435/69.1; 435/320.1; 435/325; 530/350; 536/23.5

(57) ABSTRACT

Various embodiments of the invention provide human receptors and membrane-associated proteins (REMAP) and polynucleotides which identify and encode REMAP. Embodiments of the invention also provide expression vectors, host cells, anti-bodies, agonists, and antagonists. Other embodiments provide methods for diagnosing, treating, or preventing disorders associated with aberrant expression of REMAP.

### TECHNICAL FIELD

The invention relates to novel nucleic acids, receptors and membrane-associated proteins encoded by these nucleic acids, and to the use of these nucleic acids and proteins in the diagnosis, treatment, and prevention of cell proliferative, autoimmune/inflammatory, renal, neurological, cardiovascular, metabolic, developmental, endocrine, muscle, gastrointestinal, lipid metabolism, and transport disorders, and viral infections. The invention also relates to the assessment of the effects of. exogenous compounds on the expression of nucleic acids and receptors and membrane-associated proteins.

### BACKGROUND OF THE INVENTION

Signal transduction is the general process by which cells respond to extracellular signals. Signal transduction across the plasma membrane begins with the binding of a signal molecule, e.g., a hormone, neurotransmitter, or growth factor, to a cell membrane receptor. The receptor, thus activated, triggers an intracellular biochemical cascade that ends with the activation of an intracellular target molecule, such as a transcription factor. This process of signal transduction regulates all types of cell functions including cell proliferation, differentiation, and gene transcription.

Biological membranes surround organelles, vesicles, and the cell itself. Membranes are highly selective permeability barriers made up of lipid bilayer sheets composed of phosphoglycerides, fatty acids, cholesterol, phospholipids, glycolipids, proteoglycans, and proteins. Membranes contain ion pumps, ion channels, and specific receptors for external stimuli which transmit biochemical signals across the membranes. These membranes also contain second messenger proteins which interact with these pumps, channels, and receptors to amplify and regulate transmission of these signals.

### Plasma Membrane Proteins

Plasma membrane proteins (MPs) are divided into two groups based upon methods of protein extraction from the membrane. Extrinsic or peripheral membrane proteins can be released using extremes of ionic strength or pH, urea, or other disruptors of protein interactions. Intrinsic or integral membrane proteins are released only when the lipid bilayer of the membrane is dissolved by detergent.

Integral Membrane Proteins

The majority of known integral membrane proteins are transmembrane proteins (TM) which are characterized by an extracellular, a transmembrane, and an intracellular domain. TM domains are typically comprised of 15 to 25 hydrophobic amino acids which are predicted to adopt an α-helical conformation. TM proteins are classified as bitopic (Types I and II) and polytopic (Types III and IV) (Singer, S. J. (1990) Annu. Rev. Cell Biol. 6:247-96). Bitopic proteins span the membrane once while polytopic proteins contain multiple membrane-spanning segments. TM proteins that act as cell-surface receptor proteins involved in signal transduction include growth and differentiation factor receptors, and receptor-interacting proteins such as Drosophila pecanex and frizzled proteins, LIV-1 protein, NF2 protein, and GNS1/SUR4 eukaryotic integral membrane proteins. TM proteins also act as transporters of ions or metabolites, such as gap junction channels (connexins) and ion channels, and as cell anchoring proteins, such as lectins, integrins, and fibronectins. TM proteins act as vesicle organelle-forming molecules, such as calveolins, or as cell recognition molecules, such as cluster of differentiation (CD) antigens, glycoproteins, and mucins.

Many membrane proteins (MPs) contain amino acid sequence motifs that target these proteins to specific subcellular sites. Examples of these motifs include PDZ domains, KDEL, RGD, NGR, and GSL sequence motifs, von Willebrand factor A (vWFA) domains, and EGF-like domains. RGD, NGR, and GSL motif-containing peptides have been used as drug delivery agents in cancer treatments which target tumor vasculature (Arap, W. et al. (1998) Science, 279:377-380). Furthermore, MPs may also contain amino acid sequence motifs, such as the carbohydrate recognition domain (CRD), also known as the C-type lectin domain, that mediate interactions with extracellular or intracellular molecules.

Chemical modification of amino acid residue side chains alters the manner in which MPs interact with other molecules, for example, phospholipid membranes. Examples of such chemical modifications to amino acid residue side chains are covalent bond formation with glycosaminoglycans, oligosaccharides, phospholipids, acetyl and paimitoyl moieties, ADP-ribose, phosphate, and sulphate groups.

RNA encoding membrane proteins may have alternative splice sites which give rise to proteins encoded by the same gene but with different messenger RNA and amino acid sequences. Splice variant membrane proteins may interact with other ligand and protein isoforms.

Membrane proteins may also interact with and regulate the properties of the membrane lipids. Phospholipid scramblase, a type II plasma membrane protein, mediates calcium dependent movement of phospholipids (PL) between membrane leaflets. Calcium induced remodeling of plasma membrane PL plays a key role in expression of platelet anticoagulant activity and in clearance of injured or apoptotic cells (Zhou Q. et al. (1997) J. Biol. Chem. 272:18240-18244). Scott syndrome, a bleeding disorder, is caused by an inherited deficiency in plasma membrane PL scramblase function (Online Mendelian Inheritance in Man (OMIM) *262890 Platelet Receptor for Factor X, Deficiency of).

Tumor antigens are cell surface molecules that are differentially expressed in tumor cells relative to normal cells. Tumor antigens distinguish tumor cells immunologically from normal cells and provide diagnostic and therapeutic targets for human cancers (Takagi, S. et al. (1995) Int. J. Cancer 61: 706-715; Liu, E. et al. (1992) Oncogene 7: 1027-1032). One such protein is the neuron and testis specific protein Ma1, a marker for paraneoplastic neuronal disorders (Dalmau, J. et al. (1999) Brain 122:27-39).

Other types of cell surface antigens include those identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “CD” or “cluster of differentiation” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylnositol (GPI), discussed below. (Reviewed in Barclay, A. N. et al. (1995) The Leucocyte Antigen Facts Book, Academic Press, San Diego, Calif., pp. 17-20.)

The TM cell surface glycoprotein CD69 is an early activation antigen of T lymphocytes. CD69 is homologous to members of a supergene family of type II integral membrane proteins having C-type lectin domains. Although the precise functions of the CD-69 antigen is not known, evidence suggests that these proteins transmit mitogenic signals across the plasma membrane and are up-regulated in response to lymphocyte activation (Hamann, J. et. al. (1993) J. Immunol. 150:4920-4927).

Macrophages are involved in functions including clearance of senescent or apoptotic cells, cytokine production, hemopoiesis, bone resorption, antigen transport, and neuroendocrine regulation. These diverse roles are influenced by specialized macrophage plasma membrane proteins. The murine macrophage restricted C-type lectin is a type II integral membrane protein expressed exclusively in macrophages. The strong expression of this protein in bone marrow suggests a hemopoeitic function, while the lectin domain suggests it may be involved in cell-cell recognition (Balch, S. G. et al. (1998) J. Biol. Chem. 273:18656-18664).

Peripheral and Anchored Membrane Proteins

Some membrane proteins are not membrane-spanning but are attached to the plasma membrane via membrane anchors or interactions with integral membrane proteins. Membrane anchors are covalently joined to a protein post-translationally and include such moieties as prenyl, myristyl, and glycosylphosphatidyl inositol (GPI) groups. Membrane localization of peripheral and anchored proteins is important for their function in processes such as receptor-mediated signal transduction. For example, prenylation of Ras is required for its localization to the plasma membrane and for its normal and oncogenic functions in signal transduction.

The pancortins are a group of four glycoproteins which are predominantly expressed in the cerebral cortex of adult rodents. Immunological localization indicates that the pancortins are endoplasmic reticulum anchored proteins. The pancortins share a common sequence in the middle of their structure, but have alternative sequences at both ends due to differential promoter usage and alternative splicing. Each pancortin appears to be differentially expressed and may perform different functions in the brain (Nagano, T. et al. (1998) Mol. Brain Res. 53:13-23).

### Receptors

The term receptor describes proteins that specifically recognize other molecules. The category is broad and includes proteins with a variety of functions. The bulk of receptors are cell surface proteins which bind extracellular ligands and produce cellular responses in the areas of growth, differentiation, endocytosis, and immune response. Other receptors facilitate the selective transport of proteins out of the endoplasmic reticulum and localize enzymes to particular locations in the cell. The term may also be applied to proteins which act as receptors for ligands with known or unknown chemical composition and which interact with other cellular components. For example, the steroid hormone receptors bind to and regulate transcription of DNA.

Cell surface receptors are typically integral plasma membrane proteins. These receptors recognize hormones such as catecholamines; peptide hormones; growth and differentiation factors; small peptide factors such as thyrotropin-releasing hormone; galanin, somatostatin, and tachykinins; and circulatory system-borne signaling molecules. Cell surface receptors on immune system cells recognize antigens, antibodies, and major histocompatibility complex (MHC)-bound peptides. Other cell surface receptors bind ligands to be internalized by the cell. This receptor-mediated endocytosis functions in the uptake of low density lipoproteins (LDL), transferrin, glucose- or mannose-terminal glycoproteins, galactose-terminal glycoproteins, immunoglobulins, phosphovitellogenins, fibrin, proteinase-inhibitor complexes, plasminogen activators, and thrombospondin (Lodish, H. et al. (1995) Molecular Cell Biology, Scientific American Books, New York N.Y., p. 723; Mikhailenko, I. et al. (1997) J. Biol. Chem. 272:6784-6791).

Receptor Protein Kinases

Many growth factor receptors, including receptors for epidermal growth factor, platelet-derived growth factor, fibroblast growth factor, as well as the growth modulator α-thrombin, contain intrinsic protein kinase activities. When growth factor binds to the receptor, it triggers the autophosphorylation of a serine, threonine, or tyrosine residue on the receptor. These phosphorylated sites are recognition sites for the binding of other cytoplasmic signaling proteins. These proteins participate in signaling pathways that eventually link the initial receptor activation at the cell surface to the activation of a specific intracellular target molecule. In the case of tyrosine residue autophosphorylation, these signaling proteins contain a common domain referred to as a Src homology (SH) domain. SH2 domains and 8H3 domains are found in phospholipase C-γ, PI-3-K p85 regulatory subunit, Ras-GTPase activating protein, and pp60^{c-src} (Lowenstein, E. J. et al. (1992) Cell 70:431-442). The cytokine family of receptors share a different common binding domain and include transmembrane receptors for growth hormone (GH), interleultins, erythropoietin, and prolactin.

Other receptors and second messenger-binding proteins have intrinsic serine/threonine protein kinase activity. These include activin/TGF-β/BMT-superfamily receptors, calcium- and diacylglycerol-activated/phospholipid-dependant protein kcinase (PK-C), and RNA-dependant protein kinase (PK-R). In addition, other serine/threonine protein kinases, including nematode Twitchin, have fibronectin-like, immunoglobulin C2-like domains.

G-Protein Coupled Receptors

The G-protein coupled receptors (GPCRs), encoded by one of the largest families of genes yet identified, play a central role in the transduction of extracellular signals across the plasma membrane. GPCRs have a proven history of being successful therapeutic targets.

GPCRs are integral membrane proteins characterized by the presence of seven hydrophobic transmembrane domains which together form it bundle of antiparallel alpha (α) helices. GPCRs range in size from under 400 to over 1000 amino acids (Strosberg, A. D. (1991) Eur. J. Biochem. 196:1-10; Coughin, S. R. (1994) Curr. Opin. Cell Biol. 6:191-197). The amino-terminus of a GPCR is extracellular, is of variable length, and is often glycosylated. The carboxy-terminus is cytoplasmic and generally phosphorylated. Extracellular loops alternate with intracellular loops and link the transmembrane domains. Cysteine disulfide bridges linking the second and third extracellular loops may interact with agonists and antagonists. The most conserved domains of GPCRs are the transmembrane domains and the first two cytoplasmic loops. The transmembrane domains account, in part, for structural and functional features of the receptor. In most cases, the bundle of α helices forms a ligand-binding pocket. The extracellular N-terminal segment, or one or more of the three extracellular loops, may also participate in ligand binding. Ligand binding activates the receptor by inducing a conformational change in intracellular portions of the receptor. In turn, the large, third intracellular loop of the activated receptor interacts with a heterotrimeric guanine nucleotide binding (G) protein complex which mediates further intracellular signaling activities, including the activation of second messengers such as cyclic AMP (cAMP), phospholipase C, and inositol triphosphate, and the interaction of the activated GPCR with ion channel proteins. (See, e.g., Watson, S. and S. Arkinstall (1994) The G-protein Linked Receptor Facts Book, Academic Press, San Diego Calif., pp. 2-6; Bolander, F. F. (1994) Molecular Endocrinology, Academic Press, San Diego Calif., pp. 162-176; Baldwin, J. M. (1994) Curr. Opin. Cell Biol. 6:180-190.)

GPCRs include receptors for sensory signal mediators (e.g., light and olfactory stimulatory molecules); adenosine, γ-aminobutyric acid (GABA), hepatocyte growth factor, melanocortins, neuropeptide Y, opioid peptides, opsins, somatostatin, tachykinins, vasoactive intestinal polypeptide family, and vasopressin; biogenic amines (e.g., dopamine, epinephrine and norepinephrine, histamine, glutamate (metabotropic effect), acetylcholine (muscarinic effect), and serotonin); chemokines; lipid mediators of inflammation (e.g., prostaglandins and prostanoids, platelet activating factor, and leukotrienes); and peptide hormones (e.g., bombesin, bradykinin, calcitonin, C5a anaphylatoxin, endothelin, follicle-stimulating hormone (FSH), gonadotropic-releasing hormone (GnRH), neurokinin, and thyrotropin-releasing hormone (TRH), and oxytocin). GPCRs which act as receptors for stimuli that have yet to be identified are known as orphan receptors.

The diversity of the GPCR family is further increased by alternative splicing. Many GPCR genes contain introns, and there are currently over 30 such receptors for which splice variants have been identified. The largest number of variations are at the protein C-terminus. N-terminal and cytoplasmic loop variants are also frequent, while variants in the extracellular loops or transmembrane domains are less common. Some receptors have more than one site at which variance can occur. The splicing variants appear to be functionally distinct, based upon observed differences in distribution, signaling, coupling, regulation, and ligand binding profiles (Kilpatrick, G. J. et al. (1999) Trends Pharmacol. Sci. 20:294-301).

GPCRs can be divided into three major subfamilies: the rhodopsin-like, secretin-like, and metabotropic glutamate receptor subfamilies. Members of these GPCR subfamilies share similar functions and the characteristic seven transmembrane structure, but have divergent amino acid sequences. The largest family consists of the rhodopsin-like GPCRs, which transmit diverse extracellular signals including hormones, neurotransmitters, and light. Rhodopsin is a photosensitive GPCR found in animal retinas. In vertebrates, rhodopsin molecules are embedded in membranous stacks found in photoreceptor (rod) cells. Each rhodopsin molecule responds to a photon of light by triggering a decrease in cGMP levels which leads to the closure of plasma membrane sodium channels. In this manner, a visual signal is converted to a neural impulse. Other rhodopsin-like GPCRs are directly involved in responding to neurotransmitters. These GPCRs include the receptors for adrenaline (adrenergic receptors), acetylcholine (muscarinic receptors), adenosine, galanin, and glutamate (N-methyl-D-aspartate/NMDA receptors). (Reviewed in Watson, S. and S. Arkinstall (1994) The G-Protein Linked Receptor Facts Book, Academic Press, San Diego Calif., pp. 7-9, 19-22, 32-35, 130-131, 214-216, 221-222; Habert-Ortoli, E. et al. (1994) Proc. Natl. Acad. Sci. USA 91:9780-9783.)

The galanin receptors mediate the activity of the neuroendocrine peptide galanin, which inhibits secretion of insulin, acetylcholine, serotonin and noradrenaline, and stimulates prolactin and growth hormone release. Galanin receptors are involved in feeding disorders, pain, depression, and Alzheimer's disease (Kask, K. et al. (1997) Life Sci. 60:1523-1533). Other nervous system rhodopsin-like GPCRs include a growing family of receptors for lysophosphatidic acid and other lysophospholipids, which appear to have roles in development and neuropathology (Chun, J. et al. (1999) Cell Biochem. Biophys. 30:213-242).

The largest subfamily of GPCRs, the olfactory receptors, are also members of the rhodopsin-like GPCR family. These receptors function by transducing odorant signals. Numerous distinct olfactory receptors are required to distinguish different odors. Each olfactory sensory neuron expresses only one type of olfactory receptor, and distinct spatial zones of neurons expressing distinct receptors are found in nasal passages. For example, the RA1c receptor which was isolated from a rat brain library, has been shown to be limited in expression to very distinct regions of the brain and a defined zone of the olfactory epithelium (Raming, K. et al. (1998) Receptors Channels 6:141-151). However, the expression of olfactory-like receptors is not confined to olfactory tissues. For example, three rat genes encoding olfactory-like receptors having typical GPCR characteristics showed expression patterns not only in taste and olfactory tissue, but also in male reproductive tissue (Thomas, M. B. et al. (1996) Gene 178:1-5).

Members of the secretin-like GPCR subfamily have as their ligands peptide hormones such as secretin, calcitonin, glucagon, growth hormone-releasing hormone, parathyroid hormone, and vasoactive intestinal peptide. For example, the secretin receptor responds to secretin, a peptide hormone that stimulates the secretion of enzymes and ions in the pancreas and small intestine (Watson, supra, pp. 278-283). Secretin receptors are about 450 amino acids in length and are found in the plasma membrane of gastrointestinal cells. Binding of secretin to its receptor stimulates the production of cAMP.

Examples of secretin-like GPCRs implicated in inflammation and the immune response include the EGF module-containing, mucin-like hormone receptor (Emr1) and CD97 receptor proteins. These GPCRs are members of the recently characterized EGF-TM7 receptors subfamily. These seven transmembrane hormone receptors exist as heterodimers in vivo and contain between three and seven potential calcium-binding EGF-like motifs. CD97 is predominantly expressed in leukocytes and is markedly upregulated on activated B and T cells (McKnight, A. J. and S. Gordon (1998) J. Leukoc. Biol. 63:271-280).

The third GPCR subfamily is the metabotropic glutamate receptor family. Glutamate is the major excitatory neurotransmitter in the central nervous system. The metabotropic glutamate receptors modulate the activity of intracellular effectors, and are involved in long-term potentiation (Watson, supra, p.130). The Ca²⁺-sensing receptor, which senses changes in the extracellular concentration of calcium ions, has a large extracellular domain including clusters of acidic amino acids which may be involved in calcium binding. The metabotropic glutamate receptor family also includes pheromone receptors, the GABA_{B} receptors, and the taste receptors.

Other subfamilies of GPCRs include two groups of chemoreceptor genes found in the nematodes Caenorhabditis elegans and Caenorhabditis briggsae, which are distantly related to the mammalian olfactory receptor genes. The yeast pheromone receptors STE2 and STE3, involved in the response to mating factors on the cell membrane, have their own seven-transmembrane signature, as do the cAMP receptors from the slime mold Dictyostelium discoideum, which are thought to regulate the aggregation of individual cells and control the expression of numerous developmentally-regulated genes.

GPCR mutations, which may cause loss of function or constitutive activation, have been associated with numerous human diseases (Coughlin, supra). For instance, retinitis pigmentosa may arise from mutations in the rhodopsin gene. Furthermore, somatic activating mutations in the thyrotropin receptor have been reported to cause hyperfunctioning thyroid adenomas, suggesting that certain GPCRs susceptible to constitutive activation may behave as protooncogenes (Parma, J. et al. (1993) Nature 365:649-651). GPCR receptors for the following ligands also contain mutations associated with human disease: luteinizing hormone (precocious puberty); vasopressin V₂ (X-linked nephrogenic diabetes); glucagon (diabetes and hypertension); calcium (hyperparathyroidism, hypocalcuria, hypercalcemia); parathyroid hormone (short limbed dwarfism); β₃-adrenoceptor (obesity, non-insulin-dependent diabetes mellitus); growth hormone releasing hormone (dwarfism); and adrenocorticotropin (glucocorticoid deficiency) (Wilson, S. et al. (1998) Br. J. Pharmocol. 125:1387-1392; Stadel, J. M. et al. (1997) Trends Pharmacol. Sci. 18:43-0437). GPCRs are also involved in depression, schizophrenia, sleeplessness, hypertension, anxiety, stress, renal failure, and several cardiovascular disorders (Horn, F. and G. Vriend (1998) J. Mol. Med. 76:464-468).

In addition, within the past 20 years several hundred new drugs have been recognized that are directed towards activating or inhibiting GPCRs. The therapeutic targets of these drugs span a wide range of diseases and disorders, including cardiovascular, gastrointestinal, and central nervous system disorders as well as cancer, osteoporosis and endometriosis (Wilson et al., supra; Stadel et al., supra). For example, the dopamine agonist L-dopa is used to treat Parkinson's disease, while a dopamine antagonist is used to treat schizophrenia and the early stages of Huntington's disease. Agonists and antagonists of adrenoceptors have been used for the treatment of asthma, high blood pressure, other cardiovascular disorders, and anxiety; muscarinic agonists are used in the treatment of glaucoma and tachycardia; serotonin 5HT1D antagonists are used against migraine; and histamine H1 antagonists are used against allergic and anaphylactic reactions, hay fever, itching, and motion sickness (Horn et al., supra).

Recent research suggests potential future therapeutic uses for GPCRs in the treatment of metabolic disorders including diabetes, obesity, and osteoporosis. For example, mutant V2 vasopressin receptors causing nephrogenic diabetes could be functionally rescued in vitro by co-expression of a C-terminal V2 receptor peptide spanning the region containing the mutations. This result suggests a possible novel strategy for disease treatment (Schbneberg, T. et al. (1996) EMBO J. 15:1283-1291). Mutations in melanocortin-4 receptor (MC4R) are implicated in human weight regulation and obesity. As with the vasopressin V2 receptor mutants, these MC4R mutants are defective in trafficking to the plasma membrane (Ho, G. and R. G. MacKenzie (1999) J. Biol. Chem. 274:35816-35822), and thus might be treated with a similar strategy. The type 1 receptor for parathyroid hormone (PTH) is a GPCR that mediates the PTH-dependent regulation of calcium homeostasis in the bloodstream. Study of PTH/receptor interactions may enable the development of novel PTH receptor ligands for the treatment of osteoporosis (Mannstadt, M. et al. (1999) Am. J. Physiol. 277:F665-F675).

The chemokine receptor group of GPCRs have potential therapeutic utility in inflammation and infectious disease. (For review, see Locati, M. and P. M. Murphy (1999) Annu. Rev. Med. 50:425-440.) Chemokines are small polypeptides that act as intracellular signals in the regulation of leukocyte trafficking, hematopoiesis, and angiogenesis. Targeted disruption of various chemokine receptors in mice indicates that these receptors play roles in pathologic inflammation and in autoimmune disorders such as multiple sclerosis. Chemokine receptors are also exploited by infectious agents, including herpesviruses and the human immunodeficiency virus (HIV-1) to facilitate infection. A truncated version of chemokine receptor CCR5, which acts as a coreceptor for infection of T-cells by HIV-1, results in resistance to AIDS, suggesting that CCR5 antagonists could be useful in preventing the development of AIDS.

Nuclear Receptors

Nuclear receptors bind small molecules such as hormones or second messengers, leading to increased receptor-binding affinity to specific chromosomal DNA elements. In addition the affinity for other nuclear proteins may also be altered. Such binding and protein-protein interactions may regulate and modulate gene expression. Examples of such receptors include the steroid hormone receptors family, the retinoic acid receptors family, and the thyroid hormone receptors family.

Ligand-Gated Receptor Ion Channels

Ligand-gated receptor ion channels fall into two categories. The first category, extracellular ligand-gated receptor ion channels (ELGs), rapidly transduce neurotransmitter-binding events into electrical signals, such as fast synaptic neurotransmission. ELG function is regulated by post-translational modification. The second category, intracellular ligand-gated receptor ion channels (ILGs), are activated by many intracellular second messengers and do not require post-translational modification(s) to effect a channel-opening response.

ELGs depolarize excitable cells to the threshold of action potential generation. In non-excitable cells, ELGs permit a limited calcium ion-influx during the presence of agonist. ELGs include channels directly gated by neurotransmitters such as acetylcholine, L-glutamate, glycine, ATP, serotonin, GABA, and histamine. ELG genes encode proteins having strong structural and functional similarities. ILGs are encoded by distinct and unrelated gene families and include receptors for cAMP, cGMP, calcium ions, ATP, and metabolites of arachidonic acid.

Ligand-gated channels open their pores when an extracellular or intracellular mediator binds to the channel. Neurotransmitter-gated channels are channels that open when a neurotransmitter binds to their extracellular domain. These channels exist in the postsynaptic membrane of nerve or muscle cells. There are two types of neurotransmitter-gated channels. Sodium channels open in response to excitatory neurotransmitters, such as acetylcholine, glutamate, and serotonin. This opening causes an influx of Na⁺ and produces the initial localized depolarization that activates the voltage-gated channels and starts the action potential. Chloride channels open in response to inhibitory neurotransmitters, such as γ-aminobutyric acid (GABA) and glycine, leading to hyperpolarization of the membrane and the subsequent generation of an action potential. Neurotransmitter-gated ion channels have four transmembrane domains and probably function as pentamers (Jentsch, supra). Amino acids in the second transmembrane domain appear to be important in determining channel permeation and selectivity (Sather, W. A. et al. (1994) Curr. Opin. Neurobiol. 4:313-323).

Ligand-gated channels can be regulated by intracellular second messengers. For example, calcium-activated K⁺ channels are gated by internal calcium ions. In nerve cells, an influx of calcium during depolarization opens K⁺ channels to modulate the magnitude of the action potential (Ishi et al., supra . The large conductance (BK) channel has been purified from brain and its subunit composition determined. The α subunit of the BK channel has seven rather than six transmembrane domains in contrast to voltage-gated K⁺ channels. The extra transmembrane domain is located at the subunit N-terminus. A 28-amino-acid stretch in the C-terminal region of the subunit (the “calcium bowl” region) contains many negatively charged residues and is thought to be the region responsible for calcium binding. The β subunit consists of two transmembrane domains connected by a glycosylated extracellular loop, with intracellular N- and C-termini (Kaczorowski, supra; Vergara, C. et al. (1998) Curr. Opin. Neurobiol. 8:321-329).

Macrophage Scavenger Receptors

Macrophage scavenger receptors with broad ligand specificity may participate in the binding of low density lipoproteins (LDL) and foreign antigens. Scavenger receptors types I and II are trimeric membrane proteins with each subunit containing a small N-terminal intracellular domain, a transmembrane domain, a large extracellular domain, and a C-terminal cysteine-rich domain. The extracellular domain contains a short spacer domain, an α-helical coiled-coil domain, and a triple helical collagenous domain. These receptors have been shown to bind a spectrum of ligands, including chemically modified lipoproteins and albumin, polyribonucleotides, polysaccharides, phospholipids, and asbestos (Matsumoto, A. et al. (1990) Proc. Natl. Acad. Sci. USA 87:9133-9137; Elomaa, O. et al. (1995) Cell 80:603-609). The scavenger receptors are thought to play a key role in atherogenesis by mediating uptake of modified LDL in arterial walls, and in host defense by binding bacterial endotoxins, bacteria, and protozoa.

T-Cell Receptors

T cells play a dual role in the immune system as effectors and regulators, coupling antigen recognition with the transmission of signals that induce cell death in infected cells and stimulate proliferation of other immune cells. Although a population of T cells can recognize a wide range of different antigens, an individual T cell can only recognize a single antigen and only when it is presented to the T cell receptor (TCR) as a peptide complexed with a major histocompatibility molecule (MHC) on the surface of an antigen presenting cell. The TCR on most T cells consists of immunoglobulin-like integral membrane glycoproteins containing two polypeptide subunits, α and β, of similar molecular weight. Both TCR subunits have an extracellular domain containing both variable and constant regions, a transmembrane domain that traverses the membrane once, and a short intracellular domain (Saito, H. et al. (1984) Nature 309:757-762). The genes for the TCR subunits are constructed through somatic rearrangement of different gene segments. Interaction of antigen in the proper MHC context with the TCR initiates signaling cascades that induce the proliferation, maturation, and function of cellular components of the immune system (Weiss, A. (1991) Annu. Rev. Genet. 25:487-510). Rearrangements in TCR genes and alterations in TCR expression have been noted in lymphomas, leukemias, autoimmune disorders, and immunodeficiency disorders (Aisenberg, A. C. et al. (1985) N. Engl. J. Med. 313:529-533; Weiss, supra).

Netrin Receptors

The netrins are a family of molecules that function as diffusible attractants and repellants to guide migrating cells and axons to their targets within the developing nervous system. The netrin receptors include the C. elegans protein UNC-5, as well as homologues recently identified in vertebrates (Leonardo, E. D. et al. (1997) Nature 386:833-838). These receptors are members of the immunoglobulin superfamily, and also contain a characteristic domain called the ZU5 domain. Mutations in the mouse member of the netrin receptor family, Rcm (rostral cerebellar malformation) result in cerebellar and midbrain defects as an apparent result of abnormal neuronal migration (Ackerman, S. L. et al. (1997) Nature 386:838-842).

Interleukin Receptors

Interleukins (IL) mediate the interactions between immune and inflammatory cells. Several interleukins have been described; each has unique biological activities as well as some that overlap with the others. Macrophages produce IL-1 and IL-6, whereas T cells produce IL-2, IL-3, L-4, IL-5 and IL-6 and bone marrow stromal cells produce IL 7. IL 1 and IL 6 not only play important roles in immune cell function, but also stimulate a spectrum of inflammatory cell types. The growth and differentiation of eosinophils is markedly enhanced by IL 5. IL 2 is a potent proliferative signal for T cells, natural killer cells, and lymphokine-activated killer cells. IL 1, IL 3, IL 4, and IL 7 enhance the development of a variety of hematopoietic precursors. IL 4-IL 6 also serve to enhance B cell proliferation and antibody production (Mizel, S. B. (1989) FASEB J. 3:2379-2388).

Melatonin Receptors

Melatonin scavenges free radicals including the hydroxyl radical (—OH), peroxynitrite anion (ONOO—), and hypochlorous acid (HOCl), as well as preventing the translocation of nuclear factor-kappa B (NF-kappa B) to the nucleus and its binding to DNA, thereby reducing the upregulation of proinflammatory cytokines such as interleukins and tumor neurosis factor-alpha. Melatonin attenuates transendothelial cell migration and edema, which contribute to tissue damage (Reiter, R. J. et al. (2000) Ann. N.Y. Acad. Sci. 917:376-386). Activation of melatonin receptors enhances the release of T-helper cell cytokines, such as gamma-interferon and interleukin-2 (IL-2), as well as activation of opioid cytokines which crossreact immunologically with both interleukin-4 and dynorphin B. Hematopoiesis is influenced by melatonin-induced-opioids acting on kappa 1-opioid receptors present on bone marrow macrophages (Maestroni, G. J. (1999) Adv. Exp. Med. Biol. 467:217-226).

VPS10 Domain Containing Receptors

The members of the VPS10 domain containing receptor family all contain a domain with homology to the yeast vacuolar sorting protein 10 (VPS10) receptor. This family includes the mosaic receptor SorLA, the neurotensin receptor sordlin, and SorCS, which is expressed during mouse embryonal and early postnatal nervous system development (Hermey, G. et al. (1999) Biochem. Biophys. Res. Commun. 266:347-351; Hermey, G. et al. (2001) Neuroreport 12:29-32).

Neurotensin is a brain and gastrointestinal peptide that fulfils many functions through its interaction with specific receptors. Subtypes of neurotensin receptors include two G protein-coupled receptors, and the neuropeptide receptor sortilin, a 100 kDa-protein with a single transmembrane domain (Vincent, J. P. et al. (1999) Trends Pharmacol Sci 20:302-309). Sortilin, a multiligand type-1 receptor with homology to the yeast receptor Vps10p, is a sorting receptor for ligands in the synthetic pathway as well as on the cell membrane. Sortilin is a mammalian receptor targeted by the GGA family of cytosolic sorting proteins, which condition the Vps10p-mediated sorting of yeast carboxypeptidase Y (Nielsen, M. S. et al. (2001) EMBO J. 20:2180-2190). SorCS, SorLA and the neurotensin receptor sortilin share a common VPS10 domain. In the N-terminus of SorCS two putative cleavage sites for the convertase furin mark the beginning of the VPS10 domain, followed by a module of imperfect leucine-rich repeats and a transmembrane domain. The short intracellular C-terminus contains consensus signals for rapid internalization. SorCS is predominantly expressed in brain, but also in heart, liver, and kidney (Hermey G. et al. (1999) Biochem. Biophys. Res Commun. 266:347-351). SorCS2 is highly expressed in the developing and mature mouse central nervous system. Its main site of expression is the floor plate, and high levels are also detected transiently in brain regions including the dopaminergic brain nuclei and the dorsal thalamus (Rezgaoui, M. (2001) Mech. Dev. 100:335-338).

Munc13 Proteins

Munc13 proteins constitute a family of molecules (Munc13-1, Munc13-2, Munc 13-3, and Munc 13-4) with homology to Caenorhabditis elegans unc-13p. Munc13 proteins contain a phorbol ester-binding C1-domain and two C2-domains, which are Ca²⁺/phospholipid binding domains. With the exception of a ubiquitously expressed Munc13-2 splice variant and a predominantly lung-specific Munc 13-4 isoform, Munc13 proteins are specifically expressed in the brain, where in excitatory/glutamatergic neurons, M13 proteins play a central role in neurotransmitter-specific synaptic vesicle priming. For example, Munc13-1, which is targeted to presynaptic active zones, binds to syntaxin, a component of the synaptic vesicle fusion apparatus and acts as a phorbol ester-dependent enhancer of neurotransmitter secretion. Loss of Munc13-1 in deletion mutant mice leads to an arrest of the synaptic vesicle cycle of hippocampal neurons at the synaptic vesicle priming step, resulting in a functional shutdown of synapses (Augustin, I. et al. (1999) Nature 400:457-461; Koch, H. et al. (2000) Biochem. J. 349:247-253). Recently, Munc13-3, which is specifically expressed in the cerebellum, is proposed to act at a similar step of the synaptic vesicle cycle as does Munc13-1 (Augustin, I. et al. (2001) J. Neurosci 21:10-17).

### Membrane-Associated Proteins

Tetraspan Family Proteins

The transmembrane 4 superfamily (TM4SF) or tetraspan family is a multigene family encoding type III integral membrane proteins (Wright, M. D. and M. G. Tomlinson (1994) Immunol. Today 15:588-594). The TM4SF is comprised of membrane proteins which traverse the cell membrane four times. Members of the TM4SF include platelet and endothelial cell membrane proteins, melanoma-associated antigens, leukocyte surface glycoproteins, colonal carcinoma antigens, tumor-associated antigens, and surface proteins of the schistosome parasites (Jankowski, S. A. (1994) Oncogene 9:1205-1211). Members of the TM4SF share about 25-30% amino acid sequence identity with one another. A number of TM4SF members have been implicated in signal transduction, control of cell adhesion, regulation of cell growth and proliferation, including development and oncogenesis, and cell motility, including tumor cell metastasis. Expression of TM4SF proteins,is associated with a variety of tumors and the level of expression may be altered when cells are growing or activated.

Tumor Antigens

Tumor antigens are surface molecules that are differentially expressed in tumor cells relative to normal cells. Tumor antigens distinguish tumor cells immunologically from normal cells and provide diagnostic and therapeutic targets for human cancers (Takagi, S. et al. (1995) Int. J. Cancer 61:706-715; Liu, E. et al. (1992) Oncogene 7:1027-1032).

Ion Channels

Ion channels are found in the plasma membranes of virtually every cell in the body. For example, chloride channels mediate a variety of cellular functions including regulation of membrane potentials and absorption and secretion of ions across epithelial membranes. When present in intracellular membranes of the Golgi apparatus and endocytic vesicles, chloride channels also regulate organelle pH. (See, e.g., Greger, R. (1988) Annu. Rev. Physiol. 50:111-122.) Electrophysiological and pharmacological properties of chloride channels, including ion conductance, current-voltage relationships, and sensitivity to modulators, suggest that different chloride channels exist in muscles, neurons, fibroblasts, epithelial cells, and lymphocytes. Many channels have sites for phosphorylation by one or more protein kinases including protein kinase A, protein kinase C, tyrosine kinase, and casein kinase II, all of which regulate ion channel activity in cells. Inappropriate phosphorylation of proteins in cells has been linked to changes in cell cycle progression and cell differentiation. Changes in the cell cycle have been linked to induction of apoptosis or cancer. Changes in cell differentiation have been linked to diseases and disorders of the reproductive system, immune system, and skeletal muscle.

The electrical potential of a cell is generated and maintained by controlling the movement of ions across the plasma membrane. The movement of ions requires ion channels, which form ion-selective pores within the membrane. There are two basic types of ion channels, ion transporters and gated ion channels. Ion transporters utilize the energy obtained from ATP hydrolysis to actively transport an ion against the ion's concentration gradient. Gated ion channels allow passive flow of an ion down the ion's electrochemical gradient under restricted conditions. Together, these types of ion channels generate, maintain, and utilize an electrochemical gradient that is used in 1) electrical impulse conduction down the axon of a nerve cell, 2) transport of molecules into cells against concentration gradients, 3) initiation of muscle contraction, and 4) endocrine cell secretion.

Ion transporters generate and maintain the resting electrical potential of a cell. Utilizing the energy derived from ATP hydrolysis, they transport ions against the ion's concentration gradient. These transmembrane ATPases are divided into three families. The phosphorylated (P) class ion transporters, including Na⁺-K⁺ ATPase, Ca²⁺-ATPase, and H⁺-ATPase, are activated by a phosphorylation event. P-class ion transporters are responsible for maintaining resting potential distributions such that cytosolic concentrations of Na⁺ and Ca²⁺ are low and cytosolic concentration of K⁺ is high. The vacuolar (V) class of ion transporters includes H⁺ pumps on intracellular organelles, such as lysosomes and Golgi. V-class ion transporters are responsible for generating the low pH within the lumen of these organelles that is required for function. The coupling factor (F) class consists of H⁺ pumps in the mitochondria. F-class ion transporters utilize a proton gradient to generate ATP from ADP and inorganic phosphate (P₁).

The P-ATPases are hexamers of a 100 kD subunit with ten transmembrane domains and several large cytoplasmic regions that may play a role in ion binding (Scarborough, G. A. (1999) Curr. Opin. Cell Biol. 11:517-522). The V-ATPases are composed of two functional domains: the V₁ domain, a peripheral complex responsible for ATP hydrolysis; and the V₀ domain, an integral complex responsible for proton translocation across the membrane. The F-ATPases are structurally and evolutionarily related to the V-ATPases. The P-ATPase F₀ domain contains 12 copies of the c subunit, a highly hydrophobic protein composed of two transmembrane domains and containing a single buried carboxyl group in TM2 that is essential for proton transport. The V-ATPase V₀ domain contains three types of homologous c subunits with four or five transmembrane domains and the essential carboxyl group in TM4 or TM3. Both types of complex also contain a single a subunit that may be involved in regulating the pH dependence of activity (Forgac, M. (1999) J. Biol. Chem. 274:12951-12954).

The resting potential of the cell is utilized in many processes involving carrier proteins and gated ion channels. Carrier proteins utilize the resting potential to transport molecules into and out of the cell. Amino acid and glucose transport into many cells is linked to sodium ion co-transport (symport) so that the movement of Na⁺ down an electrochemical gradient drives transport of the other molecule up a concentration gradient. Similarly, cardiac muscle links transfer of Ca²⁺ out of the cell with transport of Na⁺ into the cell (antiport).

Gated ion channels control ion flow by regulating the opening and closing of pores. The ability to control ion flux through various gating mechanisms allows ion channels to mediate such diverse signaling and homeostatic functions as neuronal and endocrine signaling, muscle contraction, fertilization, and regulation of ion and pH balance. Gated ion channels are categorized according to the manner of regulating the gating function. Mechanically-gated channels open their pores in response to mechanical stress; voltage-gated channels (e.g., Na⁺, K⁺, Ca²⁺, and Cl⁻ channels) open their pores in response to changes in membrane potential; and ligand-gated channels (e.g., acetylcholine-, serotonin-, and glutamate-gated cation channels, and GABA- and glycine-gated chloride channels) open their pores in the presence of a specific ion, nucleotide, or neurotransmitter. The gating properties of a particular ion channel (i.e., its threshold for and duration of opening and closing) are sometimes modulated by association with auxiliary channel proteins and/or post translational modifications, such as phosphorylation.

Mechanically-gated or mechanosensitive ion channels act as transducers for the senses of touch, hearing, and balance, and also play important roles in cell volume regulation, smooth muscle contraction, and cardiac rhythm generation. A stretch-inactivated channel (SIC) was recently cloned from rat kidney. The SIC channel belongs to a group of channels which are activated by pressure or stress on the cell membrane and conduct both Ca²⁺ and Na⁺ (Suzuki, M. et al. (1999) J. Biol. Chem. 274:6330-6335).

The pore-forming subunits of the voltage-gated cation channels form a superfamily of ion channel proteins. The characteristic domain of these channel proteins comprises six transmembrane domains (S1-S6), a pore-forming region (P) located between S5 and S6, and intracellular amino and carboxy termini. In the Na⁺ and Ca²⁺ subfamilies, this domain is repeated four times, while in the K⁺ channel subfamily, each channel is formed from a tetramer of either identical or dissimilar subunits. The P region contains information specifying the ion selectivity for the channel. In the case of K⁺ channels, a GYG tripeptide is involved in this selectivity (Ishii, T. M. et al. (1997) Proc. Natl. Acad. Sci. USA 94:11651-11656).

Voltage-gated Na⁺ and K⁺ channels are necessary for the function of electrically excitable cells, such as nerve and muscle cells. Action potentials, which lead to neurotransmitter release and muscle contraction, arise from large, transient changes in the permeability of the membrane to Na⁺ and K⁺ ions. Depolarization of the membrane beyond the threshold level opens voltage-gated Na⁺ channels. Sodium ions flow into the cell, further depolarizing the membrane and opening more voltage-gated Na⁺ channels, which propagates the depolarization down the length of the cell. Depolarization also opens voltage-gated potassium channels. Consequently, potassium ions flow outward, which leads to repolarization of the membrane. Voltage-gated channels utilize charged residues in the fourth transmembrane segment (S4) to sense voltage change. The open state lasts only about 1 millisecond, at which time the channel spontaneously converts into an inactive state that cannot be opened irrespective of the membrane potential. Inactivation is mediated by the channel's N-terminus, which acts as a plug that closes the pore. The transition from an inactive to a closed state requires a return to resting potential.

Voltage-gated Na⁺ channels are heterotrimeric complexes composed of a 260 kDa pore-forming a subunit that associates with two smaller auxiliary subunits, β1 and β2. The β2 subunit is a integral membrane glycoprotein that contains an extracellular Ig domain, and its association with α and β1 subunits correlates with increased functional expression of the channel, a change in its gating properties, as well as an increase in whole cell capacitance due to an increase in membrane surface area (Isom, L. L. et al. (1995) Cell 83:433-442).

Non voltage-gated Na⁺ channels include the members of the amiloride-sensitive Na⁺ channel/degenerin (NaC/DEG) family. Channel subunits of this family are thought to consist of two transmembrane domains flanking a long extracellular loop, with the amino and carboxyl termini located within the cell. The NaC/DEG family includes the epithelial Na⁺ channel (ENaC) involved in Na⁺ reabsorption in epithelia including the airway, distal colon, cortical collecting duct of the kidney, and exocrine duct glands. Mutations in ENaC result in pseudohypoaldosteronism type 1 and Liddle's syndrome (pseudohyperaldosteronism). The NaC/DEG family also includes the recently characterized H⁺-gated cation channels or acid-sensing ion channels (ASIC). ASIC subunits are expressed in the brain and form heteromultimeric Na⁺-permeable channels. These channels require acid pH fluctuations for activation. ASIC subunits show homology to the degenerins, a family of mechanically-gated channels originally isolated from C. elegans. Mutations in the degenerins cause neurodegeneration. ASIC subunits may also have a role in neuronal function, or in pain perception, since tissue acidosis causes pain (Waldmann, R. and M. Lazdunski (1998) Curr. Opin. Neurobiol. 8:418-424; Eglen, R. M. et al. (1999) Trends Pharmacol. Sci. 20:337-342).

K⁺ channels are located in all cell types, and may be regulated by voltage, ATP concentration, or second messengers such as Ca²⁺ and cAMP. In non-excitable tissue, K⁺ channels are involved in protein synthesis, control of endocrine secretions, and the maintenance of osmotic equilibrium across membranes. In neurons and other excitable cells, in addition to regulating action potentials and repolarizing membranes, K⁺ channels are responsible for setting resting membrane potential. The cytosol contains non-diff-usible anions and, to balance this net negative charge, the cell contains a Na⁺-K⁺ pump and ion channels that provide the redistribution of Na⁺, K⁺, and Cl⁻. The pump actively transports Na⁺ out of the cell and K⁺ into the cell in a 3:2 ratio. Ion channels in the plasma membrane allow K⁺ and Cl⁻ to flow by passive diffusion. Because of the high negative charge within the cytosol, Cl⁻ flows out of the cell. The flow of K⁺ is balanced by an electromotive force pulling K⁺ into the cell, and a K⁺ concentration gradient pushing K⁺ out of the cell. Thus, the resting membrane potential is primarily regulated by K⁺ flow (Salkoff, L. and T. Jegla (1995) Neuron 15:489-492).

The voltage-gated Ca²⁺ channels have been classified into several subtypes based upon their electrophysiological and pharmacological characteristics. L-type Ca²⁺ channels are predominantly expressed in heart and skeletal muscle where they play an essential role in excitation-contraction coupling. T-type channels are important for cardiac pacemaker activity, while N-type and P/Q-type channels are involved in the control of neurotransmitter release in the central and peripheral nervous system. The L-type and N-type voltage-gated Ca²⁺ channels have been purified and, though their functions differ dramatically, they have similar subunit compositions. The channels are composed of three subunits. The α₁ subunit forms the membrane pore and voltage sensor, while the α₂δ and β subunits modulate the voltage-dependence, gating properties, and the current amplitude of the channel. These subunits are encoded by at least six α₁, one α₂δ, and four β genes. A fourth subunit, γ, has been identified in skeletal muscle (Walker, D. et al. (1998) J. Biol. Chem. 273:2361-2367; McCleskey, E. W. (1994) Curr. Opin. Neurobiol. 4:304-312).

The transient receptor family (Trp) of calcium ion channels are thought to mediate capacitative calcium entry (CCE). CCE is the Ca²⁺ influx into cells to resupply Ca²⁺ stores depleted by the action of inositol triphosphate (IP3) and other agents in response to numerous hormones and growth factors. Trp and Trp-like were first cloned from Drosophila and have similarity to voltage gated Ca²⁺ channels in the S3 through S6 regions. This suggests that Trp and/or related proteins may form mammalian CCC entry channels (Zhu, X. et al. (1996) Cell 85:661-671; Boulay, G. et al. (1997) J. Biol. Chem. 272:29672-29680). Melastatin is a gene isolated in both the mouse and human, and whose expression in melanoma cells is inversely correlated with melanoma aggressiveness in vivo. The human cDNA transcript corresponds to a 1533-amino acid protein having homology to members of the Trp family. It has been proposed that the combined use of malastatin mRNA expression status and tumor thickness might allow for the determination of subgroups of patients at both low and high risk for developing metastatic disease (Duncan, L. M. et al (2001) J. Clin. Oncol. 19:568-576).

Chloride channels are necessary in endocrine secretion and in regulation of cytosolic and organelle pH. In secretory epithelial cells, Cl⁻ enters the cell across a basolateral membrane through an Na⁺, K⁺ /Cl⁻ cotransporter, accumulating in the cell above its electrochemical equilibrium concentration. Secretion of Cl⁻ from the apical surface, in response to hormonal stimulation, leads to flow of Na⁺ and water into the secretory lumen. The cystic fibrosis transmembrane conductance regulator (CFTR) is a chloride channel encoded by the gene for cystic fibrosis, a common fatal genetic disorder in humans. CFTR is a member of the ABC transporter family, and is composed of two domains each consisting of six transmembrane domains followed by a nucleotide-binding site. Loss of CFTR function decreases transepithelial water secretion and, as a result, the layers of mucus that coat the respiratory tree, pancreatic ducts, and intestine are dehydrated and difficult to clear. The resulting blockage of these sites leads to pancreatic insufficiency, “meconium ileus”, and devastating “chronic obstructive pulmonary disease” (A1-Awqati, Q. et al. (1992) J. Exp. Biol. 172:245-266).

The voltage-gated chloride channels (CLC) are characterized by 10-12 transmembrane domains, as well as two small globular domains known as CBS domains. The CLC subunits probably function as homotetramers. CLC proteins are involved in regulation of cell volume, membrane potential stabilization, signal transduction, and transepithelial transport. Mutations in CLC-1, expressed predominantly in skeletal muscle, are responsible for autosomal recessive generalized myotonia and autosomal dominant myotonia congenita, while mutations in the kidney channel CLC-5 lead to kidney stones (Jentsch, T. J. (1996) Curr. Opin. Neurobiol. 6:303-310).

Cyclic nucleotide-gated (CNG) channels are gated by cytosolic cyclic nucleotides. The best examples of these are the cAMP-gated Na⁺ channels involved in olfaction and the cGMP-gated cation channels involved in vision. Both systems involve ligand-mediated activation of a G-protein coupled receptor which then alters the level of cyclic nucleotide within the cell. CNG channels also represent a major pathway for Ca²⁺ entry into neurons, and play roles in neuronal development and plasticity. CNG channels are tetramers containing at least two types of subunits, an α subunit which can form functional homomeric channels, and β subunit, which modulates the channel properties. All CNG subunits have six transmembrane domains and a pore forming region between the fifth and sixth transmembrane domains, similar to voltage-gated K⁺ channels. A large C-terminal domain contains a cyclic nucleotide binding domain, while the N-terminal domain confers variation among channel subtypes (Zufall, F. et al. (1997) Curr. Opin. Neurobiol. 7:404-412).

The activity of other types of ion channel proteins may also be modulated by a variety of intracellular signalling proteins. Many channels have sites for phosphorylation by one or more protein kinases including protein kinase A, profein kinase C, tyrosine kinase, and casein kinase II, all of which regulate ion channel activity in cells. Kir channels are activated by the binding of the Gβγ subunits of heterotrimeric G-proteins (Reimann, F. and F. M. Ashcroft (1999) Curr. Opin. Cell. Biol. 11:503-508). Other proteins are involved in the localization of ion channels to specific sites in the cell membrane. Such proteins include the PDZ domain proteins known as MAGUKs (membrane-associated guanylate kinases) which regulate the clustering of ion channels at neuronal synapses (Craven, S. E. and D. S. Bredt (1998) Cell 93:495-498). Cerebellar granule neurons possess a non-inactivating potassium current which modulates firing frequency upon receptor stimulation by neurotransmitters and controls the resting membrane potential. Potassium channels that exhibit non-inactivating currents include the ether a go-go (EAG) channel. A membrane protein designated KCR1 specifically binds to rat EAG by means of its C-terminal region and regulates the cerebellar non-inactivating potassium current. KCR1 is predicted to contain 12 transmembrane domains, with intracellular amino and carboxyl termini. Structural characteristics of these transmembrane regions appear to be similar to those of the transporter superfamily, but no homology between KCR1 and known transporters was found, suggesting that KCR1 belongs to a novel class of transporters. KCR1 appears to be the regulatory component of non-inactivating potassium channels (Hoshi, N. et al. (1998) J. Biol. Chem. 273:23080-23085).

Proton ATPases are a large class of membrane proteins that use the energy of ATP hydrolysis to generate an electrochemical proton gradient across a membrane. The resultant gradient may be used to transport other ions across the membrane (Na⁺, K⁺, or Cl⁻) or to maintain organelle pH. Proton ATPases are further subdivided into the mitochondrial F-ATPases, the plasma membrane ATPases, and the vacuolar ATPases. The vacuolar ATPases establish and maintain an acidic pH within various vesicles involved in the processes of endocytosis and exocytosis (Mellman, I. et al. (1986) Ann. Rev. Biochem. 55:663-700).

Proton-coupled, 12 membrane-spanning domain transporters such as PEPT 1 and PEPT 2 are responsible for gastrointestinal absorption and for renal reabsorption of peptides using an electrochemical H⁺ gradient as the driving force. Another type of peptide transporter, the TAP transporter, is a heterodimer consisting of TAP 1 and TAP 2 and is associated with antigen processing. Peptide antigens are transported across the membrane of the endoplasmic reticulum by TAP so they can be expressed on the cell surface in association with MHC molecules. Each TAP protein consists of multiple hydrophobic membrane spanning segments and a highly conserved ATP-binding cassette (Boll, M. et al. (1996) Proc. Natl. Acad. Sci. 93:284-289). Pathogenic microorganisms, such as herpes simplex virus, may encode inhibitors of TAP-mediated peptide transport in order to evade immune surveillance (Marusina, K. and Manaco, J. J. (1996) Curr. Opin. Hematol. 3:19-26).

Semaphorins and Neuropilins

Semaphorins are a large group of axonal guidance molecules consisting of at least 30 different members and are found in vertebrates, invertebrates, and even certain viruses. Semaphorins comprise a family of both secreted and transmembrane glycoproteins and have a well-conserved extracellular domain of about 500 amino acids. As the name of the family implies, the function of semaphorins is growth cone guidance. At least two secreted seniaphorins, Sema II and Sema III, function by repelling (i.e., by causing the collapse of) growth cones. Sema III causes the collapse of neuronal growth cones. Neuropilin was originally identified as an axonal glycoprotein. More recent evidence suggests that neuropilin is a high-affinity semaphorin receptor specific for SemaIII. The extracellular region of neuropilins consists of three different domains: CUB, discoidin, and MAM domains. The CUB and the MAM motifs of neuropilin have been suggested to have roles in protein-protein interactions and are thought to be involved in the binding of semaphorins through the sema and the C-terminal domains (reviewed in Raper, J. A. (2000) Curr. Opin. Neurobiol. 10:88-94). Binding appears to involve a CUB (complement binding) domain, coagulation factor domain, and MAM domain (also found in metalloendopeptidases, receptor protein kinases, and macrophage-specific scavenger receptors) (Kolodkin, A. L, et al. (1997) Cell 90:753-762; and references within).

Membrane Proteins Associated with Intercellular Communication

Intercellular communication is essential for the development and survival of multicellular organisms. Cells communicate with one another through the secretion and uptake of protein signaling molecules. The uptake of proteins into the cell is achieved by endocytosis, in which the interaction of signaling molecules with the plasma membrane surface, often via binding to specific receptors, results in the formation of plasma membrane-derived vesicles that enclose and transport the molecules into the cytosol. The secretion of proteins from the cell is achieved by exocytosis, in which molecules inside of the cell are packaged into membrane-bound transport vesicles derived from the trans Golgi network. These vesicles fuse with the plasma membrane and release their contents into the surrounding extracellular space. Endocytosis and exocytosis result in the removal and addition of plasma membrane components, and the recycling of these components is essential to maintain the integrity, identity, and functionality of both the plasma membrane and internal membrane-bound compartments.

Nogo has been identified as a component of the central nervous system myelin that prevents axonal regeneration in adult vertebrates. Cleavage of the Nogo-66 receptor and other glycophosphatidylinositol-linked proteins from axonal surfaces renders neurons insensitive to Nogo-66, facilitating potential recovery from CNS damage (Fournier, A. E. et al. (2001) Nature 409:341-346).

The slit proteins are extracellular matrix proteins expressed by cells at the ventral midline of the nervous system. Slit proteins are ligands for the repulsive guidance receptor Roundabout (Robo) and thus play a role in repulsive axon guidance (Brose, K. et al. (1999) Cell 96:795-806).

Lysosomes are the site of degradation of intracellular material during autophagy and of extracellular molecules following endocytosis. Lysosomal enzymes are packaged into vesicles which bud from the trans-Golgi network. These vesicles fuse with endosomes to form the mature lysosome in which hydrolytic digestion of endocytosed material occurs. Lysosomes can fuse with autophagosomes to form a unique compartment in which the degradation of organelles and other intracellular components occurs.

Protein sorting by transport vesicles, such as the endosome, has important consequences for a variety of physiological processes including cell surface growth, the biogenesis of distinct intracellular organelles, endocytosis, and the controlled secretion of hormones and neurotransmitters (Rothman, J. E. and F. T. Wieland (1996) Science 272:227-234). In particular, neurodegenerative disorders and other neuronal pathologies are associated with biochemical flaws during endosomal protein sorting or endosomal biogenesis (Mayer, R. J. et al. (1996) Adv. Exp. Med. Biol. 389:261-269).

Peroxisomes are organelles independent from the secretory pathway. They are the site of many peroxide-generating oxidative reactions in the cell. Peroxisomes are unique among eukaryotic organelles in that their size, number, and enzyme content vary depending upon organism, cell type, and metabolic needs (Waterham, H. R. and J. M. Cregg (1997) BioEssays 19:57-66). Genetic defects in peroxisome proteins which result in peroxisomal deficiencies have been linked to a number of human pathologies, including Zellweger syndrome, rhizomelic chonrodysplasia punctata, X-linked adrenoleukodystrophy, acyl-CoA oxidase deficiency, bifunctional enzyme deficiency, classical Refsum's disease, DHAP alkyl transferase deficiency, and acatalasemia (Moser, H. W. and A. B. Moser (1996) Ann. NY Acad. Sci. 804:427-441). In addition, Gartner, J. et al. (1991; Pediatr. Res. 29:141-146) found a 22 kDa integral membrane protein associated with lower density peroxisome-like subcellular fractions in patients with Zellweger syndrome.

Polycystin-1 is the protein product of the polycystic kidney disease-1 (PKD1) gene. Mutations in PKD1 and PKD2 are responsible for almost all cases of autosomal dominant polycystic kidney disease (Sandford, R. et al. (1999) Cell Mol. Life Sci. 56:567-579). Polycystin-1 functions as a matrix receptor to link the extracellular matrix to the actin cytoskeleton via focal adhesion proteins. Polycystin-1 is highly expressed in the basal membranes of ureteric bud epithelia during early development of the metanephric kidney. Polycystin-1 forms multiprotein complexes with alpha2beta1-integrin, talin, vinculin, paxillin, p130cas, focal adhesion kinase, and c-src in normal human fetal collecting tubules. In normal adult kidneys, polycystin-1 is downregulated and forms complexes with the cell-cell adherens junction proteins E-cadherin and beta-, gamma-, and alpha-catenin (Wilson, P. D. (2001) J. Am. Soc. Nephrol.12:834-45).

Normal embryonic development and control of germ cell maturation is modulated by a number of secretory proteins which interact with their respective membrane-bound receptors. Cell fate during embryonic development is determined by members of the activin/TGF-β superfamily, cadherins, IGF-2, and other morphogens. In addition, proliferation, maturation, and redifferentiation of germ cell and reproductive tissues are regulated, for example, by IGF-2, inhibins, activins, and follistatins (Petraglia, F. (1997) Placenta 18:3-8; Mather, J. P. et al. (1997) Proc. Soc. Exp. Biol. Med. 215:209-222). Transforming growth factor beta (TGFbeta) signal transduction is mediated by two receptor Ser/Thr kinases acting in series, type II TGFbeta receptor and (TbetaR-II) phosphorylating type I TGFbeta receptor (ThetaR-I). TbetaR-I-associated protein-1 (TRECAP-1), which distinguishes between quiescent and activated forms of the type I transforming growth factor beta receptor, has been associated with TGFbeta signaling (Charng, M. J. et al. (1998) J. Biol. Chem. 273:9365-9368).

Retinoic acid receptor alpha (RAR alpha) mediates retinoic-acid induced maturation and has been implicated in myeloid development. Genes induced by retinoic acid during granulocytic differentiation include E3, a hematopoietic-specific gene that is an immnediate target for the activated RAR alpha during myelopoiesis (Scott, L. M. et al. (1996) Blood 88:2517-2530).

The μ-opioid receptor (MOR) mediates the actions of analgesic agents including morphine, codeine, methadone, and fentanyl as well as heroin. MOR is functionally coupled to a G-protein-activated potassium channel (Mestek A. et al. (1995) J. Neurosci. 15:2396-2406). A variety of MOR subtypes exist. Alternative splicing has been observed with MOR-1 as with a number of G protein-coupled receptors including somatostatin 2, dopamine D2, prostaglandin EP3, and serotonin receptor subtypes 5-hydroxytryptamine4 and 5-hydroxytryptamine7 (Pan, Y. X. et al. (1999) Mol. Pharm. 56:396-403).

Peripheral and Anchored Membrane Proteins

Some membrane proteins are not membrane-spanning but are attached to the plasma membrane via membrane anchors or interactions with integral membrane proteins. Membrane anchors are covalently joined to a protein post-translationally and include such moieties as prenyl, myristyl, and glycosylphosphatidyl inositol groups. Membrane localization of peripheral and anchored proteins is important for their function in processes such as receptor-mediated signal transduction. For example, prenylation of Ras is required for its localization to the plasma membrane and for its normal and oncogenic functions in signal transduction.

T Cell Activation

Human T cells can be specifically activated by Staphyloccocal exotoxins, resulting in cytokine production and cell proliferation which can lead to septic shock (Muraille, E. et al. (1999) Int. Immunol. 11:1403-1410). Activation of T cells by Staphyloccocal exotoxins requires the presence of antigen presenting cells (APC) to present the exotoxin molecules to the T cells and to deliver the costimulatory signals required for optimum T cell activation. Although Staphyloccocal exotoxins must be presented to T cells by APC, these molecules do not require processing by APC. Instead, Staphyloccocal exotoxins directly bind to a non-polymorphic portion of the human major histocompatibility complex (MHC) class II molecules, thus bypassing the need for capture, cleavage, and binding of the peptides to the polymorphic antigenic groove of the MHC class II molecules.

### Endoplasmic Reticulum Membrane Proteins

The normal functioning of the eukaryotic cell requires that all newly synthesized proteins be correctly folded, modified, and delivered to specific intra- and extracellular sites. Newly synthesized membrane and secretory proteins enter a cellular sorting and distribution network during or immediately after synthesis and are routed to specific locations inside and outside of the cell. The initial compartment in this process is the endoplasmic reticulum (ER) where proteins undergo modifications such as glycosylation, disulfide bond formation, and oligomerization. The modified proteins are then transported through a series of membrane-bound compartments which include the various cisternae of the Golgi complex, where further carbohydrate modifications occur. Transport between compartments occurs by means of vesicle budding and fusion. Once within the secretory pathway, proteins do not have to cross a membrane to reach the cell surface.

Although the majority of proteins processed through the ER are transported out of the organelle, some are retained. The signal for retention in the ER in mammalian cells consists of the tetrapeptide sequence, KDEL, located at the carboxyl terminus of resident ER membrane proteins (Munro, S. (1986) Cell 46:291-300). Proteins containing this sequence leave the ER but are quickly retrieved from the early Golgi cisternae and returned to the ER, while proteins lacking this signal continue through the secretory pathway.

Disruptions in the cellular secretory pathway have been implicated in several human diseases. In familial hypercholesterolemia the low density lipoprotein receptors remain in the ER, rather than moving to the cell surface (Pathak, R. K. (1988) J. Cell Biol. 106:1831-1841). Altered transport and processing of the β-amyloid precursor protein (PAPP) involves the putative vesicle transport protein presenilin and may play a role in early-onset Alzheimer's disease (Levy-Lahad, E. et al. (1995) Science 269:973-977). Changes in ER-derived calcium homeostasis have been associated with diseases such as cardiomyopathy, cardiac hypertrophy, myotonic dystrophy, Brody disease, Smith-McCort dysplasia, and diabetes mellitus.

### Mitochondrial Membrane Proteins

The mitochondrial electron transport (or respiratory) chain is a series of three enzyme complexes in the mitochondrial membrane that is responsible for the transport of electrons from NADH to oxygen and the coupling of this oxidation to the synthesis of ATP (oxidative phosphorylation). ATP then provides the primary source of energy for driving the many energy-requiring reactions of a cell.

Most of the protein components of the mitochondrial respiratory chain are the products of nuclear encoded genes that are imported into the mitochondria, and the remainder are products of mitochondrial genes. Defects and altered expression of enzymes in the respiratory chain are associated with a variety of disease conditions in man, including, for example, neurodegenerative diseases, myopathies, and cancer.

### Lymphocyte and Leukocyte Membrane Proteins

The B-cell response to antigens is an essential component of the normal immune system. Mature B cells recognize foreign antigens through B cell receptors (BCR) which are membrane-bound, specific antibodies that bind foreign antigens. The antigen/receptor complex is internalized, and the antigen is proteolytically processed. To generate an efficient response to complex antigens, the BCR, BCR-associated proteins, and T cell response are all required. Proteolytic fragments of the antigen are complexed with major histocompatability complex-II (MHCII) molecules on the surface of the B cells where the complex can be recognized by T cells. In contrast, macrophages and other lymphoid cells present antigens in association with MHCI molecules to T cells. T cells recognize and are activated by the MHCI-antigen complex through interactions with the T cell receptor/CD3 complex, a T cell-surface multimeric protein located in the plasma membrane. T cells activated by antigen presentation secrete a variety of lymphokines that induce B cell maturation and T cell proliferation, and activate macrophages, which kill target cells.

Leukocytes have a fundamental role in the inflammatory and immune response, and include monocytes/macrophages, mast cells, polymorphonucleoleukocytes, natural killer cells, neutrophils, eosinophils, basophils, and myeloid precursors. Leukocyte membrane proteins include members of the CD antigens, N-CAM, I-CAM, human leukocyte antigen (HLA) class I and HLA class II gene products, immunoglobulins, immunoglobulin receptors, complement, complement receptors, interferons, interferon receptors, interleukin receptors, and chemokine receptors.

Abnormal lymphocyte and leukocyte activity has been associated with acute disorders such as AIDS, immune hypersensitivity, leukemias, leukopenia, systemic lupus, granulomatous disease, and eosinophilia.

Apoptosis-Associated Membrane Proteins

A variety of ligands, receptors, enzymes, tumor suppressors, viral gene products, pharmacological agents, and inorganic ions have important positive or negative roles in regulating and implementing the apoptotic destruction of a cell. Although some specific components of the apoptotic pathway have been identified and characterized, many interactions between the proteins involved are undefined, leaving major aspects of the pathway unknown.

A requirement for calcium in apoptosis was previously suggested by studies showing the involvement of calcium levels in DNA cleavage and Fas-mediated cell death (Hewish, D. R. and L. A. Burgoyne (1973) Biochem. Biophys. Res. Comm. 52:504-510; Vignaux, F. et al. (1995) J. Exp. Med. 181:781-786; Oshimi, Y. and S. Miyazaki (1995) J. Immunol. 154:599-609). Other studies show that intracellular calcium concentrations increase when apoptosis is triggered in thymocytes by either T cell receptor cross-linking or by glucocorticoids, and cell death can be prevented by blocking this increase (McConkey, D. J. et al. (1989) J. Immunol. 143:1801-1806; McConkey, D. J. et al. (1989) Arch. Biochem. Biophys. 269:365-370). Therefore, membrane proteins such as calcium channels and the Fas receptor are important for the apopoptic response.

### Transporter-Associated Proteins

Hydrophobic lipid bilayer membranes, highly impermeable to most polar molecules, subdivide organelles into functionally distinct entities. Cells and organelles require transport proteins to import and export essential nutrients and metal ions including K⁺, NH₄⁺, P₁, SO₄²⁻, sugars, and vitamins, as well as various metabolic waste products. Transport proteins also play roles in antibiotic resistance, toxin secretion, ion balance, synaptic neurotransmission, kidney function, intestinal absorption, tumor growth, and other diverse cell functions (Griffith, J. and C. Sansom (1998) The Transporter Facts Book, Academic Press, San Diego Calif., pp. 3-29). Transport can occur by a passive concentration-dependent mechanism, or can be linked to an energy source such as ATP hydrolysis or an ion gradient. Proteins that function in transport include carrier proteins, which bind to a specific solute and undergo a conformational change that translocates the bound solute across the membrane, and channel proteins, which form hydrophilic pores that allow specific solutes to diffuse through the membrane down an electrochemical solute gradient.

Carrier proteins which transport a single solute from one side of the membrane to the other are called uniporters. In contrast, coupled transporters link the transfer of one solute with simultaneous or sequential transfer of a second solute, either in the same direction (symport) or in the opposite direction (antiport). For example, intestinal and kidney epithelium contains a variety of symporter systems driven by the sodium gradient that exists across the plasma membrane. Sodium moves into the cell down its electrochemical gradient and brings the solute into the cell with it. The sodium gradient that provides the driving force for solute uptake is maintained by the ubiquitous Na⁺/K⁺ ATPase system. Sodium-coupled transporters include the mammalian glucose transporter (SGLT1), iodide transporter (NIS), and multivitamin transporter (SMVT). All three transporters have twelve putative transmembrane segments, extracellular glycosylation sites, and cytoplasmically-oriented N- and C-termini. NIS plays a crucial role in the evaluation, diagnosis, and treatment of various thyroid pathologies because it is the molecular basis for radioiodide thyroid-imaging techniques and for specific targeting of radioisotopes to the thyroid gland (Levy, O. et al. (1997) Proc. Natl. Acad. Sci. USA 94:5568-5573). SMVT is expressed in the intestinal mucosa, kidney, and placenta, and is implicated in the transport of the water-soluble vitamins, e.g., biotin and pantothenate (Prasad, P. D. et al. (1998) J. Biol. Chem. 273:7501-7506).

One of the largest families of transporters is the major facilitator superfamily (MFS), also called the uniporter-symporter-antiporter family. MFS transporters are single polypeptide carriers that transport small solutes in response to ion gradients. Members of the MFS are found in all classes of living organisms, and include transporters for sugars, oligosaccharides, phosphates, nitrates, nucleosides, monocarboxylates, and drugs. MFS transporters found in eukaryotes all have a structure comprising 12 transmembrane segments (Pao, S. S. et al. (1998) Microbiol. Molec. Biol. Rev. 62:1-34). The largest family of MPS transporters is the sugar transporter family, which includes the seven glucose transporters (GLUT1-GLUT7) found in humans that are required for the transport of glucose and other hexose sugars. These glucose transport proteins have unique tissue distributions and physiological functions. GLUT1 provides many cell types with their basal glucose requirements and transports glucose across epithelial and endothelial barrier tissues; GLUT2 facilitates glucose uptake or efflux from the liver; GLUT3 regulates glucose supply to neurons; GLUT4 is responsible for insulin-regulated glucose disposal; and GLUT5 regulates fructose uptake into skeletal muscle. Defects in glucose transporters are involved in a recently identified neurological syndrome causing infantile seizures and developmental delay, as well as glycogen storage disease, Fanconi-Bickel syndrome, and non-insulin-dependent diabetes mellitus (Mueckler, M. (1994) Eur. J. Biochem. 219:713-725; Longo, N. and L. J. Elsas (1998) Adv. Pediatr. 45:293-313).

Synip is a novel insulin-regulated syntaxin 4-binding protein which interacts with syntaxin 4, a t-SNARE protein. Insulin-stimulated glucose transport and GLUT4 translocation require regulated interactions between the v-SNARE, VAMP2, and the t-SNARE, syntaxin 4. Data suggests that the Synip:syntaxin 4 complex dissociates because insulin induces a decrease in the binding affinity of Synip for syntaxin 4. In contrast, the carboxyterminal domain of Synip does not dissociate from syntaxin 4 in response to insulin stimulation but rather inhibits glucose transport and GLUT4 translocation (Min, J. et al. (1999) Mol. Cell 3:751-760).

Monocarboxylate anion transporters are proton-coupled symporters with a broad substrate specificity that includes L-lactate, pyruvate, and the ketone bodies acetate, acetoacetate, and beta-hydroxybutyrate. At least seven isoforms have been identified to date. The isoforms are predicted to have twelve transmembrane (TM) helical domains with a large intracellular loop between TM6 and TM7, and play a critical role in maintaining intracellular pH by removing the protons that are produced stoichiometrically with lactate during glycolysis. The best characterized H⁺-monocarboxylate transporter is that of the erythrocyte membrane, which transports L-lactate and a wide range of other aliphatic monocarboxylates. Other cells possess H⁺-linked monocarboxylate transporters with differing substrate and inhibitor selectivities. In particular, cardiac muscle and tumor cells have transporters that differ in their Kₘ values for certain substrates, including stereoselectivity for L- over D-lactate, and in their sensitivity to inhibitors. There are Na⁺-monocarboxylate cotransporters on the luminal surface of intestinal and kidney epithelia, which allow the uptake of lactate, pyruvate, and ketone bodies in these tissues. In addition, there are specific and selective transporters for organic cations and organic anions in organs including the kidney, intestine and liver. Organic anion transporters are selective for hydrophobic, charged molecules with electron-attracting side groups. Organic cation transporters, such as the ammonium transporter, mediate the secretion of a variety of drugs and endogenous metabolites, and contribute to the maintenance of intercellular pH (Poole, R. C. and A. P. Halestrap (1993) Am. J. Physiol. 264: C761-C782; Price, N. T. et al. (1998) Biochei J. 329:321-328; and Martinelle, K. and I. Haggstrom (1993) J. Biotechnol. 30:339-350).

ATP-binding cassette (ABC) transporters, also called the “traffic ATPases”, are a superfamily of membrane proteins that mediate transport and channel functions in prokaryotes and eukaryotes (Higgins, C. P. (1992) Annu. Rev. Cell Biol. 8:67-113). ABC proteins share a similar overall structure and significant sequence homology. All ABC proteins contain a conserved domain of approximately two hundred amino acid residues which includes one or more nucleotide binding domains. Mutations in ABC transporter genes are associated with various disorders, such as hyperbilirubinemia II/Dubin-Johnson syndrome, recessive Stargardt's disease, X-linked adrenoleukodystrophy, multidrug resistance, celiac disease, and cystic fibrosis. ATP-binding cassette (ABC) transporters are members of a superfamily of membrane proteins that transport substances ranging from small molecules such as ions, sugars, amino acids, peptides, and phospholipids, to lipopeptides, large proteins, and complex hydrophobic drugs. ABC transporters consist of four modules: two nucleotide-binding domains (NBD), which hydrolyze ATP to supply the energy required for transport, and two membrane-spanning domains (MSD), each containing six putative transmembrane segments. These four modules may be encoded by a single gene, as is the case for the cystic fibrosis transmembrane regulator (CFTR), or by separate genes. When encoded by separate genes, each gene product contains a single NBD and MSD. These “half-molecules” form homo- and heterodimers, such as Tap1 and Tap2, the endoplasmic reticulum-based major histocompatibility (MHC) peptide transport system. Several genetic diseases are attributed to defects in ABC transporters, such as the following diseases and their corresponding proteins: cystic fibrosis (CFTR, an ion channel), adrenoleukodystrophy (adrenoleukodystrophy protein, ALDP), Zellweger syndrome (peroxisomal membrane protein-70, PMP70), and hyperinsulinemic hypoglycemia (sulfonylurea receptor, SUR). Overexpression of the multidrug resistance (MDR) protein, another ABC transporter, in human cancer cells makes the cells resistant to a variety of cytotoxic drugs used in chemotherapy (Taglicht, D. and S. Michaelis (1998) Meth. Enzymol. 292:130-162).

A number of metal ions such as iron, zinc, copper, cobalt, manganese, molybdenum, selenium, nickel, and chromium are important as cofactors for a number of enzymes. For example, copper is involved in hemoglobin synthesis, connective tissue metabolism, and bone development, by acting as a cofactor in oxidoreductases such as superoxide dismutase, ferroxidase (ceruloplasmin), and lysyl oxidase. Copper and other metal ions must be provided in the diet, and are absorbed by transporters in the gastrointestinal tract. Plasma proteins transport the metal ions to the liver and other target organs, where specific transporters move the ions into cells and cellular organelles as needed. Imbalances in metal ion metabolism have been associated with a number of disease states (Danks, D. M. (1986) J. Med. Genet. 23:99-106).

Transport of fatty acids across the plasma membrane can occur by diffusion, a high capacity, low affinity process. However, under noraal physiological conditions a significant fraction of fatty acid transport appears to occur via a high affinity, low capacity protein-mediated transport process. Fatty acid transport protein (FATP), an integral membrane protein with four transmembrane segments, is expressed in tissues exhibiting high levels of plasma membrane fatty acid flux, such as muscle, heart, and adipose. Expression of FATP is upregulated in 3T3-L1 cells during adipose conversion, and expression in COS7 fibroblasts elevates uptake of long-chain fatty acids (Hui, T. Y. et al. (1998) J. Biol. Chem. 273:27420-27429).

Mitochondrial carrier proteins are transmembrane-spanning proteins which transport ions and charged metabolites between the cytosol and the mitochondrial matrix. Examples include the ADP, ATP carrier protein; the 2-oxoglutaratelmalate carrier; the phosphate carrier protein; the pyruvate carrier; the dicarboxylate carrier which transports malate, succinate, fumarate, and phosphate; the tricarboxylate carrier which transports citrate and malate; and the Grave's disease carrier protein, a protein recognized by IgG in patients with active Grave's disease, an autoimmune disorder resulting in hyperthyroidism. Proteins in this family consist of three tandem repeats of an approximately 100 amino acid domain, each of which contains two transmembrane regions (Stryer, L. (1995) Biochemistry, W.H. Freeman and Company, New York N.Y., p. 551; PROSITE PDOC00189 Mitochondrial energy transfer proteins signature; Online Mendelian Inheritance in Man (OMIM) *275000 Graves Disease).

This class of transporters also includes the mitochondrial uncoupling proteins, which create proton leaks across the inner initochondrial membrane, thus uncoupling oxidative phosphorylation from ATP synthesis. The result is energy dissipation in the form of heat. Mitochondrial uncoupling proteins have been implicated as modulators of thermoregulation and metabolic rate, and have been proposed as potential targets for drugs against metabolic diseases such as obesity (Ricquier, D. et al. (1999) J. Int. Med. 245:637-642).

### Disease Correlation

The etiology of numerous human diseases and disorders can be attributed to defects in the transport of molecules across membranes. Defects in the trafficking of membrane-bound transporters and ion channels are associated with several disorders, e.g., cystic fibrosis, glucose-galactose malabsorption syndrome, hypercholesterolemia, von Gierke disease, and certain forms of diabetes mellitus. Single-gene defect diseases resulting in an inability to transport small molecules across membranes include, e.g., cystinuria, iminoglycinuria, Hartup disease, and Fanconi disease (van't Hoff, W. G. (1996) Exp. Nephrol. 4:253-262; Talente, G. M. et al. (1994) Ann. Intern. Med. 120:218-226; and Chillon, M. et al. (1995) New Engl. J. Med. 332:1475-1480).

Human diseases caused by mutations in ion channel genes include disorders of skeletal muscle, cardiac muscle, and the central nervous system. Mutations in the pore-forming subunits of sodium and chloride channels cause myotonia, a muscle disorder in which relaxation after voluntary contraction is delayed. Sodium channel myotonias have been treated with channel blockers. Mutations in muscle sodium and calcium channels cause forms of periodic paralysis, while mutations in the sarcoplasmic calcium release channel, T-tubule calcium channel, and muscle sodium channel cause malignant hyperthermia Cardiac arrythmia disorders such as the long QT syndromes and idiopathic ventricular fibrillation are caused by mutations in potassium and sodium channels (Cooper, E. C. and L. Y. January (1998) Proc. Natl. Acad. Sci. USA 96:4759-4766). AU four known human idiopathic epilepsy genes code for ion channel proteins (Berkovic, S. F. and I. E. Scheffer (1999) Curr. Opin. Neurology 12:177-182). Other neurological disorders such as ataxias, hemiplegic migraine and hereditary deafness can also result from mutations in ion channel genes (Jen, J. (1999) Curr. Opin. Neurobiol. 9:274-280; Cooper, supra).

Ion channels have been the target for many drug therapies. Neurotransmitter-gated channels have been targeted in therapies for treatment of insomnia, anxiety, depression, and schizophrenia. Voltage-gated channels have been targeted in therapies for arrhythmia, ischemic stroke, head trauma, and neurodegenerative disease (Taylor, C. P. and L. S. Narasimhan (1997) Adv. Pharmacol. 39:47-98). Various classes of ion channels also play an important role in the perception of pain, and thus are potential targets for new analgesics. These include the vanilloid-gated ion channels, which are activated by the vanilloid capsaicin, as well as by noxious heat. Local anesthetics such as lidocaine and mexiletine which blockade voltage-gated Na⁺ channels have been useful in the treatment of neuropathic pain (Eglen, supra).

Ion channels in the immune system have recently been suggested as targets for immunomodulation. T-cell activation depends upon calcium signaling, and a diverse set of T-cell specific ion channels has been characterized that affect this signaling process. Channel blocking agents can inhibit secretion of lymphokines, cell proliferation, and killing of target cells. A peptide antagonist of the T-cell potassium channel Kv1.3 was found to suppress delayed-type hypersensitivity and allogenic responses in pigs, validating the idea of channel blockers as safe and efficacious immunosuppressants (Cahalan, M. D. and K. G. Chandy (1997) Curr. Opin. Biotechnol. 8:749-756).

### Molecules for Disease Detection and Treatment

It is estimated that only 2% of mamalian DNA encodes proteins, and only a small fraction of the genes that encode proteins is actually expressed in a particular cell at any time. The various types of cells in a multicellular organism differ dramatically both in structure and function, and the identity of a particular cell is conferred by its unique pattern of gene expression. In addition, different cell types express overlapping but distinctive sets of genes throughout development. Cell growth and proliferation, cell differentiation, the immune response, apoptosis, and other processes that contribute to organism development and survival are governed by regulation of gene expression. Appropriate gene regulation also ensures that cells function efficiently by expressing only those genes whose functions are required at a given time. Factors that influence gene expression include extracellular signals that mediate cell-cell communication and coordinate the activities of different cell types. Gene expression is regulated at the level of DNA and RNA transcription, and at the level of mRNA translation.

Aberrant expression or mutations in genes and their products may cause, or increase susceptibility to, a variety of human diseases such as cancer and other cell proliferative disorders. The identification of these genes and their products is the basis of an ever-expanding effort to finding markers for early detection of diseases and targets for their prevention and treatment. For example, cancer represents a type of cell proliferative disorder that affects nearly every tissue in the body. The development of cancer, or oncogenesis, is often correlated with the conversion of a normal gene into a cancer-causing gene, or oncogene, through abnormal expression or mutation. Oncoproteins, the products of oncogenes, include a variety of molecules that influence cell proliferation, such as growth factors, growth factor receptors, intracellular signal transducers, nuclear transcription factors, and cell-cycle control proteins. In contrast, tumor-suppressor genes are involved in inhibiting cell proliferation. Mutations which reduce or abrogate the function of tumor-suppressor genes result in aberrant cell proliferation and cancer. Thus a wide variety of genes and their products have been found that are associated with cell proliferative disorders such as cancer, but many more may exist that are yet to be discovered.

DNA-based arrays can provide an efficient, high-throughput method to examine gene expression and genetic variability. For example, SNPs, or single nucleotide polymorphisms, are the most common type of human genetic variation. DNA-based arrays can dramatically accelerate the discovery of SNPs in hundreds and even thousands of genes. Likewise, such arrays can be used for SNP genotyping in which DNA samples from individuals or populations are assayed for the presence of selected SNPs. These approaches will ultimately lead to the systematic identification of all genetic variations in the human genome and the correlation of certain genetic variations with disease susceptibility, responsiveness to drug treatments, and other medically relevant information. (See, for example, Wang, D. G. et al. (1998) Science 280:1077-1082.)

DNA-based array technology is especially important for the rapid analysis of global gene expression patterns. For example, genetic predisposition, disease, or therapeutic treatment may directly or indirectly affect the expression of a large number of genes in a given tissue. In this case, it is useful to develop a profile, or transcript image, of all the genes that are expressed and the levels at which they are expressed in that particular tissue. A profile generated from an individual or population affected with a certain disease or undergoing a particular therapy may be compared with a profile likewise generated from a control individual or population. Such analysis does not require knowledge of gene function, as the expression profiles can be subjected to mathematical analyses which simply treat each gene as a marker. Furthermore, gene expression profiles may help dissect biological pathways by identifying all the genes expressed, for example, at a certain developmental stage, in a particular tissue, or in response to disease or treatment. (See, for example, Lander, E. S. et al. (1996) Science 274:536-539.)

Certain genes are known to be associated with diseases because of their chromosomal location, such as the genes in the myotonic dystrophy (DM) regions of mouse and human. The mutation underlying DM has been localized to a gene encoding the DM-kinase protein, but another active gene, DMR-N9, is in close proximity to the DM-kinase gene (Jansen, G. et al. (1992) Nat. Genet. 1:261-266). DMR-N9 encodes a 650 amino acid protein that contains WD repeats, motifs found in cell signaling proteins. DMR-N9 is expressed in all neural tissues and in the testis, suggesting a role for DMR-N9 in the manifestation of mental and testicular symptoms in severe cases of DM (Jansen, G. et al. (1995) Hum. Mol. Genet. 4:843-852).

Other genes are identified based upon their expression patterns or association with disease syndromes. For example, autoantibodies to subcellular organelles are found in patients with systemic rheumatic diseases. A recently identified protein, golgin-67, belongs to a family of Golgi autoantigens having alpha-helical coiled-coil domains (Eystathioy, T. et al. (2000) J. Autoimmun. 14:179-187). The Stac gene was identified as a brain specific, developmentally regulated gene. The Stac protein contains an SH3 domain, and is thought to be involved in neuron-specific signal transduction (Suzuki, H. et al. (1996) Biochem. Biophys. Res. Commun. 229:902-909).

Ovarian cancer is the leading cause of death from a gynecologic cancer. The majority of ovarian cancers are derived from epithelial cells, and 70% of patients with epithelial ovarian cancers present with late-stage disease. As a result, the long-term survival rate for individuals with this disease is very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns likely vary when normal ovary is compared to ovarian tumors, examination of gene expression in these tissues can identify possible markers for ovarian cancer.

The discovery of new receptors and membrane-associated proteins and the polynucleotides encoding them satisfies a need in the art by providing new compositions which are useful in the diagnosis, prevention, and treatment of cell proliferative, autoimmune/inflammatory, renal, neurological, cardiovascular, metabolic, developmental, endocrine, muscle, gastrointestinal, lipid metabolism, and transport disorders, and viral infections, and in the assessment of the effects of exogenous compounds on the expression of nucleic acid and amino acid sequences of receptors and membrane-associated proteins.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder. For example, both the levels and sequences expressed in tissues from subjects with lung cancer may be compared with the levels and sequences expressed in normal tissue.

The potential application of gene expression profiling is relevant to improving the diagnosis, prognosis, and treatment of cancers, such as lung cancer.

Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium Among the known genes they identified were keratin isoform 6, KOC, SPRC, IGFb2, connexin 26, plakofillin 1 and cytokeratin 13.

There is a need in the art for new compositions, including nucleic acids and proteins, for the diagnosis, prevention, and treatment of cell proliferative, autoimmune/inflamnnatory, renal, neurological, cardiovascular, metabolic, developmental, endocrine, muscle, gastrointestinal, lipid metabolism, and transport disorders, and viral infections.

### SUMMARY OF THE INVENTION

Various embodiments of the invention provide purified polypeptides, receptors and membrane-associated proteins, referred to collectively as “REMAP” and individually as “REMAP-1,” “REMAP-2;” “REMAP-3,” “REMAP-4,” “REMAP-5,” “REMAP-6,” “REMAP-7,” “RMAP-8,” “REMAP-9;” “REMAP-10,” “REMAP-11,” “REMAP-12,” “REMAP-13,” “REMAP-14,” “RMAP-15,” “REMAP-16,” “REMAP-17,” “REMAP-18,” “REMAP-19,” “REMAP-20,” “REMAP-21,” “REMAP-22,” and “REMAP-23,” and methods for using these proteins and their encoding polynucleotides for the detection, diagnosis, and treatment of diseases and medical conditions. Embodiments also provide methods for utilizing the purified receptors and membrane-associated proteins and/or their encoding polynucleotides for facilitating the drug discovery process, including determination of efficacy, dosage, toxicity, and pharmacology. Related embodiments provide methods for utilizing the purified receptors and membrane-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. Another embodiment provides an isolated polypeptide comprising an amino acid sequence of SEQ ID NO:1-23.

Still another embodiment provides an isolated polynucleotide encoding a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. In another embodiment, the polynucleotide encodes a polypeptide selected from the group consisting of SEQ ID NO:1-23. In an alternative embodiment, the polynucleotide is selected from the group consisting of SEQ ID NO:24-46.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23.

Still yet another embodiment provides an isolated polynucleotide selected from the group consisting of a) a polynucleotide comprising a polynucleotide sequence selected from the group consisting of SEQ ID NO:24-46, b) a polynucleotide comprising a naturally occurring polynucleotide sequence at least 90% identical or at least about 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:24-46, b) a polynucleotide comprising a naturally occurring polynucleotide sequence at least 90% identical or at least about 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:24-46, b) a polynucleotide comprising a naturally occurring polynucleotide sequence at least 90% identical or at least about 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23, and a pharmaceutically acceptable excipient. In one embodiment, the composition can comprise an amino acid sequence selected from the group consisting of SEQ ID NO:1-23. Other embodiments provide a method of treating a disease or condition associated with decreased or abnormal expression of functional REMAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional REMAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional REMAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-23, b) a polypeptide comprising a naturally occurring amino acid sequence at least 90% identical or at least about 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:24-46, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:24-46, ii) a polynucleotide comprising a naturally occurring polynucleotide sequence at least 90% identical or at least about 90% identical to a polynucleotide sequence selected from the group consisting of SEQ ID NO:24-46, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:24-46, ii) a polynucleotide comprising a naturally occurring polynucleotide sequence at least 90% identical or at least about 90% identical to a polynucleotide sequence selected from the group consisting of SEQ ID NO:24-46, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### Definitions

“REMAP” refers to the amino acid sequences of substantially purified REMAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of REMAP. Agonists may include proteins, nucleic acids, carbohydrates, small molecules, or any other compound or composition which modulates the activity of REMAP either by directly interacting with REMAP or by acting on components of the biological pathway in which REMAP participates.

An “allelic variant” is an alternative form of the gene encoding REMAP. Allelic variants may result from at least one mutation in the nucleic acid sequence and may result in altered mRNAs or in polypeptides whose structure or function may or may not be altered. A gene may have none, one, or many allelic variants of its naturally occurring form Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding REMAP include those sequences with deletions, insertions, or substitutions of different nucleotides, resulting in a polypeptide the same as REMAP or a polypeptide with at least one functional characteristic of REMAP. Included within this definition are polymorphisms which may or may not be readily detectable using a particular oligonucleotide probe of the polynucleotide encoding REMAP, and improper or unexpected hybridization to allelic variants, with a locus other than the normal chromosomal locus for the polynucleotide encoding REMAP. The encoded protein may also be “altered,” and may contain deletions, insertions, or substitutions of amino acid residues which produce a silent change and result in a functionally equivalent REMAP. Deliberate amino acid substitutions may be made on the basis of one or more similarities in polarity, charge, solubility, hydrophobicity, hydrophilicity, and/or the amphipathic nature of the residues, as long as the biological or immunological activity of REMAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of REMAP. Antagonists may include proteins such as antibodies, anticalins, nucleic acids, carbohydrates, small molecules, or any other compound or composition which modulates the activity of REMAP either by directly interacting with REMAP or by acting on components of the biological pathway in which REMAP participates.

The term “antibody” refers to intact immunoglobulin molecules as well as to fragments thereof, such as Fab, F(ab′)₂, and Fv fragments, which are capable of binding an epitopic determinant. Antibodies that bind REMAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′—OH group of a ribonucleotide may be replaced by 2′—F or 2′—NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “imrmnunologically active” or “immunogenic” refers to the capability of the natural, recombinant, or synthetic REMAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding REMAP or fragments of REMAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of REMAP or a polynucleotide encoding REMAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:24-46 can comprise a region of unique polynucleotide sequence that specifically identifies SEQ ID NO:24-46, for example, as distinct from any other sequence in the genome from which the fragment was obtained. A fragment of SEQ ID NO:24-46 can be employed in one or more embodiments of methods of the invention, for example, in hybridization and amplification technologies and in analogous methods that distinguish SEQ ID NO:24-46 from related polynucleotides. The precise length of a fragment of SEQ ID NO:24-46 and the region of SEQ ID NO:24-46 to which the fragment corresponds are routinely determinable by one of ordinary skill in the art based on the intended purpose for the fragment.

A fragment of SEQ ID NO:1-23 is encoded by a fragment of SEQ ED NO:24-46. A fragment of SEQ ID NO:1-23 can comprise a region of unique amino acid sequence that specifically identifies SEQ ID NO:1-23. For example, a fragment of SEQ ID NO:1-23 can be used as an immunogenic peptide for the development of antibodies that specifically recognize SEQ ID NO:1-23. The precise length of a fragment of SEQ ID NO:1-23 and the region of SEQ ID NO:1-23 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorit Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open: Gap: 5 and Extension Gap: 2 penalties

Gap× drop-off. 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguou nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap× drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be use concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of REMAP which is capable of eliciting an immune response when introduced into a living organism, for example, a mammal. The term “immunogenic fragment” also includes any polypeptide or oligopeptide fragment of REMAP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of REMAP. For example, modulation may cause an increase or a decrease in protein activity, binding characteristics, or any other biological, functional, or immunological properties of REMAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an REMAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of REMAP.

“Probe” refers to nucleic acids encoding REMAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989; Molecular Cloning: A Laboratory Manual, 2^{nd} ed., vol. 1-3, Cold Spring Harbor Press, Plainview N.Y.), Ausubel, F. M. et al. (1999) Short Protocols in Molecular Biology, 4^{th} ed., John Wiley & Sons, New York N.Y.), and Innis, M. et al. (1990; PCR Protocols. A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing REMAP, nucleic acids encoding REMAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 07, 1999) set at default parameters. Such a pair of nucleic acids may show, for example, at least 50%, at least 60%, at least 70%, at least 80%, at least 85%, at least 90%, at least 91%, at least 92%, at lea 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 07, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### The Invention

Various embodiments of the invention include new human receptors and membrane-associated proteins (REMAP), the polynucleotides encoding REMAP, and the use of these compositions for the diagnosis, treatment, or prevention of cell proliferative, autoimmune/inflammatory, renal, neurological, cardiovascular, metabolic, developmental, endocrine, muscle, gastrointestinal, lipid metabolism, and transport disorders, and viral infections.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to polypeptide and polynucleotide embodiments. The full length clones encode polypeptides which have at least 95% sequence identity to the polypeptides shown in column 3.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are receptors and membrane-associated proteins. For example, SEQ ID NO:1 is 46% identical, from residue I108 to residue P348, to Gallus gallus ChT1 (GenBank ID g433593) as determined by the Basic Local Alignment Search Tool (BLAST). (See Table 2.) The BLAST probability score is 1.0e-70, which indicates the probability of obtaining the observed polypeptide sequence alignment by chance. SEQ ID NO:1 also contains immunoglobulin domains, as determined by searching for statistically significant matches in the hidden Markov model (HMM)-based PFAM database of conserved protein family domains. (See Table 3.) Data additional BLAST analyses provide further corroborative evidence that SEQ ID NO:1 is a ChT1 homolog (note that ChT1 is a member of an immunoglobulin superfamily). In an alternative example, SEQ ID NO:3 is 87% identical, from residue M562 to residue C641, to epidermal growth factor receptor-related protein (GenBank ID g178252) as determined by the Basic Local Alignment Search Tool (BLAST). (See Table 2.) The BLAST probability score is 5.0e-38, which indicates the probability of obtaining the observed polypeptide sequence alignment by chance. SEQ ID NO:3 also contains a rhomboid family domain as determined by searching for statistically significant matches in the hidden Markov model (HMM)-based PFAM database of conserved protein family domains. (See Table 3.) Data from TMHMMER analysis provide further corroborative evidence that SEQ ID NO:3 is an integral membrane protein, particulary an epidermal growth factor receptor-related protein. In an alternative example, SEQ ID NO:5 is 93% identical, from residue M1 to residue I1168, to human SorCSb, a splice variant of the VPS10 domain receptor SorCS (GenBank ID g7715916) as determined by the Basic Local Alignment Search Tool (BLAST). (See Table 2.) The BLAST probability score is 0.0, which indicates the probability of obtaining the observed polypeptide sequence alignment by chance. SEQ ID NO:5 also contains a BNR repeat and a PKD domain as determined by searching for statistically significant matches in the hidden Markov model (HMM)-based PFAM database of conserved protein family domains. (See Table 3.) Data from BLAST_PRODOM analyses provide further corroborative evidence that SEQ ID NO:5 is a VPS10-containing receptor. In an alternative example, SEQ ID NO:7 is 38% identical, from residue S2 to residue N232, to human MS4A8B protein (GenBank ID g13649390) as determined by the Basic Local Alignment Search Tool (BLAST). (See Table 2.) The BLAST probability score is 1.2e-28, which indicates the probability of obtaining the observed polypeptide sequence alignment by chance. MS4A8B is a member of a family of proteins related to the B-cell-specific antigen CD20, a hematopoietic-cell-specific protein HTm4, and high affinity IgE receptor beta chain (FcvarepsilonRIbeta). All family members have at least four potential membrane-spanning domains, with N- and C-terminal cytoplasmic domains, hence the name membrane-spanning 4A gene family (Liang et al. (2001) Genomics 72 (2), 119-127). Data from MOTIFS and further BLAST analyses provide corroborative evidence that SEQ ID NO:7 is a membrane-associated protein. In an alternative example, SEQ ID NO:10 is 30% identical, from residue T27 to residue N304, to rat neuropilin-2 (GenBank ID g2367641) as determined by the Basic Local Alignment Search Tool (BLAST). (See Table 2.) The BLAST probability score is 2.9e-23, which indicates the probability of obtaining the observed polypeptide sequence alignment by chance. SEQ ID NO:10 also contains CUB extracellular domains and a low-density lipoprotein receptor domain as determined by searching for statistically significant matches in the hidden Markov model (HMM)-based PFAM database of conserved protein family domains. Data from BLOCKS and additional BLAST analysis also support the identification (See Table 3.) In an alternative example, For example, SEQ ID NO:11 is 91% identical, from residue M1 to residue A2214, to rat Munc 13-3 (GenBank ID g1763306) as determined by the Basic Local Alignment Search Tool (BLAST). (See Table 2.) The BLAST probability score is 0.0, which indicates the probability of obtaining the observed polypeptide sequence alignment by chance. SEQ ID NO:11 also contains C2 and phorbol esters/diacylglycerol binding (C1) domains as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:11 is a protein involved in membrane trafficking. In an alternative example, SEQ ID NO:13 is 60% identical, from residue M1 to residue S381, to Synip, a mouse syntaxin 4-interacting protein (GenBank ID g5453324) as determined by the Basic Local Alignment Search Tool (BLAST). (See Table 2.) The BLAST probability score is 3.1e-112, which indicates the probability of obtaining the observed polypeptide sequence alignment by chance. SEQ ID NO:13 also contains a PDZ (DHR or GLGF) domain as determined by searching for statistically significant matches in the hidden Markov model (HMM)-based PFAM database of conserved protein family domains. (See Table 3.) Data from BLIPS and other BLAST analyses provide further corroborative evidence that SEQ ID NO:13 is a syntaxin 4-interacting protein. In an alternative example, SEQ ID NO:15 is 99% identical, from residue L15 to residue L327, to CD68, a human transmembrane glycoprotein (GenBank ID g298665) as determined by the Basic Local Alignment Search Tool (BLAST). (See Table 2.) The BLAST probability score is 4.4e-168, which indicates the probability of obtaining the observed polypeptide sequence alignment by chance. SEQ ID NO:15 also contains a human lysosome-associated membrane glycoprotein (Lamp) domain as determined by searching for statistically significant matches in the hidden Markov model (HMM)-based PFAM database of conserved protein family domains. (See Table 3.) Data from BLIMPS, MOTIFS, and other BLAST analyses provide further corroborative evidence that SEQ ID NO:15 is a transmembrane glycoprotein. SEQ ID NO:2, SEQ ID NO:4, SEQ ID NO:6, SEQ ID NO:8-9, SEQ ID NO:12, SEQ ID NO:14, and SEQ ID NO:16-23 were analyzed and annotated in a similar manner. The algorithms and parameters for the analysis of SEQ ID NO:1-23 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:24-46 or that distinguish between SEQ ID NO:24-46 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and AN_{1,2,3 . . .} , if present, represent specific exons that may have been manually edited during analysis (See Example V). Alternatively, the polynucleotide fragments in column 2 may refer to assemblages of exons brought together by an “exon-stretching” algorithm For example, a polynucleotide sequence identified as FLXXXXXX_gAAAAA_{—gBBBBB}_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| | Type of analysis and/or |
|---|---|
| Prefix | examples of programs |
| GNN, GFG, ENST | Exon prediction from genomic |
| | sequences using, for example, |
| | GENSCAN (Stanford University, |
| | CA, USA) or FGENES (Computer |
| | Genomics Group, The Sanger Centre, |
| | Cambridge, UK). |
| GBI | Hand-edited analysis of |
| | genomic sequences. |
| FL | Stitched or stretched genomic |
| | sequences (see Example V). |
| INCY | Full length transcript and exon |
| | prediction from mapping of EST |
| | sequences to the genome. Genomic |
| | location and EST composition |
| | data are combined to predict |
| | the exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses REMAP variants. A preferred REMAP variant is one which has at least about 80%, or alternatively at least about 90%, or even at least about 95% amino acid sequence identity to the REMAP amino acid sequence, and which contains at least one functional or structural characteristic of REMAP.

Various embodiments also encompass polynucleotides which encode REMAP. In a particular embodiment, the invention encompasses a polynucleotide sequence comprising a sequence selected from the group consisting of SEQ ID NO:24-46, which encodes REMAP. The polynucleotide sequences of SEQ ID NO:24-46, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding REMAP. In particular, such a variant polynucleotide will have at least about 70%, or alternatively at least about 85%, or even at least about 95% polynucleotide sequence identity to a polynucleotide encoding REMAP. A particular aspect of the invention encompasses a variant of a polynucleotide comprising a sequence selected from the group consisting of SEQ ID NO:24-46 which has at least about 70%, or alternatively at least about 85%, or even at least about 95% polynucleotide sequence identity to a nucleic acid sequence selected from the group consisting of SEQ ID NO:24-46. Any one of the polynucleotide variants described above can encode a polypeptide which contains at least one functional or structural characteristic of REMAP.

In addition, or in the alternative, a polynucleotide variant of the invention is a splice variant of a polynucleotide encoding REMAP. A splice variant may have portions which have significant sequence identity to a polynucleotide encoding REMAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding REMAP over its entire length; however, portions of the splice variant will have at least about 70%, or alternatively at least about 85%, or alternatively at least about 95%, or alternatively 100% polynucleotide sequence identity to portions of the polynucleotide encoding REMAP. For example, a polynucleotide comprising a sequence of SEQ ID NO:30 and a polynucleotide comprising a sequence of SEQ ID NO:46 are splice variants of each other; and a polynucleotide comprising a sequence of SEQ ID NO:31 and a polynucleotide comprising a sequence of SEQ ID NO:32 are splice variants of each other. Any one of the splice variants described above can encode a polypeptide which contains at least one functional or structural characteristic of REMAP.

It will be appreciated by those skilled in the art that as a result of the degeneracy of the genetic code, a multitude of polynucleotide sequences encoding REMAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring REMAP, and all such variations are to be considered as being specifically disclosed.

Although polynucleotides which encode REMAP and its variants are generally capable of hybridizing to polynucleotides encoding naturally occurring REMAP under appropriately selected conditions of stringency, it may be advantageous to produce polynucleotides encoding REMAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding REMAP and its derivatives without altering the encoded amino acid sequences include the production of RNA transcripts having more desirable properties, such as a greater half-life, than transcripts produced from the naturally occurring sequence.

The invention also encompasses production of polynucleotides which encode REMAP and REMAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding REMAP or any fragment thereof.

Embodiments of the invention can also include polynucleotides that are capable of hybridizing to the claimed polynucleotides, and, in particular, to those having the sequences shown in SEQ ID NO:24-46 and fragments thereof, under various conditions of stringency (Wahl, G. M. and S. L. Berger (1987) Methods Enzymol. 152:399407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding REMAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode REMAP may be cloned in recombinant DNA molecules that direct expression of REMAP, or fragments or functional equivalents thereof, in appropriate host cells. Due to the inherent degeneracy of the genetic code, other polynucleotides which encode substantially the same or a functionally equivalent polypeptides may be produced and used to express REMAP.

The polynucleotides of the invention can be engineered using methods generally known in the art in order to alter REMAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat Biotechnol. 17:259-264; and Crameri, A. et al. (1996) Nat. Biotechnol. 14:315-319) to alter or improve the biological properties of REMAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding REMAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, REMAP itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of REMAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing. (Creighton, supra, pp. 28-53).

In order to express a biologically active REMAP, the polynucleotides encoding REMAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding REMAP. Such elements may vary in their strength and specificity. Specific initiation signals may also be used to achieve more efficient translation of polynucleotides encoding REMAP. Such signals include the ATG initiation codon and adjacent sequences, e.g. the Kozak sequence. In cases where a polynucleotide sequence encoding REMAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding REMAP and appropriate transcriptional and translational control elements. These methods include in vitro recombinant DNA techniques, synthetic techniques, and in vivo genetic recombination (Sambrook, J. et al. (1989) Molecular Cloning, A Laboratory Manual, Cold Spring Harbor Press, Plainview N.Y., ch. 4, 8, and 16-17; Ausubel et al., supra, ch. 1, 3, and 15).

A variety of expression vector/host systems may be utilized to contain and express polynucleotides encoding REMAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology(1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding REMAP. For example, routine cloning, subcloning, and propagation of polynucleotides encoding REMAP can be achieved using a multifunctional E. coli vector such as PBLUESCRIPT (Stratagene, La Jolla Calif.) or PSPORT1 plasmid (Invitrogen). Ligation of polynucleotides encoding REMAP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of REMAP are needed, e.g. for the production of antibodies, vectors which direct high level expression of REMAP may be used. For example, vectors containing the strong, inducible SP6 or T7 bacteriophage promoter may be used.

Yeast expression systems may be used for production of REMAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of REMAP. Transcription of polynucleotides encoding REMAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mamnmalian cells, a number of viral-based expression systems may be utilized. In cases where an adenovirus is used as an expression vector, polynucleotides encoding REMAP may be ligated into an adenovirus transcription/translation complex consisting of the late promoter and tripartite leader sequence. Insertion in a non-essential E1 or E3 region of the viral genome may be used to obtain infective virus which expresses REMAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of REMAP in cell lines is preferred. For example, polynucleotides encoding REMAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thynidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding REMAP is inserted within a marker gene sequence, transformed cells containing polynucleotides encoding REMAP can be identified by the absence of marker gene function. Alternatively, a marker gene can be placed in tandem with a sequence encoding REMAP under the control of a single promoter. Expression of the marker gene in response to induction or selection usually indicates expression of the tandem gene as well.

In general, host cells that contain the polynucleotide encoding REMAP and that express REMAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of REMAP using either specific polyclonal or monoclonal antibodies are known in the art. Examples of such techniques include enzyme-linked immunosorbent assays (ELISAs), radioimmunoassays (RIAs), and fluorescence activated cell sorting (SACS). A two-site, monoclonal-based immunoassay utilizing monoclonal antibodies reactive to two non-interfering epitopes on REMAP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding REMAP include oligolabeling, nick translation, end-labeling, or PCR amplification using a labeled nucleotide. Alternatively, polynucleotides encoding REMAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding REMAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode REMAP may be designed to contain signal sequences which direct secretion of REMAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding REMAP may be ligated to a heterologous sequence resulting in translation of a fusion protein in any of the aforementioned host systems. For example, a chimeric REMAP protein containing a heterologous moiety that can be recognized by a commercially available antibody may facilitate the screening of peptide libraries for inhibitors of REMAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the REMAP encoding sequence and the heterologous protein sequence, so that REMAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled REMAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

REMAP, fragments of REMAP, or variants of REMAP may be used to screen for compounds that specifically bind to REMAP. One or more test compounds may be screened for specific binding to REMAP. In various embodiments, 1, 2, 3, 4, 5, 10, 20, 50, 100, or 200 test compounds can be screened for specific binding to REMAP. Examples of test compounds can include antibodies, anticalins, oligonucleotides, proteins (e.g., ligands or receptors), or small molecules.

In related embodiments, variants of REMAP can be used to screen for binding of test compounds, such as antibodies, to REMAP, a variant of REMAP, or a combination of REMAP and/or one or more variants REMAP. In an embodiment, a variant of REMAP can be used to screen for compounds that bind to a variant of REMAP, but not to REMAP having the exact sequence of a sequence of SEQ ID NO:1-23. REMAP variants used to perform such screening can have a range of about 50% to about 99% sequence identity to REMAP, with various embodiments having 60%, 70%, 75%, 80%, 85%, 90%, and 95% sequence identity.

In an embodiment, a compound identified in a screen for specific binding to REMAP can be closely related to the natural ligand of REMAP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor REMAP (Howard, A. D. et al. (2001) Trends Pharmacol. Sci.22: 132-140; Wise, A. et al. (2002) Drug Discovery Today 7:235-246).

In other embodiments, a compound identified in a screen for specific binding to REMAP can be closely related to the natural receptor to which REMAP binds, at least a fragment of the receptor, or a fragment of the receptor including all or a portion of the ligand binding site or binding pocket. For example, the compound may be a receptor for REMAP which is capable of propagating a signal, or a decoy receptor for REMAP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Immunex Corp., Seattle Wash.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to REMAP, fragments of REMAP, or variants of REMAP. The binding specificity of the antibodies thus screened can thereby be selected to identify particular fragments or variants of REMAP. In one embodiment, an antibody can be selected such that its binding specificity allows for preferential identification of specific fragments or variants of REMAP. In another embodiment, an antibody can be selected such that its binding specificity allows for preferential diagnosis of a specific disease or condition having increased, decreased, or otherwise abnormal production of REMAP.

In an embodiment, anticalins can be screened for specific binding to REMAP, fragments of REMAP, or variants of REMAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities; The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit REMAP involves producing appropriate cells which express REMAP, either as a secreted protein or on the cell membrane. Preferred cells include cells from mammals, yeast, Drosophila, or E. coli. Cells expressing REMAP or cell membrane fractions which contain REMAP are then contacted with a test compound and binding, stimulation, or inhibition of activity of either REMAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with REMAP, either in solution or affixed to a solid support, and detecting the binding of REMAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

REMAP, fragments of REMAP, or variants of REMAP may be used to screen for compounds that modulate the activity of REMAP. Such compounds may include agonists, antagonists, or partial or inverse agonists. In one embodiment, an assay is performed under conditions permissive for REMAP activity, wherein REMAP is combined with at least one test compound, and the activity of REMAP in the presence of a test compound is compared with the activity of REMAP in the absence of the test compound. A change in the activity of REMAP in the presence of the test compound is indicative of a compound that modulates the activity of REMAP. Alternatively, a test compound is combined with an in vitro or cell-free system comprising REMAP under conditions suitable for REMAP activity, and the assay is performed. In either of these assays, a test compound which modulates the activity of REMAP may do so indirectly and need not come in direct contact with the test compound. At least one and up to a plurality of test compounds may be screened.

In another embodiment, polynucleotides encoding REMAP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding REMAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding REMAP can also be used to create “knockin” humanized animals (pigs) or transgenic animals (mice or rats) to model human disease. With knockin technology, a region of a polynucleotide encoding REMAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress REMAP, e.g., by secreting REMAP in its milk, may also serve as a convenient source of that protein (Janne, J. et al. (1998) Biotechnol. Annu. Rev. 4:55-74).

### Therapeutics

Chemical and structural similarity, e.g., in the context of sequences and motifs, exists between regions of REMAP and receptors and membrane-associated proteins. In addition, examples of tissues expressing REMAP can be found in Table 6 and can also be found in Example XI. Therefore, REMAP appears to play a role in cell proliferative, autoimmune/inflammatory, renal, neurological, cardiovascular, metabolic, developmental, endocrine, muscle, gastrointestinal, lipid metabolism, and transport disorders, and viral infections. In the treatment of disorders associated with increased REMAP expression or activity, it is desirable to decrease the expression or activity of REMAP. In the treatment of disorders associated with decreased REMAP expression or activity, it is desirable to increase the expression or activity of REMAP.

Therefore, in one embodiment, REMAP or a fragment or derivative thereof may be administered to a subject to treat or prevent a disorder associated with decreased expression or activity of REMAP. Examples of such disorders include, but are not limited to, Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fingal, parasitic, protozoal, and helminthic infections, and trauma; a renal disorder such as renal amyloidosis, hypertension, primary aldosteronism, Addison's disease, renal failure, glomerulonephritis, chronic glomerulonephritis, tubulointerstitial nephritis, a cystic disorder of the kidney, a dysplastic malformation such as polycystic disease, renal dysplasias, and cortical or medullary cysts, an inherited polycystic renal disease (PRD), such as recessive and autosomal dominant PRD, medullary cystic disease, medullary sponge kidney and tubular dysplasia, Alport's syndrome, a non-renal cancer which affects renal physiology, such as a bronchogenic tumor of the lung or a tumor of the basal region of the brain, multiple myeloma, an adenocarcinoma of the kidney, metastatic renal carcinoma, any functional or morphologic change in the kidney produced by any pharmaceutical, chemical, or biological agent ingested, injected, inhaled, or absorbed such as a heavy metal, an antibiotic, an analgesic, a solvent, an oxalosis-inducing agent, an anticancer drug, a herbicide, and an antiepileptic; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a cardiovascular disorder such as arteriovenous fistula, atherosclerosis, hypertension, vasculitis, Raynaud's disease, aneurysms, arterial dissections, varicose veins, thrombophlebitis and phlebothrombosis, vascular tumors, complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; a metabolic disorder such as Addison's disease, cerebrotendinous xanthomatosis, congenital adrenal hyperplasia, coumarin resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudovitamin D-deficiency rickets, disorders of carbohydrate metabolism such as congenital type II dyserythropoietic anemia, diabetes, insulin-dependent diabetes mellitus, non-insulin-dependent diabetes mellitus, galactose epimerase deficiency, glycaogen storage diseases, lysosomal storage diseases, fructosuria, pentosuria, and inherited abnormalities of pyruvate metabolism, disorders of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type IX diabetes; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, a seizure disorder such as Syndenham's chorea and cerebral palsy, spina bifida, anencephaly, craniorachischisis, congenital glaucoma, cataract, and sensorineural hearing loss; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kaliman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SIADH) often caused by benign adenoma, a disorder associated with hypothyroidism including goiter, myxedema, acute thyroiditis associated with bacterial infection, subacute thyroiditis associated with viral infection, autoimmune thyroiditis (Hashinoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia; a muscle disorder such as Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, ethanol myopathy, angina, anaphylactic shock, arrhythmias, asthma, cardiovascular shock, Cushing's syndrome, hypertension, hypoglycemia, myocardial infarction, migraine, pheochromocytoma, and myopathies including encephalopathy, epilepsy, Kearns-Sayre syndrome, lactic acidosis, myoclonic disorder, ophthalmoplegia, and acid maltase deficiency (AMD, also known as Pompe's disease); a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a disorder of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoffs disease, hyperlipidemia, hyperlipemia, lipid myopathies, and obesity; and a transport disorder such as akinesia, amyotrophic lateral sclerosis, ataxia telangiectasia, cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthermia, multidrug resistance, myasthenia gravis, myotonic dystrophy, catatonia, tardive dyskinesia, dystonias, peripheral neuropathy, cerebral neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulnonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; and a viral infection such as those caused by adenoviruses (acute respiratory disease, pneumonia), arenaviruses (lymphocytic choriomeningitis), bunyaviruses (Hantavirus), coronaviruses (pneumonia, chronic bronchitis), hepadnaviruses (hepatitis), herpesviruses (herpes simplex virus, varicella-zoster virus, Epstein-Barr virus, cytomegalovirus), flaviviruses (yellow fever), orthomyxoviruses (influenza), papillomaviruses (cancer), paramyxoviruses (measles, mumps), picomoviruses (rhinovirus, poliovirus, coxsackie-virus), polyomaviruses (BK virus, JC virus), poxviruses (smallpox), reovirus (Colorado tick fever), retroviruses (human immunodeficiency virus, human T lymphotropic virus), rhabdoviruses (rabies), rotaviruses (gastroenteritis), and togaviruses (encephalitis, rubella).

In another embodiment, a vector capable of expressing REMAP or a fragment or derivative thereof may be administered to a subject to treat or prevent a disorder associated with decreased expression or activity of REMAP including, but not limited to, those described above.

In a further embodiment, a composition comprising a substantially purified REMAP in conjunction with a suitable pharmaceutical carrier may be administered to a subject to treat or prevent a disorder associated with decreased expression or activity of REMAP including, but not limited to, those provided above.

In still another embodiment, an agonist which modulates the activity of REMAP may be administered to a subject to treat or prevent a disorder associated with decreased expression or activity of REMAP including, but not limited to, those listed above.

In a further embodiment, an antagonist of REMAP may be administered to a subject to treat or prevent a disorder associated with increased expression or activity of REMAP. Examples of such disorders include, but are not limited to, those cell proliferative, autoimmune/inflammatory, renal, neurological, cardiovascular, metabolic, developmental, endocrine, muscle, gastrointestinal, lipid metabolism, and transport disorders, and viral infections described above. In one aspect, an antibody which specifically binds REMAP may be used directly as an antagonist or indirectly as a targeting or delivery mechanism for bringing a pharmaceutical agent to cells or tissues which express REMAP.

In an additional embodiment, a vector expressing the complement of the polynucleotide encoding REMAP may be administered to a subject to treat or prevent a disorder associated with increased expression or activity of REMAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of REMAP may be produced using methods which are generally known in the art. In particular, purified REMAP may be used to produce antibodies or to screen libraries of pharmaceutical agents to identify those which specifically bind REMAP. Antibodies to REMAP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with REMAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to REMAP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of REMAP amino acids may be fused with those of another protein, such as KLH, and antibodies to the chimeric molecule may be produced.

Monoclonal antibodies to REMAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:4524-54). Alternatively, techniques described for the production of single chain antibodies may be adapted, using methods known in the art, to produce REMAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for REMAP may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between REMAP and its specific antibody. A two-site, monoclonal-based immunoassay utilizing monoclonal antibodies reactive to two non-interfering REMAP epitopes is generally used, but a competitive binding assay may also be employed (Pound, supra).

Various methods such as Scatchard analysis in conjunction with radioimmunoassay techniques may be used to assess the affinity of antibodies for REALP. Affinity is expressed as an association constant, Kₐ, which is defined as the molar concentration of REMAP-antibody complex divided by the molar concentrations of free antigen and free antibody under equilibrium conditions. The Kₐ determined for a preparation of polyclonal antibodies, which are heterogeneous in their affinities for multiple REMAP epitopes, represents the average affinity, or avidity, of the antibodies for REMAP. The Kₐ determined for a preparation of monoclonal antibodies, which are monospecific for a particular REMAP epitope, represents a true measure of affinity. High-affinity antibody preparations with Kₐ ranging from about 10⁹ to 10¹² L/mole are preferred for use in immunoassays in which the REMAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of REMAP, preferably in active form, from the antibody (Catty, D. (1988) Antibodies. Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of REMAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding REMAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding REMAP. Such technology is well known in the art, and antisense oligonucleotides or larger fragments can be designed from various locations along the coding or control regions of sequences encoding REMAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding REMAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine dearinase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in REMAP expression or regulation causes disease, the expression of REMAP from an appropriate population of transduced cells may alleviate the clinical manifestations caused by the genetic deficiency.

In a further embodiment of the invention, diseases or disorders caused by deficiencies in REMAP are treated by constructing mammalian expression vectors encoding REMAP and introducing these vectors by mechanical means into REMAP-deficient cells. Mechanical transfer technologies for use with cells iii vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Recipon (1998) Curr. Opin. Biotechnol. 9:445-450).

Expression vectors that may be effective for the expression of REMAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). REMAP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding REMAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to REMAP expression are treated by constructing a retrovirus vector consisting of (i) the polynucleotide encoding REMAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, L. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding REMAP to cells which have one or more genetic abnormalities with respect to the expression of REMAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding REMAP to target cells which have one or more genetic abnormalities with respect to the expression of REMAP. The use of herpes simplex virus (HSV)-based vectors may be especially valuable for introducing REMAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation-of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding REMAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for RBMAP into the alphavirus genome in place of the capsid-coding region results in the production of a large number of REMAP-coding RNAs and the synthesis of high levels of REMAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of REMAP into a variety of cell types. The specific transduction of a subset of cells in a population may require the sorting of cells prior to taansduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions -10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding REMAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding REMAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding REMAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased REMAP expression or activity, a compound which specifically inhibits expression of the polynucleotide encoding REMAP may be therapeutically useful, and in the treatment of disorders associated with decreased REMAP expression or activity, a compound which specifically promotes expression of the polynucleotide encoding REMAP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding REMAP is exposed to at least one test compound thus obtained. The sample may comprise, for example, an intact or permeabilized cell, or an in vitro cell-free or reconstituted biochemical system. Alterations in the expression of a polynucleotide encoding REMAP are assayed by any method commonly known in the art. Typically, the expression of a specific nucleotide is detected by hybridization with a probe having a nucleotide sequence complementary to the sequence of the polynucleotide encoding REMAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28: E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of REMAP, antibodies to REMAP, and mimetics, agonists, antagonists, or inhibitors of REMAP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising REMAP or fragments thereof. For example, liposome preparations containing a cell-impermeable macromolecule may promote cell fusion and intracellular delivery of the macromolecule. Alternatively, REMAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example REMAP or fragments thereof, antibodies of REMAP, and agonists, antagonists or inhibitors of REMAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhlbitors. Similarly, delivery of polynucleotides or polypeptides will be specific to particular cells, conditions, locations, etc.

### Diagnostics

In another embodiment, antibodies which specifically bind REMAP may be used for the diagnosis of disorders characterized by expression of REMAP, or in assays to monitor patients being treated with REMAP or agonists, antagonists, or inhibitors of REMAP. Antibodies useful for diagnostic purposes may be prepared in the same manner as described above for therapeutics. Diagnostic assays for REMAP include methods which utilize the antibody and a label to detect REMAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring REMAP, including ELISAs, RIAs, and FACS, are known in the art and provide a basis for diagnosing altered or abnormal levels of REMAP expression. Normal or standard values for REMAP expression are established by combining body fluids or cell extracts taken from normal mammalian subjects, for example, human subjects, with antibodies to REMAP under conditions suitable for complex formation. The amount of standard complex formation may be quantitated by various methods, such as photometric means. Quantities of REMAP expressed in subject, control, and disease samples from biopsied tissues are compared with the standard values. Deviation between standard and subject values establishes the parameters for diagnosing disease.

In another embodiment of the invention, polynucleotides encoding REMAP may be used for diagnostic purposes. The polynucleotides which may be used include oligonucleotides, complementary RNA and DNA molecules, and PNAs. The polynucleotides may be used to detect and quantify gene expression in biopsied tissues in which expression of REMAP may be correlated with disease. The diagnostic assay may be used to determine absence, presence, and excess expression of REMAP, and to monitor regulation of REMAP levels during therapeutic intervention.

In one aspect, hybridization with PCR probes which are capable of detecting polynucleotides, including genomic sequences, encoding REMAP or closely related molecules may be used to identify nucleic acid sequences which encode REMAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding REMAP, allelic variants, or related sequences.

Probes may also be used for the detection of related sequences, and may have at least 50% sequence identity to any of the REMAP encoding sequences. The hybridization probes of the subject invention may be DNA or RNA and may be derived from the sequence of SEQ ID NO:24-46 or from genomic sequences including promoters, enhancers, and introns of the REMAP gene.

Means for producing specific hybridization probes for polynucleotides encoding REMAP include the cloning of polynucleotides encoding REMAP or REMAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidinibiotin coupling systems, and-the like.

Polynucleotides encoding REMAP may be used for the diagnosis of disorders associated with expression of REMAP. Examples of such disorders include, but are not limited to, Examples of such disorders include, but are not limited to, a cell proliferative disorder such as actinic keratosis, arteriosclerosis, atherosclerosis, bursitis, cirrhosis, hepatitis, mixed connective tissue disease (MCTD),-myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflamnimatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid artritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a renal disorder such as renal amyloidosis, hypertension, primary aldosteronism, Addison's disease, renal failure, glomerulonephritis, chronic glomerulonephritis, tubulointerstitial nephritis, a cystic disorder of the kidney, a dysplastic malformation such as polycystic disease, renal dysplasias, and cortical or medullary cysts, an inherited polycystic renal disease (PRD), such as recessive and autosomal dominant PRD, medullary cystic disease, medullary sponge kidney and tubular dysplasia, Alport's syndrome, a non-renal cancer which affects renal physiology, such as a bronchogenic tumor of the lung or a tumor of the basal region of the brain, multiple myeloma, an adenocarcinoma of the kidney, metastatic renal carcinoma, any functional or morphologic change in the kidney produced by any pharmaceutical, chemical, or biological agent ingested, injected, inhaled, or absorbed such as a heavy metal, an antibiotic, an analgesic, a solvent, an oxalosis-inducing agent, an anticancer drug, a herbicide, and an antiepileptic; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a cardiovascular disorder such as arteriovenous fistula, atherosclerosis, hypertension, vasculitis, Raynaud's disease, aneurysms, arterial dissections, varicose veins, thrombophlebitis and phlebothrombosis, vascular tumors, complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; a metabolic disorder such as Addison's disease, cerebrotendinous xanthomatosis, congenital adrenal hyperplasia, coumain resistance, cystic fibrosis, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, osteoporosis, phenylketonuria, pseudovitamin D-deficiency rickets, disorders of carbohydrate metabolism such as congenital type II dyserythropoietic anemia, diabetes, insulin-dependent diabetes mellitus, non-insulin-dependent diabetes mellitus, galactose epimerase deficiency, glycogen storage diseases, lysosomal storage diseases, fructosuria, pentosuria, and inherited abnormalities of pyruvate metabolism, disorders of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease; Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, and lipid myopathies, and disorders of copper metabolism such as Menke's disease, Wilson's disease, and Ehlers-Danlos syndrome type IX diabetes; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, a seizure disorder such as Syndenham's chorea and cerebral palsy, spina bifida, anencephaly, craniorachischisis, congenital glaucoma, cataract, and sensorineural hearing loss; and an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma, a disorder associated with hypopituitarism including hypogonadism, Sheehan syndrome, diabetes insipidus, Kailman's disease, Hand-Schuller-Christian disease, Letterer-Siwe disease, sarcoidosis, empty sella syndrome, and dwarfism, a disorder associated with hyperpituitarism including acromegaly, giantism, and syndrome of inappropriate antidiuretic hormone (ADH) secretion (SIADH) often caused by benign adenoma, a disorder associated with hypothyroidism including goiter, myxedema, acute thyroiditis associated with bacterial infection, subacute thyroiditis associated with viral infection, autoimmune thyroiditis (Hashimoto's disease), and cretinism, a disorder associated with hyperthyroidism including thyrotoxicosis and its various forms, Grave's disease, pretibial myxedema, toxic multinodular goiter, thyroid carcinoma, and Plummer's disease, a disorder associated with hyperparathyroidism including Conn disease (chronic hypercalemia), a pancreatic disorder such as Type I or Type II diabetes mellitus and associated complications, a disorder associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease, a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis, and, in men, Leydig cell deficiency, male climacteric phase, and germinal cell aplasia, a hypergonadal disorder associated with Leydig cell tumors, androgen resistance associated with absence of androgen receptors, syndrome of 5 α-reductase, and gynecomastia; a muscle disorder such as Duchenne's muscular dystrophy, Becker's muscular dystrophy, myotonic dystrophy, central core disease, nemaline myopathy, centronuclear myopathy, lipid myopathy, mitochondrial myopathy, infectious myositis, polymyositis, dermatomyositis, inclusion body myositis, thyrotoxic myopathy, ethanol myopathy, angina, anaphylactic shock, arrhythmias, asthma, cardiovascular shock, Cushing's syndrome, hypertension, hypoglycemia, myocardial infarction, migraine, pheochromocytoma, and myopathies including encephalopathy, epilepsy, Kearns-Sayre syndrome, lactic acidosis, myoclonic disorder, ophthalmoplegia, and acid maltase deficiency (AMD, also known as Pompe's disease); a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a disorder of lipid metabolism such as fatty liver, cholestasis, primary biliary cirrhosis, carnitine deficiency, carnitine palritoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity; and a transport disorder such as akinesia, amyotrophic lateral sclerosis, ataxia telangiectasiai cystic fibrosis, Becker's muscular dystrophy, Bell's palsy, Charcot-Marie Tooth disease, diabetes mellitus, diabetes insipidus, diabetic neuropathy, Duchenne muscular dystrophy, hyperkalemic periodic paralysis, normokalemic periodic paralysis, Parkinson's disease, malignant hyperthemia, multidrug resistance, myasthenia gravis, myotonic dystrophy, catatonia, tardive dyskinesia, dystonias, peripheral neuropathy, cerebral, neoplasms, prostate cancer, cardiac disorders associated with transport, e.g., angina, bradyarrythmia, tachyarrythmia, hypertension, Long QT syndrome, myocarditis, cardiomyopathy, nemaline myopathy, centronuclear myopathy, lipid myopathy, mitochondrial myopathy, thyrotoxic myopathy, ethanol myopathy, dermatomyositis, inclusion body myositis, infectious myositis, polymyositis, neurological disorders associated with transport, e.g., Alzheimer's disease, amnesia, bipolar disorder, dementia, depression, epilepsy, Tourette's disorder, paranoid psychoses, and schizophrenia, and other disorders associated with transport, e.g., neurofibromatosis, postherpetic neuralgia, trigeminal neuropathy, sarcoidosis, sickle cell anemia, Wilson's disease, cataracts, infertility, pulmonary artery stenosis, sensorineural autosomal deafness, hyperglycemia, hypoglycemia, Grave's disease, goiter, Cushing's disease, Addison's disease, glucose-galactose malabsorption syndrome, hypercholesterolemia, adrenoleukodystrophy, Zellweger syndrome, Menkes disease, occipital horn syndrome, von Gierke disease, cystinuria, iminoglycinuria, Hartup disease, and Fanconi disease, and cancers including adenocarcinoma, leukemia, lymphoma, melanoma, myeloma, sarcoma, teratocarcinoma, and, in particular, cancers of the adrenal gland, bladder, bone, bone marrow, brain, breast; cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; and a viral infection such as those caused by adenoviruses (acute respiratory disease, pneumonia), arenaviruses (lymphocytic choriomeningitis), bunyaviruses (Hantavirus), coronaviruses (pneumonia, chronic bronchitis), hepadnaviruses (hepatitis), herpesviruses (herpes simplex virus, varicefla-zoster virus, Epstein-Barr virus, cytomegalovirus), flaviviruses (yellow fever), orthomyxoviruses (influenza), papillomaviruses (cancer), paramyxoviruses (measles, mumps), picornoviruses (rhinovirus, poliovirus, coxsackie-virus), polyomaviruses (BK virus, JC virus), poxviruses (smallpox), reovirus (Colorado tick fever), retroviruses (human immunodeficiency virus, human T lymphotropic virus), rhabdoviruses (rabies), rotaviruses (gastroenteritis), and togaviruses (encephalitis, rubella). Polynucleotides encoding REMAP may be used in Southern or northern analysis, dot blot, or other membrane-based technologies; in PCR technologies; in dipstick, pin, and multiformat ELISA-like assays; and in microarrays utilizing fluids or tissues from patients to detect altered REMAP expression. Such qualitative or quantitative methods are well known in the art.

In a particular aspect, polynucleotides encoding REMAP may be used in assays that detect the presence of associated disorders, particularly those mentioned above. Polynucleotides complementary to sequences encoding REMAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding REMAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of REMAP, a normal or standard profile for expression is established. This may be accomplished by combining body fluids or cell extracts taken from normal subjects, either animal or human, with a sequence, or a fragment thereof, encoding REMAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding REMAP may involve the use of PCR. These oligomers may be chemically synthesized, generated enzymatically, or produced in vitro. Oligomers will preferably contain a fragment of a polynucleotide encoding REMAP, or a fragment of a polynucleotide complementary to the polynucleotide encoding REMAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding REMAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding REMAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of REMAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, REMAP, fragments of REMAP, or antibodies specific for REMAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for REMAP to quantify the levels of REMAP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding REMAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding REMAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, REMAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between REMAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with REMAP, or fragments thereof, and washed. Bound REMAP is then detected by methods well known in the art. Purified REMAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding REMAP specifically compete with a test compound for binding REMAP. In this manner, antibodies can be used to detect the presence of any peptide which shares one or more antigenic determinants with REMAP.

In additional embodiments, the nucleotide sequences which encode REMAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/306,020, U.S. Ser. No. 60/308,179, U.S. Ser. No. 60/309,702, U.S. Ser. No. 60/311,476, U.S. Ser. No. 60/311,551, U.S. Ser. No. 60/311,718, U.S. Ser. No.60/314,798, U.S. Ser. No. 60/316,0639, and U.S. U.S. Ser. No. 60/317,996, are hereby expressly incorporated by reference.

### EXAMPLES

### I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DHSa, DH10B, or ElectroMAX DH10B from Invitrogen.

### II. Isolation of cDNA Clones

Plasmids obtained as described in Example I were recovered from host cells by in vivo excision using the UNAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

### III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The fill length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:24-46. Fragments from about 20 to about 4000 nucleotides which are useful in hybridization and amplification technologies are described in Table 4, column 2.

### IV. Identification and Editing of Coding Sequences from Genomic DNA

Putative receptors and membrane-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode receptors and membrane-associated proteins, the encoded polypeptides were analyzed by querying against PFAM models for receptors and membrane-associated proteins. Potential receptors and membrane-associated proteins were also identified by homology to Incyte cDNA sequences that had been annotated as receptors and membrane-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

### V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

### VI. Chromosomal Mapping of REMAP Encoding Polynucleotides

The sequences which were used to assemble SEQ ID NO:24-46 were compared with sequences from the Incyte LIFESEQ database and public domain databases using BLAST and other implementations of the Smith-Waterman algorithm. Sequences from these databases that matched SEQ ID NO:24-46 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

### VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7; Ausubel et al., supra, ch. 4).

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding REMAP are analyzed with respect to the tissue sources from which they were derived. For example, some full length sequences are assembled, at east in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding REMAP. cDNA sequences and cDNA library/tissue information are found in the LIFESEQ GOLD database (Incyte Genomics, Palo Alto Calif.).

### VIII. Extension of REMAP Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C. 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., min; Step 5; steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

### IX. Identification of Single Nucleotide Polymorphisms in REMAP Encoding Polynucleotides

Common DNA sequence variants known as single nucleotide polymorphisms-(SNPs) were identified in SEQ ID NO:24-46 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

### X. Labeling and Use of Individual Hybridization Probes

Hybridization probes derived from SEQ ID NO:24-46 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu If-(DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

### XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization-at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

Expression

SEQ ID NO:35 showed differential expression in association with lung cancer, as determined by microarray analysis. Gene expression profiles were obtained by comparing the results of competitive hybridization experiments. Messenger RNA isolated from grossly uninvolved lung tissue with no visible abnormalities was compared to lung squamous cell adenocarcinoma tissue from matched donors (Roy Castle International Centre for Lung Cancer Research, Liverpool, UK). In matched tissue experiments, the expression of SEQ ID NO:35 was increased by at least two-fold in tumorous lung tissue as compared to normal lung tissue from the same donor. Thus, in various embodiments, SEQ ID NO:35 can be used for one or more of the following: i) monitoring treatment of lung cancer, ii) diagnostic assays for lung cancer, and iii) developing therapeutics and/or other treatments for lung cancer.

### XII. Complementary Polynucleotides

Sequences complementary to the REMAP-encoding sequences, or any parts thereof, are used to detect, decrease, or inhibit expression of naturally occurring REMAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of REMAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the REMAP-encoding transcript.

### XIII. Expression of REMAP

Expression and purification of REMAP is achieved using bacterial or virus-based expression systems. For expression of REMAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express REMAP upon induction with isopropyl beta-D-thiogalactopyranoside (IPTG). Expression of REMAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding REMAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, REMAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from REMAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified REMAP obtained by these methods can be used directly in the assays shown in Examples XVII, XVIII, and XIX where applicable.

### XIV. Functional Assays

REMAP function is assessed by expressing the sequences encoding REMAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (i994; Flow Cytometry, Oxford, New York N.Y.).

The influence of REMAP on gene expression can be assessed using highly purified populations of cells transfected with sequences encoding REMAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding REMAP and other genes of interest can be analyzed by northern analysis or microarray techniques.

### XV. Production of REMAP Specific Antibodies

REMAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the REMAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 43 1A peptide synthesizer (Applied Biosystems) using PMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-REMAP activity by, for example, binding the peptide or REMAP to a substrate, blocking with 1% BSA, reacting with rabbit antisera, washing, and reacting with radio-iodinated goat anti-rabbit IgG.

### XVI. Purification of Naturally Occurring REMAP Using Specific Antibodies

Naturally occurring or recombinant REMAP is substantially purified by immunoaffinity chromatography using antibodies specific for REMAP. An immunoaffnity column is constructed by covalently coupling anti-REMAP antibody to an activated chromatographic resin, such as CNBr-activated SEPHAROSE (Amersham Biosciences). After the coupling, the resin is blocked and washed according to the manufacturer's instructions.

Media containing REMAP are passed over the immunoaffinity column, and the column is washed under conditions that allow the preferential absorbance of REMAP (e.g., high ionic strength buffers in the presence of detergent). The column is eluted under conditions that disrupt antibody/REMAP binding (e.g., a buffer of pH 2 to pH 3, or a high concentration of a chaotrope, such as urea or thiocyanate ion), and REMAP is collected.

### XVII. Identification of Molecules which Interact with REMAP

REMAP, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled REMAP, washed, and any wells with labeled REMAP complex are assayed. Data obtained using different concentrations of REMAP are used to calculate values for the number, affinity, and association of REMAP with the candidate molecules.

Alternatively, molecules interacting with REMAP are analyzed using the yeast two-hybrid system as described in Fields, S. and O. Song (1989; Nature 340:245-246), or using commercially available kits based on the two-hybrid system, such as the MATCHMAKER system (Clontech).

REMAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

### XVIII. Demonstration of REMAP Activity

An assay for REMAP activity measures the expression of REMAP on the cell surface. cDNA encoding REMAP is transfected into an appropriate mammalian cell line. Cell surface proteins are labeled with biotin as described (de la Fuente, M. A. et al. (1997) Blood 90:2398-2405). Immunoprecipitations are performed using REMAP-specific antibodies, and immunoprecipitated samples are analyzed using sodium dodecyl sulfate polyacrylamide gel electrophoresis (SDS-PAGE) and immunoblotting techniques. The ratio of labeled immunoprecipitant to unlabeled immunoprecipitant is proportional to the amount of REMAP expressed on the cell surface.

In the alternative, an assay for REMAP activity is based on a prototypical assay for ligand/receptor-mediated modulation of cell proliferation. This assay measures the rate of DNA synthesis in Swiss mouse 3T3 cells. A plasmid containing polynucleotides encoding REMAP is added to quiescent 3T3 cultured cells using transfection methods well known in the art. The transiently transfected cells are then incubated in the presence of [³H]thymidine, a radioactive DNA precursor molecule. Varying amounts of REMAP ligand are then added to the cultured cells. Incorporation of [³H]thymidine into acid-precipitable DNA is measured over an appropriate time interval using a radioisotope counter, and the amount incorporated is directly proportional to the amount of newly synthesized DNA. A linear dose-response curve over at least a hundred-fold REMAP ligand concentration range is indicative of receptor activity. One unit of activity per milliliter is defined as the concentration of REMAP producing a 50% response level, where 100% represents maximal incorporation of [³H]thymidine into acid-precipitable DNA (McKay, I. and I. Leigh, eds. (1993) Growth Factors: A Practical Approach, Oxford University Press, New York N.Y., p. 73.)

In a further alternative, the assay for REMAP activity is based upon the ability of GPCR family proteins to modulate G protein-activated second messenger signal transduction pathways (e.g., cAMP; Gaudin, P. et al. (1998) J. Biol. Chem. 273:4990-4996). A plasmid encoding full length REMAP is transfected into a mammalian cell line (e.g., Chinese hamster ovary (CHO) or human embryonic kidney (HEK-293) cell lines) using methods well-known in the art. Transfected cells are grown in 12-well trays in culture medium for 48 hours, then the culture medium is discarded, and the attached cells are gently washed with PBS. The cells are then incubated in culture medium with or without ligand for 30 minutes, then the medium is removed and cells lysed by treatment with 1 M perchloric acid. The cAMP levels in the lysate are measured by radioimmunoassay using methods well-known in the art. Changes in the levels of cAMP in the lysate from cells exposed to ligand compared to those without ligand are proportional to the amount of REMAP present in the transfected cells.

To measure changes in inositol phosphate levels, the cells are grown in 24-well plates containing 1×10⁵ cells/well and incubated with inositol-free media and [³H]myoinositol, 2 μCi/well, for 48 hr. The culture medium is removed, and the cells washed with buffer containing 10 mM LiCl followed by addition of ligand. The reaction is stopped by addition of perchloric acid. Inositol phosphates are extracted and separated on Dowex AG1-X8 (Bio-Rad) anion exchange resin, and the total labeled inositol phosphates counted by liquid scintillation. Changes in the levels of labeled inositol phosphate from cells exposed to ligand compared to those without ligand are proportional to the amount of REMAP present in the transfected cells.

In a further alternative, the ion conductance capacity of REMAP is demonstrated using an electrophysiological assay. REMAP is expressed by transforming a mammalian cell line such as COS7, HeLa or CHO with a eukaryotic expression vector encoding REMAP. Eukaryotic expression vectors are commercially available, and the techniques to introduce them into cells are well known to those skilled in the art. A small amount of a second plasmid, which expresses any one of a number of marker genes such as β-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of REMAP and β-galactosidase. Transformed cells expressing β-galactosidase are stained blue when a suitable colorimetric substrate is added to the culture media under conditions that are well known in the art. Stained cells are tested for differences in membrane conductance due to various ions by electrophysiological techniques that are well known in the art. Untransformed cells, and/or cells transformed with either vector sequences alone or β-galactosidase sequences alone, are used as controls and tested in parallel. The contribution of REMAP to cation or anion conductance can be shown by incubating the cells using antibodies specific for either REMAP. The respective antibodies will bind to the extracellular side of REMAP, thereby blocking the pore in the ion channel, and the associated conductance.

In a further alternative, REMAP transport activity is assayed by measuring uptake of labeled substrates into Xenopus laevis oocytes. Oocytes at stages V and VI are injected with REMAP mRNA (10 ng per oocyte) and incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of REMAP protein. Oocytes are then transferred to standard uptake medium (100 mM NaCl, 2 mM KCl, 1 mM CaCl₂, 1 mM MgCl₂, 10 mM Hepes/Tris pH 7.5). Uptake of various substrates (e.g., amino acids, sugars, drugs, and neurotransmitters) is initiated by adding a ³H substrate to the oocytes. After incubating for 30 minutes, uptake is terminated by washing the oocytes three times in Na⁺-free medium, measuring the incorporated ³H, and comparing with controls. REMAP activity is proportional to the level of internalized ³H substrate.

In a further alternative, REMAP protein kinase (PK) activity is measured by phosphorylation of a protein substrate using gamma-labeled [³²P]-ATP and quantitation of the incorporated radioactivity using a gamma radioisotope counter. REMAP is incubated with the protein substrate, [³²P]-ATP, and an appropriate kinase buffer. The ³²P incorporated into the product is separated from free [³²P]-ATP by electrophoresis and the incorporated ³²P is counted. The amount of ³²P recovered is proportional to the PK activity of REMAP in the assay. A determination of the specific amino acid residue phosphorylated is made by phosphoamino acid analysis of the hydrolyzed protein.

Transcriptional regulatory activity of REMAP is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (LexAₒₚ) fused to sequences encoding the E. coli LacZ enzyme. The methods for constructing and expressing fusion genes, introducing them into cells, and measuring LacZ enzyme activity, are well known to those skilled in the art. Sequences encoding REMAP are cloned into a plasmid that directs the synthesis of a fusion protein, LexA-REMAP, consisting of REMAP and a DNA-binding domain derived from the LexA transcription factor. The resulting plasmid, encoding a LexA-REMAP fusion protein, is introduced into yeast cells along with a plasmid containing the LexAₒₚ-LacZ reporter gene. The amount of LacZ enzyme activity associated with LexA-NuREC transfected cells, relative to control cells, is proportional to the amount of transcription stimulated by the REMAP.

Phorbol ester binding activity of REMAP is measured using an assay based on the fluorescent phorbol ester sapinotoxin-D (SAPD). Binding of SAPD to REMAP is quantified by measuring the resonance energy transfer from REMAP tryptophans to the 2-(N-methylamino)benzoyl fluorophore of the phorbol ester, as described by Slater et al. ((1996) J. Biol. Chem. 271:4627-4631). Transport activity of REMAP is assayed by measuring uptake of labeled substrates into Xenopus laevis oocytes. Oocytes at stages V and VI are injected with REMAP mRNA (10 ng per oocyte) and incubated for 3 days at 18° C. in OR2 medium (82.5 mM NaCl, 2.5 mM KCl, 1 mM CaCl₂, 1 mM MgCl₂, 1 mM Na₂HPO₄, 5 mM Hepes, 3.8 mM NaOH, 50 μg/ml gentamycin, pH 7.8) to allow expression of REMAP. Oocytes are then transferred to standard uptake medium (100 mM NaCl, 2 mM KCl, 1 mM CaC₂, 1 mM MgCl₂, 10 mM Hepes/Tris pH 7.5). Uptake of various substrates (e.g., amino acids, sugars, drugs, ions, and neurotransmitters) is initiated by adding labeled substrate (e.g. radiolabeled with ³H, fluorescently labeled with rhodamine, etc.) to the oocytes. After incubating for 30 minutes, uptake is terminated by washing the oocytes three times in Na⁺-free medium, measuring the incorporated label, and comparing with controls. REMAP activity is proportional to the level of internalized labeled substrate.

ATPase activity associated with REMAP can be measured by hydrolysis of radiolabeled ATP-[γ-³²P], separation of the hydrolysis products by chromatographic methods, and quantitation of the recovered ³²P using a scintillation counter. The reaction mixture contains ATP-[γ-³²P] and varying amounts of REMAP in a suitable buffer incubated at 37° C. for a suitable period of time. The reaction is terminated by acid precipitation with trichloroacetic acid and then neutralized with base, and an aliquot of the reaction mixture is subjected to membrane or filter paper-based chromatography to separate the reaction products. The amount of ³²P liberated is counted in a scintillation counter. The amount of radioactivity recovered is proportional to the ATPase activity of REMAP in the assay.

Ion channel activity of REMAP is demonstrated using an electrophysiological assay for ion conductance. REMAP can be expressed by transforming a mammalian cell line such as COS7, HeLa or CHO with a eukaryotic expression vector encoding REMAP. Eukaryotic expression vectors are commercially available, and the techniques to introduce them into cells are well known to those skilled in the art. A second plasmid which expresses any one of a number of marker genes, such as β-galactosidase, is co-transformed into the cells to allow rapid identification of those cells which have taken up and expressed the foreign DNA. The cells are incubated for 48-72 hours after transformation under conditions appropriate for the cell line to allow expression and accumulation of REMAP and β-galactosidase.

Transformed cells expressing β-galactosidase are stained blue when a suitable colorimetric substrate is added to the culture media under conditions that are well known in the art. Stained cells are tested for differences in membrane conductance by electrophysiological techniques that are well known in the art. Untransformed cells, and/or cells transformed with either vector sequences alone or β-galactosidase sequences alone, are used as controls and tested in parallel. Cells expressing REMAP will have higher anion or cation conductance relative to control cells. The contribution of REMAP to conductance can be confirmed by incubating the cells using antibodies specific for REMAP. The antibodies will bind to the extracellular side of REMAP, thereby blocking the pore in the ion channel, and the associated conductance.

Alternatively, ion channel activity of REMAP is measured as current flow across a REMAP-containing Xenopus laevis oocyte membrane using the two-electrode voltage-clamp technique (Ishi et al., supra; Jegla, T. and L. Salkoff (1997) J. Neurosci. 17:32-44). REMAP is subcloned into an appropriate Xenopus oocyte expression vector, such as pBF, and 0.5-5 ng of mRNA is injected into mature stage IV oocytes. Injected oocytes are incubated at 18° C. for 1-5 days. Inside-out macropatches are excised into an intracellular solution containing 116 mM K-gluconate, 4 mM KCl, and 10 mM Hepes (pH 7.2). The intracellular solution is supplemented with varying concentrations of the REMAP mediator, such as cAMP, cGMP, or Ca⁺² (in the form of CaCl₂), where appropriate. Electrode resistance is set at 2-5 MΩ and electrodes are filled with the intracellular solution lacking mediator. Experiments are performed at room temperature from a holding potential of 0 mV. Voltage ramps (2.5 s) from −100 to 100 mV are acquired at a sampling frequency of 500 Hz. Current measured is proportional to the activity of REMAP in the assay.

### XIX. Identification of REMAP Ligands

REMAP is expressed in a eukaryotic cell line such as CHO (Chinese Hamster Ovary) or HEK (Human Embryonic Kidney) 293 which have a good history of GPCR expression and which contain a wide range of G-proteins allowing for functional coupling of the expressed REMAP to downstream effectors. The transformed cells are assayed for activation of the expressed receptors in the presence of candidate ligands. Activity is measured by changes in intracellular second messengers, such as cyclic AMP or Ca²⁺. These may be measured directly using standard methods well known in the art, or by the use of reporter gene assays in which a luminescent protein (e.g. firefly luciferase or green fluorescent protein) is under the transcriptional control of a promoter responsive to the stimulation of protein kinase C by the activated receptor (Milligan, G. et al. (1996) Trends Pharmacol. Sci. 17:235-237). Assay technologies are available for both of these second messenger systems to allow high throughput readout in multi-well plate format, such as the adenylyl cyclase activation FlashPlate Assay (NEN Life Sciences Products), or fluorescent Ca²⁺ indicators such as Fluo-4 AM (Molecular Probes) in combination with the FLIPR fluorimetric plate reading system (Molecular Devices). In a more generic version of this assay, changes in membrane potential caused by ionic flux across the plasma membrane are measured using oxonyl dyes such as DiBAC₄ (Molecular Probes). DiBAC₄ equilibrates between the extracellular solution and cellular sites according to the cellular membrane potential. The dye's fluorescence intensity is 20-fold greater when bound to hydrophobic intracellular sites, allowing detection of DiBAC₄ entry into the cell (Gonzalez, J. E. and P. A. Negulescu (1998) Curr. Opin. Biotechnol. 9:624-631). Candidate agonists or antagonists may be selected from known ion channel agonists or antagonists, peptide libraries, or combinatorial chemical libraries. In cases where the physiologically relevant second messenger pathway is not known, REMAP may be coexpressed with the G-proteins G_{α15/16} which have been demonstrated to couple to a wide range of G-proteins (Offermanns, S. and M. I. Simon (1995) J. Biol. Chem. 270:15175-15180), in order to funnel the signal transduction of the REMAP through a pathway involving phospholipase C and Ca²⁺ mobilization. Alternatively, REMAP may be expressed in engineered yeast systems which lack endogenous GPCRs, thus providing the advantage of a null background for REMAP activation screening. These yeast systems substitute a human GPCR and G_{α} protein for the corresponding components of the endogenous yeast pheromone receptor pathway. Downstream signaling pathways are also modified so that the normal yeast response to the signal is converted to positive growth on selective media or to reporter gene expression (Broach, J. R. and J. Thomer (1996) Nature 384 (supp.):14-16). The receptors are screened against putative ligands including known GPCR ligands and other naturally occurring bioactive molecules. Biological extracts from tissues, biological fluids and cell supernatants are also screened.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | Incyte | | Incyte | |
|---|---|---|---|---|---|
| Incyte | Polypeptide | Polypeptide | Polynucleotide | Polynucleotide | Incyte Full |
| Project ID | SEQ ID NO: | ID | SEQ ID NO: | ID | Length Clones |
| 5771933 | 1 | 5771933CD1 | 24 | 5771933CB1 | 90215359CA2 |
| 70475510 | 2 | 70475510CD1 | 25 | 70475510CB1 | |
| 566361 | 3 | 566361CD1 | 26 | 566361CB1 | |
| 71969340 | 4 | 71969340CD1 | 27 | 71969340CB1 | |
| 6772808 | 5 | 6772808CD1 | 28 | 6772808CB1 | |
| 60137669 | 6 | 60137669CD1 | 29 | 60137669CB1 | 90110422CA2 |
| 1987928 | 7 | 1987928CD1 | 30 | 1987928CB1 | 90110123CA2, |
| | | | | | 90110131CA2, |
| | | | | | 90110139CA2, |
| | | | | | 90110147CA2 |
| 7268131 | 8 | 7268131CD1 | 31 | 7268131CB1 | 90108068CA2 |
| 7285339 | 9 | 7285339CD1 | 32 | 7285339CB1 | |
| 7495197 | 10 | 7495197CD1 | 33 | 7495197CB1 | |
| 3954126 | 11 | 3954126CD1 | 34 | 3954126CB1 | |
| 7499693 | 12 | 7499693CD1 | 35 | 7499693CB1 | |
| 2187465 | 13 | 2187465CD1 | 36 | 2187465CB1 | |
| 3718011 | 14 | 3718011CD1 | 37 | 3718011CB1 | |
| 7500509 | 15 | 7500509CD1 | 38 | 7500509CB1 | 90175928CA2 |
| 7497865 | 16 | 7497865CD1 | 39 | 7497865CB1 | 90197602CA2 |
| 3116578 | 17 | 3116578CD1 | 40 | 3116578CB1 | |
| 2797803 | 18 | 2797803CD1 | 41 | 2797803CB1 | |
| 5433453 | 19 | 5433453CD1 | 42 | 5433453CB1 | 2600495CA2, |
| | | | | | 3533193CA2 |
| 6246071 | 20 | 6246071CD1 | 43 | 6246071CB1 | 6246071CA2 |
| 7500557 | 21 | 7500557CD1 | 44 | 7500557CB1 | |
| 6978182 | 22 | 6978182CD1 | 45 | 6978182CB1 | 90111161CA2 |
| 1985321 | 23 | 1985321CD1 | 46 | 1985321CB1 | |

TABLE 2

| | Incyte | GenBank ID NO: | | |
|---|---|---|---|---|
| Polypeptide | Polypeptide | or PROTEOME | Probability | |
| SEQ ID NO: | ID | ID NO: | Score | Annotation |
| 1 | 5771933CD1 | g4335933 | 1.0E−70 | [Gallus gallus] ChT1 |
| | | | | Chretien, I., et al. (1998) Eur. J. Immunol. 28: 4094-4104 |
| 2 | 70475510CD1 | g17864081 | 0.0 | [f1][Mus musculus] PPAR gamma coactivator-1beta protein |
| | | | | Kakuma, T., et al. (2000) Endocrinology 141: 4576-4582 |
| 3 | 566361CD1 | g178252 | 5.0E−38 | [Homo sapiens] epidermal growth factor receptor-related protein |
| | | | | Kielman, M. F. et al. (1993) Homology of a 130-kb region enclosing the alpha- |
| | | | | globin gene cluster, the alpha-locus controlling region, and two non-globin genes |
| | | | | in human and mouse. Mamm. Genome 4: 314-323. |
| 4 | 71969340CD1 | g4049585 | 2.0E−18 | [f1][Homo sapiens] Slit-1 protein |
| | | | | Itoh, A. et al. (1998) Cloning and expressions of three mammalian homologues of |
| | | | | Drosophila slit suggest possible roles for Slit in the formation and maintenance of |
| | | | | the nervous system. |
| | | | | Brain Res. Mol. Brain Res. 62: 175-186. |
| 5 | 6772808CD1 | g7715916 | 0.0 | [Mus musculus] SorCSb splice variant of the VPS10 domain receptor SorCS |
| | | | | Hermey, G. and Schaller, H. C. (2000) Biochim. Biophys. Acta 1491: 350-354 |
| | | | | Alternative splicing of murine SorCS leads to two forms of the receptor that differ |
| | | | | completely in their cytoplasmic tails |
| 6 | 60137669CD1 | g311817 | 2.2E−28 | [Mus musculus] erythroid ankyrin |
| | | | | Birkenmeier, C. S. et al. (1993) J. Biol. Chem. 268 (13), 9533-9540 |
| 7 | 1987928CD1 | g13649390 | 1.2E−28 | [Homo sapiens] MS4A8B protein |
| | | | | Liang, Y. et al. (2001) Genomics 72 (2), 119-127 |
| 8 | 7268131CD1 | g7861753 | 2.2E−13 | [Mus musculus] GABA-A receptor epsilon-like subunit |
| | | | | Sinkkonen, S. T. et al. (2000) GABA(A) receptor epsilon and theta subunits |
| | | | | display unusual structural variation between species and are enriched in the rat |
| | | | | locus ceruleus. J. Neurosci. 20: 3588-3595. |
| 9 | 7285339CD1 | g7861753 | 5.1E−14 | [Mus musculus] GABA-A receptor epsilon-like subunit |
| | | | | Sinkkonen, S.T. et al. (2000) GABA(A) receptor epsilon and theta subunits |
| | | | | display unusual structural variation between species and are enriched in the rat |
| | | | | locus ceruleus. J. Neurosci. 20: 3588-3595. |
| 10 | 7495197CD1 | g20269724 | 0.0 | [f1][Mus musculus] neuropilin and tolloid like-1 |
| | | | | Stohr, H. et al. A novel gene encoding a putative transmembrane protein with two |
| | | | | extracellular CUB domains and a low-density lipoprotein class A module: |
| | | | | isolation of alternatively spliced isoforms in retina and brain. Gene 286 (2), 223- |
| | | | | 231 (2002). |
| | | g2367641 | 2.9E−23 | [Rattus norvegicus] neuropilin-2 |
| | | | | Kolodkin, A. L. (1997) Neuropilin is a semaphorin III receptor. Cell 90: 753-762. |
| 11 | 3954126CD1 | g1763306 | 0.0 | [Rattus norvegicus] Munc13-3 |
| 12 | 7499693CD1 | g20269724 | 5.0E−163 | [f1][Mus musculus] neuropilin and tolloid like-1 |
| | | | | Stohr, H. et al. A novel gene encoding a putative transmembrane protein with two |
| | | | | extracellular CUB domains and a low-density lipoprotein class A module: |
| | | | | isolation of alternatively spliced isoforms in retina and brain. Gene 286 (2), 223- |
| | | | | 231 (2002). |
| | | g11907926 | 4.5E−25 | [Homo sapiens] neuropilin-2b(O) |
| | | | | Rossignol, M. et al. Genomic organization of human neuropilin-1 and neuropilin-2 |
| | | | | genes: identification and distribution of splice variants and soluble isoforms. |
| | | | | Genomics 70 (2), 211-222 (2000). |
| 13 | 2187465CD1 | g5453324 | 3.1E−112 | [Mus musculus] syntaxin4-interacting protein synip |
| | | | | Min, J. et al. (1999) Synip: a novel insulin-regulated syntaxin 4-binding protein |
| | | | | mediating GLUT4 translocation in adipocytes. Mol. Cell 3: 751-760. |
| 15 | 7500509CD1 | g298665 | 4.4E−168 | [Homo sapiens] CD68 = 110 kda transmembrane glycoprotein [human, |
| | | | | promonocyte cell line U937, Peptide, 354 aa] |
| | | | | Holness, C. L. and Simmons, D. L. (1993) Molecular cloning of CD68, a human |
| | | | | macrophage marker related to lysosomal glycoproteins. Blood. 81: 1607-1613. |
| 16 | 7497865CD1 | g339762 | 2.3E−235 | [Homo sapiens] tumor necrosis factor receptor 2 related protein |
| | | | | Baens, M. et al. (1993) Construction and evaluation of a hncDNA library of |
| | | | | human 12p transcribed sequences derived from a somatic cell hybrid. Genomics. |
| | | | | 16: 214-218. |
| | | g600223 | 1.0E−159 | [f1][Mus musculus] lymphotoxin-beta receptor |
| | | | | Nakamura, T. et al. The murine lymphotoxin-beta receptor cDNA: isolation by the |
| | | | | signal sequence trap and chromosomal mapping. Genomics 30 (2), 312-319 |
| | | | | (1995). |
| 22 | 6978182CD1 | g9858571 | 7.0E−45 | [f1][Homo sapiens] coxsackie virus and adenovirus receptor |

TABLE 3

| | | Amino | | | | |
|---|---|---|---|---|---|---|
| SEQ | Incyte | Acid | Potential | Potential | | Analytical |
| ID | Polypeptide | Resi- | Phosphorylation | Glycosylation | Signature Sequences, | Methods |
| NO: | ID | dues | Sites | Sites | Domains and Motifs | and Databases |
| 1 | 5771933CD1 | 423 | S256 S265 S342 | N32 N38 N134 | Signal cleavage: M1-V21 | SPSCAN |
| | | | S392 S414 T25 | N169 N236 N255 | | |
| | | | T238 T308 T333 | | | |
| | | | T346 T350 T390 | | | |
| | | | | | Signal Peptide: M1-A16 | HMMER |
| | | | | | Signal Peptide: M1-S20 | |
| | | | | | Signal Peptide: M1-V21 | |
| | | | | | Signal Peptide: M1-V24 | |
| | | | | | Non-cytosolic domain: M1-V269 | TMHMMER |
| | | | | | Transmembrane region: G270-F292 | |
| | | | | | Cytosolic domain: A293-A423 | |
| | | | | | Immunoglobulin domain: G190-A249, | HMMER_PFAM |
| | | | | | G36-V154 | |
| | | | | | CELL SURFACE A33 | BLAST_PRODOM |
| | | | | | ANTIGEN PRECURSOR | |
| | | | | | IMMUNOGLOBULIN | |
| | | | | | FOLD LIPOPROTEIN | |
| | | | | | PALMITATE | |
| | | | | | GLYCOPROTEIN PD155626: | |
| | | | | | G162-E330 | |
| 2 | 70475510CD1 | 972 | S18 S33 S38 S56 | N857 | PPAR GAMMA COACTIVATOR 1 | BLAST_PRODOM |
| | | | S64 S75 S142 S146 | | PD145040: G19-S132, C502-G718, | |
| | | | S161 S188 S212 | | S305-P360, Q158-P227, | |
| | | | S229 S285 S338 | | D506-D518, S348-E396 | |
| | | | S339 S348 S357 | | | |
| | | | S428 S473 S479 | | | |
| | | | S496 S519 S528 | | | |
| | | | S592 S637 S731 | | | |
| | | | S747 S830 S835 | | | |
| | | | S863 S941 S950 | | | |
| | | | S953 T87 T319 | | | |
| | | | T440 T475 T564 | | | |
| | | | T722 T739 T779 | | | |
| | | | T817 T896 T937 | | | |
| | | | | | ATP/GTP-binding site motif A | MOTIFS |
| | | | | | (P-loop): A946-S953 | |
| 3 | 566361CD1 | 827 | S16 S21 S61 S73 | N26 N350 N555 | Rhomboid family: P619-Y761 | HMMER_PFAM |
| | | | S88 S119 S148 | N722 | | |
| | | | S195 S210 S227 | | | |
| | | | S247 S266 S272 | | | |
| | | | S352 S370 S419 | | | |
| | | | S433 S516 S767 | | | |
| | | | T482 T526 T582 | | | |
| | | | T813 Y422 | | | |
| | | | | | Cytosolic domains: 1-374, | TMHMMER |
| | | | | | 648-658, 714-719, 763-774 | |
| | | | | | Transmembrane domains: 375-397, | |
| | | | | | 625-647, 659-681, 691- | |
| | | | | | 713, 720-739, 743-762, | |
| | | | | | 775-797 | |
| | | | | | Non-cytosolic domains: 398-624, | |
| | | | | | 682-690, 740-742, 798-827 | |
| 4 | 71969340CD1 | 828 | S151 S183 S267 | N59 N85 N90 | Signal Peptides: M1-A21, | HMMER |
| | | | S461 S524 S551 | N122 N210 N349 | M1-A25, M1-A27 | |
| | | | S592 S645 S648 | N376 N391 | | |
| | | | S735 S764 S775 | | | |
| | | | S783 T61 T92 | | | |
| | | | T311 T465 T517 | | | |
| | | | T769 Y471 Y750 | | | |
| | | | | | Signal Peotides: M1-A21, | HMMER |
| | | | | | M1-A25, M1-A27 | |
| | | | | | Leucine Rich Repeat: N85-F108, | HMMER_PFAM |
| | | | | | N157-A180, K133-P156, | |
| | | | | | T61-G84, N109-G132 | |
| | | | | | Leucine rich repeat C-terminal | HMMER_PFAM |
| | | | | | domain: N190-G235 | |
| | | | | | Non-cytosolic domain: 1-417 | TMHMMER |
| | | | | | Transmembrane domain: 418-440 | |
| | | | | | Cytosolic domain: 441-828 | |
| 5 | 6772808CD1 | 1168 | S105 S111 S127 | N184 N352 N433 | Signal_cleavage: M1-G33 | SPSCAN |
| | | | S201 S258 S298 | N765 N776 N816 | | |
| | | | S325 S393 S417 | N847 N908 N929 | | |
| | | | S457 S562 S613 | | | |
| | | | S653 S667 S685 | | | |
| | | | S703 S849 S850 | | | |
| | | | S942 S978 S1008 | | | |
| | | | S1049 S1142 S1161 | | | |
| | | | T52 T215 T238 | | | |
| | | | T247 T347 T577 | | | |
| | | | T724 T786 T901 | | | |
| | | | T1030 T1050 | | | |
| | | | T1156 Y536 Y678 | | | |
| | | | | | Signal Peptide: M1-G33, M1-G34, | HMMER |
| | | | | | Q11-G33, Q11-G34, A12-G33 | |
| | | | | | Non-cytosolic domain: M1-T1097 | TMHMMER |
| | | | | | Transmembrane domain: H1098-Y1120 | |
| | | | | | Cytosolic domain: K1121-I1168 | |
| | | | | | BNR repeat: F569-Q580, W208-K219, | HMMER_PFAM |
| | | | | | L256-K267, F492-L503, W611-K622 | |
| | | | | | PKD (polycystic kidney disease | HMMER_PFAM |
| | | | | | protein)domain: K795-T887 | |
| | | | | | GLYCOPROTEIN PROTEIN | BLAST_PRODOM |
| | | | | | PRECURSOR SIGNAL | |
| | | | | | TRANSMEMBRANE LR11 | |
| | | | | | PUTATIVE MEMBRANE | |
| | | | | | VACUOLAR RECEPTOR | |
| | | | | | PD007682: W658-K795 | |
| | | | | | YIL173W; MEMBRANE; DM02204 | BLAST_DOMO |
| | | | | | P40438\|562-714: V663-E812 | |
| | | | | | S50354\|562-714: V663-E812 | |
| | | | | | P40890\|562-714: V663-E812 | |
| | | | | | P53751\|123-281: V663-E812 | |
| | | | | | Cell attachment sequence: | MOTIFS |
| | | | | | R512-D514 | |
| 6 | 60137669CD1 | 300 | S172 S241 T6 T52 | N246 | Ank repeat: T212-E244, C143-S176, | HMMER_PFAM |
| | | | T188 Y139 | | A42-K74, I109-N142, D9-K41, | |
| | | | | | K245-I276, L177-T210, D75-T105 | |
| 7 | 1987928CD1 | 240 | T51 T164 T180 | N18 N130 | Cytosolic Domain: R96-G101, | TMHMMER |
| | | | Y172 | | M159-R170 | |
| | | | | | Transmembrane Domain: V73-V95, | |
| | | | | | I102-S124, S139-L158, G171-F193 | |
| | | | | | Non-cytosolic Domain: M1-K72, | |
| | | | | | V125-S138, G194-V240 | |
| | | | | | RECEPTOR HIGH AFFINITY | BLAST_PRODOM |
| | | | | | IMMUNOGLOBULIN EPSILON | |
| | | | | | BETASUBUNIT FCERI IGE FC | |
| | | | | | IGEBINDING PD023556: E43-D160 | |
| | | | | | ANTIGEN CD20 SURFACE BCELL | BLAST_PRODOM |
| | | | | | TRANSMEMBRANE PHOSPHORYLATION | |
| | | | | | BLYMPHOCYTE B1 LEU16 BP35 | |
| | | | | | PD039784: P62-D160 | |
| | | | | | B-CELL SURFACE ANTIGEN CD20 | BLAST_DOMO |
| | | | | | DM08044\|P11836\|1-296: | |
| | | | | | P62-D160 | |
| | | | | | DM08044\|P19437\|1-290: | |
| | | | | | P62-D160 | |
| | | | | | BETA; IMMUNOGLOBULIN; EPSILON; | BLAST_DOMO |
| | | | | | AFFINITY; | |
| | | | | | DM03973\|P20490\|1-234: | |
| | | | | | P30-N165 | |
| | | | | | DM03973\|Q01362\|1-243: | |
| | | | | | L29-D160 | |
| | | | | | Immunoglobulins and major | MOTIFS |
| | | | | | histocompatibility complex | |
| | | | | | proteins signature: F193-H199 | |
| 8 | 7268131CD1 | 394 | S4, S17, S28, S100, | N53 | | |
| | | | S110, S124, S174, | | | |
| | | | S205, S238, T151, | | | |
| | | | T162, T262, T344 | | | |
| 9 | 7285339CD1 | 340 | S4, S17, S28, S100, | N53 | | |
| | | | S110, S124, S174, | | | |
| | | | S205, S238, T151, | | | |
| | | | T162, T262 | | | |
| 10 | 7495197CD1 | 525 | S121, S141, S233, | N298, N332, | Signal cleavage: M1-A14 | SPSCAN |
| | | | S234, S278, S325, | N438, N473, | | |
| | | | S369, S416, S431, | N521 | | |
| | | | S440, S494, S498, | | | |
| | | | S514, T15, T19, | | | |
| | | | T23, T27, T187, | | | |
| | | | T324, T389, T522 | | | |
| | | | | | CUB domain: C33-Y144, C164-F276 | HMMER-PFAM |
| | | | | | CUB domain proteins profile: | BLIMPS-BLOCKS |
| | | | | | BL01180: C88-G98, G107-S120 | |
| | | | | | (p = 0.0012) | |
| | | | | | LDL-receptor class A: BL01209: | BLIMPS-BLOCKS |
| | | | | | C303-E319 | |
| | | | | | Low-density lipoprotein receptor | HMMER-PFAM |
| | | | | | domain: P282-E320 | |
| | | | | | GLYCOPROTEIN DOMAIN EGF-LIKE | BLAST-PRODOM |
| | | | | | PROTEIN PRECURSOR SIGNAL | |
| | | | | | RECEPTOR INTRINSIC FACTOR B12 | |
| | | | | | REPEAT: PD000165: C33-Y144 | |
| | | | | | C1R/C1S REPEAT: | BLAST-DOMO |
| | | | | | DM00162\|I49540\|748-862: | |
| | | | | | G43-N145; | |
| | | | | | DM00162\|P98063\|755-862: | |
| | | | | | G43-N145; | |
| | | | | | DM00162\|I49540\|438-552: | |
| | | | | | C33-Y144; | |
| | | | | | DM00162\|P98063\|438-549: | |
| | | | | | C33-Y144 | |
| 11 | 3954126CD1 | 2214 | S52 S76 S93 S111 | N74 N325 N493 | C2 domain: I1222-I1313, | HMMER_PFAM |
| | | | S121 S126 S130 | N497 N503 N574 | V2063-V2153 | |
| | | | S136 S157 S167 | N813 N842 N874 | | |
| | | | S196 S254 S273 | N891 N939 | | |
| | | | S279 S286 S298 | N1277 N1741 | | |
| | | | S320 S394 S435 | N1873 N2115 | | |
| | | | S448 S452 S469 | N2174 | | |
| | | | S483 S488 S498 | | | |
| | | | S502 S505 S537 | | | |
| | | | S547 S549 S559 | | | |
| | | | S580 S582 S600 | | | |
| | | | S649 S671 S682 | | | |
| | | | S762 S788 S806 | | | |
| | | | S820 S894 S971 | | Phorbol esters/diacylglycerol | HMMER_PFAM |
| | | | S997 S998 S1007 | | binding domain (C1 domain): | |
| | | | S1034 S1155 S1196 | | H1098-C1147 | |
| | | | S1210 S1219 S1305 | | | |
| | | | S1429 S1464 S1466 | | | |
| | | | S1489 S1504 S1514 | | | |
| | | | S1572 S1732 S1786 | | | |
| | | | S1876 S1891 S1903 | | | |
| | | | S2009 S2038 S2111 | | Phorbol esters/diacylglycerol | BLIMPS_BLOCKS |
| | | | S2136 S2176 S2189 | | binding domain proteins | |
| | | | S2209 T23 T29 | | BL00479: H1098-G1120, | |
| | | | T58 T62 T77 T109 | | Q1124-C1139 | |
| | | | T202 T217 T302 | | | |
| | | | T479 T543 T596 | | | |
| | | | T617 T715 T840 | | | |
| | | | T846 T896 T912 | | | |
| | | | T916 T941 T1043 | | | |
| | | | T1215 T1256 | | | |
| | | | T1279 T1312 | | | |
| | | | T1333 T1506 | | | |
| | | | T1553 T1585 | | | |
| | | | T1601 T1845 | | | |
| | | | T1971 T1984 | | | |
| | | | T2064 T2192 Y308 | | | |
| | | | Y867 Y1419 Y1554 | | | |
| | | | | | Phorbol esters/diacylglycerol | PROFILESCAN |
| | | | | | binding domain: Y1110-R1174 | |
| | | | | | C2 domain signature and | PROFILESCAN |
| | | | | | profile: S1196-T1258 | |
| | | | | | C2 domain signature PR00360: | BLIMPS_PRINTS |
| | | | | | K1237-V1249, G1261-E1274, | |
| | | | | | I1282-D1290 | |
| | | | | | PHORBOL ESTER BINDING | BLAST_PRODOM |
| | | | | | PROTEIN UNC13 | |
| | | | | | MUNC13 MUNC132 MUNC131 | |
| | | | | | MUNC133 PD010159: | |
| | | | | | T1312-T1940, P1934-L2073, | |
| | | | | | K2040-K2062, N745-L819, | |
| | | | | | H780-V811, N754-S820 | |
| | | | | | MUNC133 PHORBOL ESTER | BLAST_PRODOM |
| | | | | | BINDING PD141195: N493-T916 | |
| | | | | | PHORBOL ESTER BINDING | BLAST_PRODOM |
| | | | | | MUNC132 MUNC133 | |
| | | | | | PD042959: N110-T406 | |
| | | | | | PHORBOL ESTER BINDING UNC13 | BLAST_PRODOM |
| | | | | | PROTEIN MUNC13 MUNC131 MUNC133 | |
| | | | | | MUNC132 PHORBOL ESTER/ | |
| | | | | | DIACYLGLYCEROL-BINDING | |
| | | | | | PD016836: P917-P1097 | |
| | | | | | MUNC13 | BLAST_DOMO |
| | | | | | DM08803\|I61776\|1013-1154: | |
| | | | | | K1257-D1399 | |
| | | | | | DM08803\|A57607\|726-867: | |
| | | | | | K1257-D1399 | |
| | | | | | C2-DOMAIN | BLAST_DOMO |
| | | | | | DM00150\|P27715\|801-928: | |
| | | | | | K1205-K1331 | |
| | | | | | DM00150\|I61776\|1811-1943: | |
| | | | | | D2041-L2171 | |
| | | | | | C2 domain signature: A1229-Y1244 | MOTIFS |
| | | | | | Phorbol esters/diacylglycerol | MOTIFS |
| | | | | | binding domain: H1098-C1147 | |
| 12 | 7499693CD1 | 487 | S142 S143 S182 | N347 N415 N437 | Signal_cleavage: M1-A26, | SPSCAN |
| | | | S191 S246 S291 | | M1-G33 | |
| | | | S364 S391 S408 | | | |
| | | | S417 S444 T87 | | | |
| | | | T133 T210 T214 | | | |
| | | | T439 T446 | | | |
| | | | | | Signal Peptide: M1-G22, | HMMER |
| | | | | | M1-A26, M1-A24 | |
| | | | | | Extracellular domain: M1-K307 | TMHMMER |
| | | | | | Transmembrane domain: T308-V330 | |
| | | | | | Intracellular domain: Q331-F487 | |
| | | | | | CUB domain: C45-Y156, C177-F289 | HMMER_PFAM |
| | | | | | GLYCOPROTEIN DOMAIN EGF-LIKE | BLAST_PRODOM |
| | | | | | PROTEIN PRECURSOR SIGNAL | |
| | | | | | RECEPTOR INTRINSIC FACTOR | |
| | | | | | B12 REPEAT PD000165: T51-Y156 | |
| | | | | | C1R/C1S REPEAT | BLAST_DOMO |
| | | | | | DM00162\|I49540\|748-862: | |
| | | | | | T51-S157 | |
| | | | | | DM00162\|P98063\|755-862: | |
| | | | | | T51-S157 | |
| | | | | | DM00162\|I49540\|438-552: | |
| | | | | | C45-Y156 | |
| | | | | | DM00162\|P98063\|438-549: | |
| | | | | | C45-Y156 | |
| 13 | 2187465CD1 | 405 | S12 S82 S99 S122 | N4 N117 N172 | PDZ domain (Also known as | HMMER_PFAM |
| | | | S142 S163 S189 | N183 | DHR or GLGF): Q21-E102 | |
| | | | S212 S252 S292 | | | |
| | | | T154 T157 T313 | | | |
| | | | | | Cytosolic domain: M1-S381 | TMHMMER |
| | | | | | Transmembrane domain: S382-L404 | |
| | | | | | Non-cytosolic domain: N405-N405 | |
| | | | | | PDZ DOMAIN PROTEINS | BLIMPS_PFAM |
| | | | | | (ALS PF00595: L64-N74 | |
| | | | | | PROTEIN SH3 DOMAIN REPEAT | BLIMPS_PRODOM |
| | | | | | PD00289: G67-G80 | |
| | | | | | PROTEIN DOMAIN PROTEASE | BLAST_PRODOM |
| | | | | | PHOSPHATASE SH3 REPEAT PDZ | |
| | | | | | TYROSINE PRECURSOR HYDROLASE | |
| | | | | | PD000073: I23-A93 | |
| | | | | | GLGF DOMAIN | BLAST_DOMO |
| | | | | | DM00224\|P55196\|980-1073: | |
| | | | | | L14-R92 | |
| 14 | 3718011CD1 | 910 | S5 S41 S79 S115 | N153 N226 N329 | Cytosolic domains: M1-K294 | TMHMMER |
| | | | S169 S256 S366 | N361 N493 N777 | L393-S457 E528-M554 | |
| | | | S367 S485 S640 | N790 N802 | N694-D720 V848-E910 | |
| | | | S642 S847 S860 | | Transmembrane domains: | |
| | | | T83 T88 T135 | | I295-V317 L370-F392 A458-V480 | |
| | | | T435 T525 T535 | | Q505-Y527 F555-F572 | |
| | | | T542 T544 T551 | | I671-V693 I721-I743 I825-S847 | |
| | | | T646 T805 T874 | | Non-cytosolic domains: | |
| | | | Y405 Y813 | | A318-K369 F481-P504 K573-M670 | |
| | | | | | A744-N824 | |
| | | | | | PROTEIN AAC3RFC5 INTERGENIC | BLAST_PRODOM |
| | | | | | REGION TRANSMEMBRANE F56A8.1 | |
| | | | | | PD025564: F373-S747, M741-D766 | |
| | | | | | Growth factor and cytokines | MOTIFS |
| | | | | | receptors family signature 1: | |
| | | | | | C319-W332 | |
| 15 | 7500509CD1 | 327 | S23 S29 S236 S267 | N61 N69 N91 | signal_cleavage: M1-A16 | SPSCAN |
| | | | S289 S322 T26 T34 | N99 N137 N172 | | |
| | | | T125 T129 | N219 N234 N252 | | |
| | | | | | Signal Peptide: M1-S18, M1-G20, | HMMER |
| | | | | | M1-T21, M1-T22, M1-823, | |
| | | | | | M1-R25 | |
| | | | | | Lysosome-associated membrane | HMMER_PFAM |
| | | | | | glycoprotein (Lamp): M1-L327 | |
| | | | | | Cytosolic domain: R318-L327 | TMHMMER |
| | | | | | Transmembrane domain: L295-I317 | |
| | | | | | Non-cytosolic domain: M1-L294 | |
| | | | | | Lysosome-associated membrane | BLMPS_BLOCKS |
| | | | | | glycoproteins duplicated | |
| | | | | | domain proteins BL00310: | |
| | | | | | T38-T73, L240-S286, E128-M154, | |
| | | | | | F230-S254, D264-R318 | |
| | | | | | Lysosome-associated membrane- | BLIMPS_PRINTS |
| | | | | | glycoprotein signature | |
| | | | | | PR00336: G131-Y155, A242-I256, | |
| | | | | | G279-R291, S292-F314, F314-A326 | |
| | | | | | PRECURSOR TRANSMEMBRANE | BLAST_PRODOM |
| | | | | | GLYCOPROTEIN SIGNAL | |
| | | | | | LYSOSOME MEMBRANE LYSOSOME- | |
| | | | | | ASSOCIATED LAMP-2 ANTIGEN | |
| | | | | | LYSOSOMAL ALTERNATIVE SPLICING | |
| | | | | | PD005775: S29-L327 | |
| | | | | | PROTEIN PRECURSOR GLYCOPROTEIN | BLAST_PRODOM |
| | | | | | SIGNAL REPEAT ANTIGEN | |
| | | | | | SURFACE MEROZOITE CELL | |
| | | | | | TRANSMEMBRANE PD000546: S18-G131 | |
| | | | | | LAMP GLYCOPROTEINS TRANSMEMBRANE | BLAST_DOMO |
| | | | | | AND CYTOPLASMIC DOMAIN DM01644 | |
| | | | | | \|P34810\|36-353: L15-L327 | |
| | | | | | \|P31996\|27-325: T38-L327 | |
| | | | | | \|P05300\|71-413: H59-L327 | |
| | | | | | \|A60534\|76-405: A85-Q325 | |
| | | | | | LAMP glycoproteins | MOTIFS |
| | | | | | transmembrane and cytoplasmic | |
| | | | | | domain signature: C287-Q325 | |
| 16 | 7497865CD1 | 416 | S50 S68 S99 S163 | N21 N158 | TNFR/NGFR cysteine-rich | HMMER_PFAM |
| | | | S304 S404 T23 T63 | | region: C24-C61, C151-C191, | |
| | | | T98 T103 T121 | | C107-L137, C64-C105 | |
| | | | T133 T170 T190 | | | |
| | | | Y31 | | | |
| | | | | | Cytosolic domain: K230-D416 | TMHMMER |
| | | | | | Transmembrane domain: L207-W229 | |
| | | | | | Non-cytosolic domain: M1-M206 | |
| | | | | | TNFR/NGFR family cysteine-rich | BLIMPS_BLOCKS |
| | | | | | region proteins BL00652: | |
| | | | | | C39-V49, C97-C107 | |
| | | | | | Diacylglycerol kinase ca | BLMPS_PFAM |
| | | | | | PF00781: H147-K152, P194-F225, | |
| | | | | | I278-Q301, T382-L393 | |
| | | | | | LYMPHOTOXIN BETA RECEPTOR | BLAST_PRODOM |
| | | | | | PRECURSOR TRANSMEMBRANE | |
| | | | | | GLYCOPROTEIN REPEAT SIGNAL | |
| | | | | | TUMOR NECROSIS FACTOR | |
| | | | | | PD037872: R106-G400 | |
| | | | | | PD028432: G5-T63 | |
| | | | | | LYMPHOTOXIN-BETA RECEPTOR | BLAST_DOMO |
| | | | | | CHAIN DM06944 | |
| | | | | | \|P36941\|204-434: A185-D416 | |
| | | | | | \|P50284\|206-414: S187-G400 | |
| | | | | | TNFR/NGFR FAMILY CYSTEINE-RICH | BLAST_DOMO |
| | | | | | REGION DM00218 | |
| | | | | | \|P36941\|119-202: K100-T184 | |
| | | | | | \|P36941\|39-117: E20-S99 | |
| | | | | | TNFR/NGFR family cysteine-rich | MOTIFS |
| | | | | | region signature: C24-C61, C64-C105 | |
| 17 | 3116578CD1 | 635 | S29 S90 S188 S201 | N66 N114 N134 | signal_cleavage: M1-S19 | SPSCAN |
| | | | S217 S376 S382 | N433 N602 | | |
| | | | S525 S604 T116 | | | |
| | | | T205 T230 T245 | | | |
| | | | T276 Y135 | | | |
| | | | | | Signal Peptide: M1-S19, M1-A20, | HMMER |
| | | | | | M1-A21, M1-A24, M1-P25, | |
| | | | | | M1-S28, M1-G30, M1-D32 | |
| | | | | | Cytosolic domains: M1-R6, | TMHMMER |
| | | | | | L189-R247, Q302-K313, P371-S389, | |
| | | | | | K497-D502, V560-G565, R628-I635 | |
| | | | | | Transmembrane domains: A7-S29, | |
| | | | | | V166-S188, G248-F267, F282-L301, | |
| | | | | | I314-Y333, V348-V370, W390-V412, | |
| | | | | | L474-Y496, I503-T522, L537-P559, | |
| | | | | | L566-V588, H608-Y627 | |
| | | | | | Non-cytosolic domains: | |
| | | | | | G30-P165, K268-V281, C334-G347, | |
| | | | | | P413-I473, K523-N536, F589-E607 | |
| 18 | 2797803CD1 | 478 | S42 S134 S204 | N456 | SAM domain (Sterile alpha motif): | HMMER_PFAM |
| | | | S331 S438 S449 | | R73-Q139 | |
| | | | T76 T109 T111 | | | |
| | | | T325 T355 T379 | | | |
| | | | T419 Y212 Y246 | | | |
| | | | | | Cytosolic domains: M1-K214, | TMHMMER |
| | | | | | L283-R294, S362-R381, N431-G478 | |
| | | | | | Transmembrane domains: T215-H237, | |
| | | | | | I260-L282, L295-V317, A339-F361, | |
| | | | | | S382-A404, Y408-A430 | |
| | | | | | Non-cytosolic domains: E238-R259, | |
| | | | | | P318-R338, H405-H407 | |
| | | | | | Leucine zipper pattern: L284-L305 | MOTIFS |
| 19 | 5433453CD1 | 634 | S124 S162 S177 | | Cytosolic domains: M1-R189, | TMHMMER |
| | | | S289 S452 S551 | | G250-Y343 | |
| | | | T30 T570 T631 | | Transmembrane domains: Y190-A212, | |
| | | | | | G227-A249, T344-I366 | |
| | | | | | Non-cytosolic domains: P213-A226, | |
| | | | | | D367-D634 | |
| | | | | | Iron dependant repressor PF01325: | BLIMPS_PFAM |
| | | | | | E157-E169 | |
| | | | | | Leucine zipper pattern: L311-L332 | MOTIFS |
| | | | | | Cell attachment sequence: R461-D463 | MOTIFS |
| 20 | 6246071CD1 | 152 | | | Cytosolic domains: M1-R60, | TMHMMER |
| | | | | | T121-T121 | |
| | | | | | Transmembrane domains: L61-T83, | |
| | | | | | A98-F120, A122-P144 | |
| | | | | | Non-cytosolic domains: T84-A97, | |
| | | | | | G145-Q152 | |
| | | | | | Eukaryotic thiol (cysteine) | MOTIFS |
| | | | | | proteases histidine active site: | |
| | | | | | L77-H87 | |
| 21 | 7500557CD1 | 308 | S42 S134 S204 T76 | | SAM domain (Sterile alpha | HMMER_PFAM |
| | | | T109 T111 Y212 | | motif): R73-Q139 | |
| | | | Y246 | | | |
| | | | | | Cytosolic domains: M1-K214, | TMHMMER |
| | | | | | H285-V308 | |
| | | | | | Transmembrane domains: T215-H237, | |
| | | | | | W262-L284 | |
| | | | | | Non-cytosolic domain: E238-P261 | |
| 22 | 6978182CD1 | 431 | S3 S166 S295 S304 | N102 N108 N204 | signal_cleavage: M1-A21 | SPSCAN |
| | | | S393 T184 T201 | N308 N360 N389 | | |
| | | | | | Signal Peptide: M1-A21, Q4-A21, | HMMER |
| | | | | | M1-S22, M1-L23, M1-E24, | |
| | | | | | M1-S26, M1-S28, M1-P29 | |
| | | | | | Immunoglobulin domain: G37-V122, | HMMER_PFAM |
| | | | | | G158-A217 | |
| | | | | | Cytosolic domain: R269-V431 | TMHMMER |
| | | | | | Transmembrane domain: A246-W268 | |
| | | | | | Non-cytosolic domain: M1-G245 | |
| | | | | | Myelin P0 protein signature | BLIMPS_PRINTS |
| | | | | | PR00213: A85-L112, D114-P143 | |
| | | | | | CELL SURFACE A33 ANTIGEN | BLAST_PRODOM |
| | | | | | PRECURSOR IMMUNOGLOBULIN | |
| | | | | | FOLD LIPOPROTEIN PALMITATE | |
| | | | | | GLYCOPROTEIN PD155626: G130-P291 | |
| | | | | | PRECURSOR GLYCOPROTEIN SIGNAL | BLAST_PRODOM |
| | | | | | CHANNEL TRANSMEMBRANE | |
| | | | | | IMMUNOGLOBULIN FOLD PROTEIN | |
| | | | | | MYELIN SODIUM PD013099: I32-S145 | |
| 23 | 1985321CD1 | 93 | T17 T33 Y25 | | Signal_cleavage: M1-A50 | SPSCAN |
| | | | | | Non-cytosolic domain: M1-R23 | TMHMMER |
| | | | | | Transmembrane domain: G24-F46 | |
| | | | | | Cytosolic domain: G47-V93 | |
| | | | | | Immunoglobulins and major | MOTIFS |
| | | | | | histocompati-bility complex | |
| | | | | | proteins signature: F46-H52 | |

TABLE 4

| Polynucleotide | |
|---|---|
| SEQ ID NO:/ | |
| Incyte ID/Sequence | |
| Length | Sequence Fragments |
| 24/5771933CB1/ | 1-601, 1-1442, 325-592, 335-536, 494-1193, 494-1253, 494-1254, 494-1260, 494-1341, 494-1391, 494-1416, 496- |
| 1748 | 1315, 498-1422, 500-1371, 520-1393, 592-1388, 624-1389, 646-1397, 691-795, 697-1442, 752-809, 756-1393, 770- |
| | 1393, 974-1389, 1099-1606, 1484-1748, 1518-1619, 1666-1748 |
| 25/70475510CB1/ | 1-429, 41-538, 43-127, 50-211, 51-245, 53-275, 79-550, 81-329, 84-538, 84-554, 87-1000, 89-127, 125-752, 210- |
| 4028 | 847, 210-878, 359-769, 371-889, 430-770, 518-1131, 531-1103, 535-1138, 571-1134, 583-1211, 609-1243, 615- |
| | 1196, 742-1320, 748-1195, 806-1422, 851-1065, 931-1583, 946-1623, 1092-1643, 1104-1722, 1132-1706, 1212- |
| | 1492, 1237-1483, 1243-1717, 1251-1724, 1252-1808, 1281-1556, 1296-1529, 1327-1793, 1333-1925, 1421-2001, |
| | 1455-1971, 1573-1846, 1573-1945, 1573-2136, 1592-2165, 1606-2210, 1607-2140, 1607-2247, 1608-2107, 1609- |
| | 2184, 1612-2049, 1634-2156, 1651-1794, 1655-2049, 1664-2232, 1743-2359, 1783-2424, 1792-1951, 1793-2071, |
| | 1800-2387, 1803-2359, 1805-2445, 1808-2285, 1830-2482, 1846-2423, 1902-2230, 1922-2393, 1929-2067, 1930- |
| | 2499, 1953-2584, 1962-2079, 1966-2459, 1968-2326, 1968-2345, 1970-2597, 1987-2559, 2000-2571, 2013-2598, |
| | 2014-2617, 2021-2508, 2021-2531, 2035-2687, 2042-2603, 2042-2604, 2049-2686, 2065-2701, 2074-2557, 2076- |
| | 2666, 2108-2548, 2155-2662, 2156-2718, 2169-2805, 2185-2749, 2203-2751, 2209-2757, 2253-2799, 2254-2652, |
| | 2260-2678, 2294-2816, 2316-2736, 2328-2805, 2330-2945, 2355-3037, 2364-2714, 2389-2907, 2440-2631, |
| | 2456-2835, 2571-3079, 2614-3065, 2637-2887, 2637-3202, 2662-3079, 2671-2919, 2699-2899, 2705-3333, 2818- |
| | 3298, 2934-3385, 2935-3081, 2990-3392, 3047-3241, 3049-3223, 3082-4028 |
| 26/566361CB1/ | 1-260, 1-444, 1-553, 2-260, 8-607, 159-688, 161-688, 237-732, 271-732, 339-611, 395-875, 659-1198, 686-1129, |
| 3320 | 714-1460, 744-1353, 828-1098, 852-1414, 1081-1678, 1083-1245, 1156-1622, 1230-1719, 1285-1568, 1354-1636, |
| | 1354-1718, 1409-1660, 1449-1690, 1451-1753, 1551-1787, 1760-2320, 1865-2321, 1986-2279, 1991-2648, 2022- |
| | 2253, 2037-2474, 2105-2367, 2105-2565, 2137-2322, 2137-2542, 2169-2496, 2191-2743, 2201-2674, 2209-2723, |
| | 2253-2879, 2294-2320, 2299-2880, 2308-2356, 2343-2890, 2487-2796, 2581-2847, 2581-3132, 2598-2688, 2615- |
| | 3141, 2622-3209, 2626-2874, 2635-2858, 2639-3311, 2666-3169, 2728-3043, 2744-3252, 2744-3320, 2749-3000, |
| | 2749-3274 |
| 27/71969340CB1/ | 1-772, 1-2609, 100-760, 125-774, 207-694, 211-474, 211-480, 211-657, 211-688, 211-735, 211-742, 211-775, 211- |
| 2914 | 815, 211-850, 211-993, 215-784, 215-882, 215-923, 216-798, 235-756, 285-689, 357-1024, 381-1024, 383-1024, |
| | 430-1024, 485-1131, 488-754, 488-1014, 526-1024, 529-1213, 584-1252, 589-1024, 604-1197, 607-1208, 631-1291, |
| | 690-1367, 714-1172, 739-958, 753-1148, 767-1281, 772-994, 831-1171, 831-1172, 887-1331, 890-1173, 919-1132, |
| | 965-1628, 1003-1588, 1025-1268, 1072-1693, 1108-1422, 1112-1361, 1113-1364, 1120-1748, 1134-1715, 1152- |
| | 1794, 1191-1461, 1239-1399, 1262-1579, 1267-1504, 1286-1518, 1317-1583, 1350-1586, 1380-1524, 1387-1795, |
| | 1502-2155, 1505-1707, 1514-2150, 1561-1962, 1606-1905, 1692-2161, 1707-1874, 1718-2115, 1749-2143, 1757- |
| | 1993, 1759-2120, 1812-2609, 2196-2860, 2229-2431, 2231-2431, 2320-2914, 2625-2886 |
| 28/6772808CB1/ | 1-614, 1-619, 1-621, 152-622, 550-688, 550-992, 550-1172, 550-1189, 550-1264, 642-781, 878-1267, 878-3660, |
| 3990 | 1622-1859, 1622-1939, 1622-2216, 1668-2259, 1776-2259, 1898-2259, 2046-2259, 2209-2342, 2284-2699, 2553- |
| | 3083, 2553-3108, 2553-3113, 2553-3114, 2556-3114, 2579-3114, 2586-3114, 3523-3990 |
| 29/60137669CB1/ | 1-269, 1-709, 119-385, 175-606, 210-430, 242-808, 268-863, 309-891, 328-791, 329-909, 337-909, 349-1034, 393- |
| 1198 | 793, 403-893, 434-909, 573-1153, 609-1159, 620-870, 643-1106, 643-1133, 644-1198, 666-923, 671-1140, 688-864, |
| | 693-1159, 696-762, 702-933, 702-1129, 702-1133, 703-1140, 704-802, 704-1158, 705-1144, 713-1159, 745-1140, |
| | 757-1140, 759-1140, 774-1147, 796-1035, 862-1140 |
| 30/1987928CB1/ | 1-535, 24-235, 166-700, 329-701, 384-700, 459-1123, 472-1098, 497-1205, 541-1198, 555-1297, 569-1271, 592- |
| 1297 | 856, 603-1188, 621-876, 621-1290, 651-1271 |
| 31/7268131CB1/ | 1-471, 1-549, 1-599, 5-597, 6-547, 6-653, 9-562, 14-515, 20-434, 20-512, 22-618, 24-731, 27-555, 30-601, 32-610, |
| 2482 | 40-587, 51-876, 64-429, 68-422, 77-693, 100-391, 104-607, 104-782, 105-619, 105-697, 106-631, 107-693, 135- |
| | 578, 135-622, 149-876, 154-585, 160-747, 171-437, 173-876, 183-424, 187-876, 190-876, 207-642, 217-659, 259- |
| | 876, 264-758, 303-748, 304-876, 313-605, 321-876, 323-876, 332-876, 348-876, 384-876, 392-1003, 397-1153, 400- |
| | 1096, 445-876, 447-722, 464-1014, 466-876, 471-876, 494-1080, 563-814, 571-1100, 602-867, 659-1136, 726-1074, |
| | 776-1081, 801-1212, 801-1347, 845-1212, 871-1137, 871-1481, 875-1515, 888-1145, 935-1212, 1075-1693, 1079- |
| | 1222, 1079-1679, 1142-1281, 1164-1321, 1165-1808, 1165-2027, 1166-1877, 1168-1777, 1181-1815, 1204-1643, |
| | 1225-1906, 1226-1330, 1226-1351, 1226-1538, 1226-1600, 1226-1632, 1226-1643, 1226-1667, 1226-1677, 1226- |
| | 1684, 1226-1687, 1226-1690, 1226-1700, 1226-1710, 1226-1766, 1226-1848, 1226-1866, 1226-1873, 1226-1913, |
| | 1226-1943, 1226-2013, 1226-2095, 1226-2154, 1229-1963, 1266-1477, 1266-1787, 1281-1787, 1300-1765, |
| | 1305-1932, 1312-1949, 1316-1915, 1324-1588, 1364-2127, 1383-2170, 1387-1639, 1410-1887, 1439-1960, 1450- |
| | 2055, 1463-2174, 1464-2424, 1501-2106, 1519-1856, 1524-2152, 1534-2109, 1556-2353, 1558-2353, 1572-2010, |
| | 1572-2013, 1572-2117, 1572-2147, 1573-2261, 1573-2415, 1616-1898, 1621-1860, 1638-2256, 1640-2371, 1641- |
| | 1961, 1656-2128, 1657-1898, 1665-1896, 1669-1757, 1676-2350, 1680-2179, 1756-2384, 1777-2459, 1790-2482, |
| | 1791-2407, 1792-2437, 1792-2482, 1798-2386, 1832-2459, 1837-2406, 1848-2476, 1851-2482, 1854-2479, 1859- |
| | 2482, 1864-2386, 1873-2474, 1881-2431, 1882-2453, 1891-2469, 1893-2481, 1893-2482, 1894-2443, 1895-2460, |
| | 1896-2451, 1900-2422, 1900-2460, 1912-2480, 1913-2453, 1936-2450, 1938-2478, 1947-2479, 1968-2479, 1973- |
| | 2482, 1977-2482, 1983-2407, 1998-2482, 2014-2482, 2016-2482, 2025-2480, 2063-2482, 2067-2458, 2068-2459, |
| | 2079-2482, 2104-2457, 2108-2446, 2108-2481, 2109-2395, 2113-2459, 2133-2407, 2176-2459, 2178-2482, 2195- |
| | 2459, 2203-2459, 2228-2453, 2384-2480, 2386-2481 |
| 32/7285339CB1/ | 1-554, 1-604, 19-520, 25-517, 69-434, 105-396, 110-702, 137-583, 165-752, 269-763, 318-610, 499-1085, 607-872, |
| 2323 | 781-1086, 806-1216, 850-1216, 851-1446, 876-1142, 903-1187, 904-1800, 940-1216, 1062-1333, 1115-1406, 1224- |
| | 1494, 1230-1482, 1230-1722, 1269-1577, 1271-1752, 1273-1537, 1282-1803, 1293-1898, 1355-1414, 1358-2014, |
| | 1377-1952, 1442-2279, 1484-1804, 1500-1741, 1508-1739, 1519-2193, 1675-2302, 1725-2296, 1736-2323, 1737- |
| | 2286, 1738-2303, 1739-2294, 1743-2303, 1755-2323, 1947-2300, 2227-2323, 2229-2323 |
| 33/7495197CB1/ | 1-278, 1-291, 1-292, 209-652, 211-651, 497-700, 611-854, 618-1324, 618-1335, 618-1336, 618-1337, 618-1363, 618- |
| 2232 | 1377, 618-1410, 618-1411, 618-1527, 618-1545, 618-1577, 618-1595, 628-1174, 659-1279, 693-913, 705-1116, 807- |
| | 1784, 823-1784, 829-1778, 831-1784, 839-1407, 857-1780, 891-1784, 970-1784, 975-1786, 976-1784, 978-1784, |
| | 983-1224, 983-1494, 983-1724, 1003-1784, 1019-1784, 1051-1195, 1111-1723, 1163-1762, 1166-1446, 1166-1682, |
| | 1166-1717, 1166-1722, 1168-1784, 1208-1792, 1220-1792, 1241-1768, 1263-1882, 1308-1802, 1334-1780, 1340- |
| | 1626, 1340-1882, 1407-1978, 1409-2102, 1440-1981, 1446-1904, 1542-1798, 1557-1755, 1576-2213, 1598-2232, |
| | 1601-1939, 1725-2225, 1736-2231, 1758-2232, 1884-2111, 1987-2231, 1987-2232, 2022-2231, 2022-2232 |
| 34/3954126CB1/ | 1-566, 336-795, 536-3426, 3210-3396, 3210-3427, 3212-3291, 3342-3496, 3342-3733, 3342-3761, 3342-3845, 3342- |
| 7590 | 3846, 3342-3848, 3342-3850, 3342-3926, 3342-3951, 3342-3962, 3342-3970, 3342-3975, 3342-4001, 3342-4015, |
| | 3342-4043, 3342-4259, 3357-4244, 3387-4351, 3452-4348, 3703-4086, 3895-4016, 3895-4071, 3895-4103, 3895- |
| | 4218, 3895-4221, 3895-4292, 3895-4308, 3895-4317, 3895-4321, 3895-4325, 3895-4328, 3895-4382, 3895-4394, |
| | 3895-4407, 3895-4497, 3895-4502, 3895-4522, 3895-4537, 3895-4550, 3895-4563, 3895-4641, 3895-4658, 3895- |
| | 4670, 3895-4686, 3905-4906, 3921-4424, 3946-4504, 3949-4705, 4000-4850, 4190-5177, 4191-5276, 4203-4907, |
| | 4236-4487, 4292-4818, 4294-4903, 4377-5050, 4425-5099, 4437-5259, 4472-5200, 4477-5034, 4483-5085, 4498- |
| | 5274, 4516-5259, 4535-5374, 4550-5146, 4554-5377, 4561-5263, 4564-5259, 4569-5262, 4571-5377, 4587-5377, |
| | 4588-5259, 4612-5377, 4613-5259, 4617-5259, 4636-5377, 4643-5377, 4656-5377, 4674-5377, 4681-5377, 4683- |
| | 5377, 4685-5377, 4694-5377, 4697-5377, 4700-5377, 4706-5377, 4712-5245, 4714-5377, 4743-5259, 4766-5377, |
| | 4833-5376, 4839-5377, 4864-5377, 4867-5377, 4990-5254, 5074-5377, 5177-5743, 5652-6404, 5652-6441, |
| | 5666-6436, 5769-6436, 6352-6762, 6352-6943, 6521-6943, 6530-7046, 6551-6733, 6551-7121, 6836-7100, 6836- |
| | 7428, 6885-7146, 6963-7384, 6969-7322, 7008-7365, 7176-7424, 7320-7590 |
| 35/7499693CB1/ | 1-814, 1-2257, 700-967, 841-1231, 879-1097, 879-1238, 879-1289, 879-1311, 879-1321, 879-1337, 879-1370, 879- |
| 3285i/ | 1374, 879-1376, 879-1392, 879-1396, 879-1406, 879-1411, 879-1413, 879-1418, 879-1438, 879-1439, 879-1442, |
| | 879-1443, 879-1445, 879-1448, 879-1451, 879-1459, 879-1463, 879-1464, 879-1470, 879-1480, 879-1484, 879- |
| | 1486, 879-1489, 879-1498, 879-1547, 879-1673, 887-1554, 893-1416, 908-1519, 909-1474, 910-1518, 913-1414, |
| | 924-1294, 927-1036, 940-1532, 942-1464, 951-1479, 955-1489, 991-1564, 998-1596, 1001-1404, 1007-1649, 1011- |
| | 1516, 1019-1598, 1038-1659, 1050-1686, 1055-1740, 1061-1716, 1073-1707, 1078-1500, 1088-1645, 1092-1703, |
| | 1099-1680, 1106-1617, 1106-1644, 1111-1686, 1113-1643, 1113-1726, 1135-1640, 1135-1731, 1142-1703, 1142- |
| | 1707, 1143-1630, 1143-1760, 1147-1779, 1158-1399, 1158-1402, 1168-1740, 1168-1797, 1169-1835, 1179-1421, |
| | 1180-1596, 1201-1705, 1212-1642, 1225-1852, 1232-1853, 1249-1791, 1249-1889, 1252-1769, 1262-1883, 1269- |
| | 1835, 1289-1421, 1295-1747, 1304-1756, 1314-1855, 1320-1609, 1331-1616, 1337-1595, 1371-1908, 1373-1734, |
| | 1375-1839, 1411-2141, 1484-2064, 1484-2065, 1509-2077, 1567-2104, 1579-2085, 1594-2256, 1604-2184, 1616- |
| | 1911, 1618-2128, 1621-2131, 1629-2250, 1645-2256, 1664-2256, 1683-2258, 1693-2243, 1706-2222, 1712-2248, |
| | 1714-2495, 1733-2009, 1738-2170, 1742-2095, 1748-2495, 1751-2214, 1751-2218, 1759-2298, 1771-2319, 1793- |
| | 2256, 1806-2189, 1807-2209, 1809-2256, 1811-2258, 1813-2256, 1820-2256, 1852-2252, 1856-2255, 1877-2257, |
| | 1892-2495, 1893-2495, 1935-2188, 1954-2593, 1971-2494, 1987-2495, 2007-2298, 2022-2295, 2034-2298, 2042- |
| | 2544, 2075-2506, 2077-2337, 2100-2348, 2114-2257, 2126-2938, 2126-2969, 2129-2415, 2159-2212, 2212-2533, |
| | 2293-2560, 2322-2632, 2355-2996, 2356-2645, 2433-2994, 2522-2855, 2568-2852, 2574-2816, 2574-3068, 2618- |
| | 3285, 2623-2693 |
| 36/2187465CB1/ | 1-230, 1-480, 1-572, 1-591, 1-599, 1-629, 21-141, 21-525, 47-262, 92-695, 95-739, 302-913, 335-963, 336-915, 385- |
| 1825 | 966, 405-963, 473-1107, 510-1181, 511-1059, 545-1183, 547-960, 550-1183, 573-1183, 609-1183, 610-1183, 642- |
| | 1183, 691-1183, 905-1361, 933-1118, 1103-1183, 1184-1430, 1184-1598, 1184-1697, 1184-1704, 1184-1825, 1230- |
| | 1721 |
| 37/3718011CB1/ | 1-212, 2-245, 6-208, 50-120, 156-447, 217-581, 237-850, 245-335, 245-814, 326-523, 326-3126, 460-523, 525-808, |
| 3214 | 525-922, 551-837, 551-1078, 562-1151, 715-1326, 791-1067, 791-1301, 791-1567, 809-1038, 923-1173, 964-1264, |
| | 1007-1466, 1039-1173, 1070-1677, 1082-1566, 1093-1652, 1141-1734, 1148-1675, 1174-1340, 1211-1624, 1220- |
| | 1591, 1280-1483, 1301-1789, 1341-1483, 1341-1561, 1383-1906, 1395-1664, 1395-1935, 1423-1666, 1483-1724, |
| | 1483-2157, 1484-1787, 1503-2066, 1545-1825, 1545-2045, 1927-2554, 1956-2055, 2056-2186, 2066-2556, 2187- |
| | 2589, 2242-2492, 2242-2506, 2290-2861, 2331-2986, 2342-2760, 2350-2556, 2384-3111, 2393-2589, 2393-2701, |
| | 2596-2905, 2680-2961, 2693-2916, 2693-3214, 2702-2905, 2752-2968, 2754-2965, 2881-3097 |
| 38/7500509CB1/ | 1-1477, 19-301, 46-296, 46-588, 48-271, 49-293, 51-327, 51-712, 53-279, 53-312, 58-497, 59-373, 63-301, 63-350, |
| 1597 | 63-395, 64-614, 65-334, 67-315, 70-334, 70-497, 121-533, 121-700, 122-775, 125-356, 126-383, 133-372, 139-413, |
| | 147-841, 161-709, 165-670, 170-988, 171-449, 184-393, 184-421, 191-454, 191-674, 191-796, 199-231, 199-244, |
| | 199-256, 199-266, 199-280, 199-290, 199-293, 199-297, 203-578, 206-297, 207-798, 210-297, 212-297, 216-519, |
| | 219-297, 222-297, 238-297, 241-487, 243-552, 245-479, 249-793, 250-297, 251-489, 251-495, 252-297, 260-297, |
| | 264-297, 264-300, 270-297, 271-804, 276-563, 276-916, 282-533, 283-525, 283-774, 283-803, 288-536, 289-361, |
| | 289-369, 289-383, 289-386, 289-387, 290-387, 293-387, 295-974, 296-572, 296-816, 297-817, 298-567, 299-985, |
| | 300-387, 300-568, 302-817, 302-922, 304-460, 304-507, 305-557, 305-933, 309-387, 312-1002, 317-1043, 318-458, |
| | 322-547, 331-387, 339-387, 340-886, 340-960, 341-387, 342-540, 347-912, 353-587, 353-635, 353-832, 360-939, |
| | 361-387, 361-568, 369-944, 369-1215, 380-620, 383-788, 387-709, 387-714, 390-551, 392-526, 400-1054, |
| | 401-1079, 407-974, 410-860, 417-1039, 417-1 114, 418-548, 418-987, 422-932, 422-1065, 431-970, 432-853, 432- |
| | 1036, 432-1037, 436-915, 442-678, 442-703, 443-852, 455-743, 456-1117, 462-743, 466-1092, 468-707, 476-975, |
| | 496-1139, 513-765, 513-803, 533-783, 533-791, 536-789, 538-780, 538-1208, 539-659, 540-827, 544-779, 550- |
| | 1057, 550-1114, 555-824, 558-809, 560-816, 560-831, 561-807, 562-884, 565-1193, 565-1354, 566-1116, 574-842, |
| | 574-1186, 575-794, 589-840, 594-1272, 595-1202, 597-887, 600-856, 601-1323, 603-857, 605-872, 606-862, 606- |
| | 865, 606-892, 606-1271, 610-1014, 611-855, 611-901, 612-864, 617-1176, 621-772, 629-1371, 646-1112, 647-1337, |
| | 649-901, 655-1114, 655-1117, 655-1133, 657-1090, 659-842, 659-883, 659-897, 659-1310, 659-1332, 659-1381, |
| | 660-910, 661-928, 662-919, 665-1292, 674-898, 677-920, 677-928, 677-1175, 680-892, 682-1261, 689-904, 689- |
| | 990, 689-1213, 695-946, 703-964, 705-946, 705-997, 706-1133, 706-1253, 707-994, 711-1110, 715-961, 725-934, |
| | 727-953, 738-1298, 745-925, 749-938, 749-1032, 750-1369, 750-1395, 756-1349, 764-1004, 765-1026, 767-1003, |
| | 777-1021, 781-1049, 781-1494, 785-1372, 785-1468, 787-1074, 789-1036, 789-1044, 793-1052, 804-996, |
| | 805-1093, 806-1064, 806-1457, 826-1070, 827-1060, 837-1137, 837-1434, 839-1129, 855-1102, 856-1071, 860- |
| | 1488, 863-1126, 863-1504, 872-1114, 904-1360, 905-1169, 905-1552, 908-1447, 911-1597, 929-1206, 929-1503, |
| | 933-1225, 940-1197, 940-1203, 940-1553, 946-1212, 946-1525, 947-1180, 947-1535, 952-1199, 952-1506, 952- |
| | 1545, 956-1222, 956-1409, 956-1568, 963-1145, 964-1201, 969-1234, 975-1568, 979-1235, 980-1204, 981-1450, |
| | 984-1217, 986-1442, 986-1530, 999-1292, 1007-1545, 1018-1271, 1018-1569, 1022-1278, 1037-1272, 1039-1114, |
| | 1041-1568, 1049-1562, 1059-1307, 1067-1321, 1067-1327, 1083-1336, 1088-1381, 1107-1376, 1120-1373, 1207- |
| | 1227, 1207-1240, 1207-1241, 1348-1378, 1348-1382 |
| 39/7497865CB1/ | 1-529, 1-1883, 50-339, 245-724, 249-724, 323-362, 381-614, 382-672, 411-597, 416-1093, 426-661, 432-1062, 433- |
| 1923 | 835, 442-858, 446-998, 461-737, 461-793, 473-789, 474-1137, 482-789, 483-744, 504-1106, 509-636, 513-660, 535- |
| | 1100, 535-1165, 538-782, 542-1532, 557-1095, 563-1202, 583-828, 589-712, 592-867, 594-871, 599-841, 600-913, |
| | 601-789, 601-861, 601-883, 609-1235, 612-877, 618-1249, 624-1247, 633-766, 636-1238, 643-798, 658-723, 662- |
| | 916, 664-916, 684-789, 704-1243, 711-1293, 720-1237, 721-1162, 726-1227, 740-1517, 747-1472, 748-1432, 774- |
| | 1432, 778-1427, 782-1437, 783-1312, 787-1461, 788-1195, 791-1467, 813-1408, 821-1487, 827-1233, 838-1163, |
| | 844-1156, 844-1395, 850-1571, 855-1585, 856-1372, 857-1184, 863-1672, 888-1393, 894-1477, 897-1183, 904- |
| | 1421, 910-1417, 913-1158, 913-1200, 926-1600, 950-1693, 959-1204, 959-1495, 962-1209, 976-1669, 986-1192, |
| | 988-1383, 988-1464, 994-1248, 1001-1228, 1001-1362, 1001-1508, 1001-1539, 1001-1554, 1001-1565, 1001-1596, |
| | 1001-1610, 1001-1616, 1002-1536, 1002-1678, 1005-1345, 1008-1621, 1010-1227, 1011-1617, 1012-1197, 1019- |
| | 1286, 1022-1736, 1026-1575, 1029-1749, 1030-1310, 1030-1545, 1030-1553, 1039-1607, 1045-1497, |
| | 1045-1524, 1046-1630, 1047-1672, 1049-1290, 1058-1637, 1066-1561, 1066-1654, 1067-1193, 1068-1330, 1068- |
| | 1608, 1070-1721, 1071-1923, 1072-1284, 1072-1713, 1076-1710, 1078-1728, 1079-1403, 1082-1645, 1084-1348, |
| | 1091-1346, 1091-1357, 1104-1656, 1104-1673, 1111-1616, 1116-1372, 1119-1399, 1121-1796, 1128-1384, 1128- |
| | 1573, 1130-1518, 1132-1355, 1140-1423, 1153-1378, 1727-1823 |
| 40/3116578CB1/ | 1-389, 1-418, 28-658, 65-766, 82-808, 83-808, 100-517, 100-555, 100-651, 100-658, 100-690, 101-370, 131-604, |
| 3025 | 131-606, 146-539, 153-697, 169-627, 192-623, 192-625, 192-645, 192-662, 197-809, 200-809, 238-808, 258-1035, |
| | 284-863, 412-975, 417-931, 423-1112, 553-1142, 620-866, 685-900, 763-1278, 808-1342, 899-1496, 958-1268, |
| | 1083-1643, 1152-3025, 1162-1431, 1162-1644, 1162-1702, 1192-1671, 1195-1629, 1236-1868, 1268-1621, 1332- |
| | 1540, 1408-1989, 1464-1970, 1469-1746, 1477-1977, 1485-2077, 1486-1709, 1486-1881, 1516-2019, 1523-2073, |
| | 1589-1882, 1673-2200, 1673-2315, 1689-2291, 1721-2331, 1731-2331, 1761-2121, 1773-1988, 1773-2026, 1776- |
| | 2320, 1790-2329, 1822-2094, 1849-2479, 1913-2155, 1921-2391, 1940-2787, 2136-2912, 2436-3012 |
| 41/2797803CB1/ | 1-864, 126-391, 126-601, 150-402, 173-628, 264-834, 626-1062, 684-1448, 699-862, 803-1484, 943-1238, 954- |
| 1870 | 1636, 961-1518, 1026-1730, 1035-1472, 1126-1395, 1133-1373, 1205-1870 |
| 42/5433453CB1/ | 1-653, 38-580, 71-609, 86-1452, 88-288, 88-502, 120-775, 157-617, 157-620, 157-745, 158-695, 341-722, 428- |
| 2628 | 1010, 491-1208, 773-1415, 1029-1570, 1145-1767, 1301-1703, 1321-1643, 1351-1725, 1381-1887, 1409-1844, 1417- |
| | 2378, 1419-2272, 1484-1786, 1493-1740, 1529-1992, 1561-2061, 1571-1836, 1571-1890, 1686-2628, 1688-2628, |
| | 1890-2620, 1898-2628 |
| 43/6246071CB1/ | 1-523, 13-694, 111-565, 191-568, 214-563, 298-694 |
| 694 | |
| 44/7500557CB1/ | 1-863, 126-391, 126-601, 150-402, 173-628, 174-863, 174-1359, 242-703, 264-702, 264-834, 265-894, 304-722, 308- |
| 1359 | 722, 317-825, 417-787, 450-744, 450-787, 450-820, 450-834, 450-897, 450-970, 451-897, 451-916, 451-969, 471- |
| | 897, 478-742, 479-835, 516-897, 517-896, 517-897, 517-912, 517-916, 517-969, 517-970, 517-979, 518-897, 518- |
| | 970, 521-1027, 532-897, 532-916, 532-970, 553-969, 560-1170, 699-862, 747-1344, 788-897, 788-1170, 917-1354 |
| 45/6978182CB1/ | 1-739, 1-1091, 31-733, 95-742, 134-742, 145-738, 145-742, 145-746, 146-745, 178-746, 442-1013, 550-940, 551- |
| 1585 | 940, 574-940, 638-1039, 646-1118, 969-1584, 969-1585, 970-1585, 971-1504, 974-1585, 978-1585, 994-1584, 995- |
| | 1585, 1091-1252 |
| 46/1985321CB1/ | 1-88, 1-263, 20-556, 33-719, 33-739, 37-271, 37-511, 37-517, 37-528, 37-554, 37-569, 37-575, 37-583, 37-588, 37- |
| 1495 | 612, 37-625, 37-638, 37-642, 37-648, 37-649, 37-695, 37-704, 37-715, 37-727, 37-743, 37-755, 37-926, 41-787, 44- |
| | 717, 69-870, 88-821, 91-611, 94-760, 109-735, 134-842, 153-246, 178-835, 192-930, 206-905, 229-493, 240-825, |
| | 255-785, 258-513, 258-927, 280-724, 287-1158, 288-905, 298-950, 417-1068, 428-1046, 445-1227, 450-1149, 456- |
| | 892, 530-1335, 615-1157, 619-1163, 622-1491, 651-1167, 672-1383, 686-1302, 687-1248, 730-973, 743-1494, 757- |
| | 1438, 781-1350, 846-1489, 852-1456, 863-1484, 863-1486, 870-1101, 936-1291, 973-1495, 988-1474, 997-1495, |
| | 1016-1420, 1016-1482, 1044-1482, 1180-1438, 1191-1495, 1214-1495, 1238-1495, 1243-1445 |

TABLE 5

| Polynucleotide | Incyte | Representative |
|---|---|---|
| SEQ ID NO: | Project ID: | Library |
| 24 | 5771933CB1 | OVARTUT01 |
| 25 | 70475510CB1 | THP1AZS08 |
| 26 | 566361CB1 | BRAHTDR04 |
| 27 | 71969340CB1 | BRAIFER05 |
| 28 | 6772808CB1 | BRAUNOR01 |
| 29 | 60137669CB1 | KIDEUNE02 |
| 30 | 1987928CB1 | LUNGNON07 |
| 31 | 7268131CB1 | BRAXDIC01 |
| 32 | 7285339CB1 | BONTNOT01 |
| 33 | 7495197CB1 | BRAMNOT01 |
| 34 | 3954126CB1 | BRAWTDR02 |
| 35 | 7499693CB1 | KIDETXF05 |
| 36 | 2187465CB1 | HIPOAZT01 |
| 37 | 3718011CB1 | PLACFER01 |
| 38 | 7500509CB1 | LUNGTUT08 |
| 39 | 7497865CB1 | SPLNTUE01 |
| 40 | 3116578CB1 | MIXDTME01 |
| 41 | 2797803CB1 | NPOLNOT01 |
| 42 | 5433453CB1 | BRSTTMC01 |
| 43 | 6246071CB1 | TESTNOT17 |
| 44 | 7500557CB1 | NPOLNOT01 |
| 45 | 6978182CB1 | BRAHTDR03 |
| 46 | 1985321CB1 | LUNGAST01 |

TABLE 6

| Library | Vector | Library Description |
|---|---|---|
| BONTNOT01 | pINCY | Library was constructed using RNA isolated from tibial |
| | | periosteum removed from a 20-year-old Caucasian male during a |
| | | hemipelvectomy with amputation above the knee. Pathology |
| | | for the associated tumor tissue indicated partially necrotic |
| | | and cystic osteoblastic grade 3 osteosarcoma (post-chemotherapy). |
| | | Family history included osteogenesis imperfecta, closed fracture, |
| | | and type II diabetes. |
| BRAHTDR03 | PCDNA2.1 | This random primed library was constructed using RNA isolated |
| | | from archaecortex, anterior hippocampus tissue removed |
| | | from a 55-year-old Caucasian female who died from cholangiocarcinoma. |
| | | Pathology indicated mild meningeal fibrosis predominately over |
| | | the convexities, scattered axonal spheroids in the white matter |
| | | of the cingulate cortex and the thalamus, and a few scattered |
| | | neurofibrillary tangles in the entorhinal cortex and the |
| | | periaqueductal gray region. Pathology for the associated tumor |
| | | tissue indicated well-differentiated cholangiocarcinoma of the |
| | | liver with residual or relapsed tumor. Patient history included |
| | | cholangiocarcinoma, post-operative Budd-Chiari syndrome, |
| | | biliary ascites, hydrothorax, dehydration, malnutrition, |
| | | oliguria and acute renal failure. Previous surgeries included |
| | | cholecystectomy and resection of 85% of the liver. |
| BRAHTDR04 | PCDNA2.1 | This random primed library was constructed using RNA isolated |
| | | archaecortex, anterior hippocampus tissue removed from |
| | | a 55-year-old Caucasian female who died from cholangiocarcinoma. |
| | | Pathology indicated mild meningeal fibrosis predominately over |
| | | the convexities, scattered axonal spheroids in the white matter |
| | | of the cingulate cortex and the thalamus, and a few scattered |
| | | neurofibrillary tangles in the entorhinal cortex and the |
| | | periaqueductal gray region. Pathology for the associated tumor |
| | | tissue indicated well-differentiated cholangiocarcinoma of the |
| | | liver with residual or relapsed tumor. Patient history included |
| | | cholangiocarcinoma, post-operative Budd-Chiari syndrome, |
| | | biliary ascites, hydrothorax, dehydration, malnutrition, |
| | | oliguria and acute renal failure. Previous surgeries included |
| | | cholecystectomy and resection of 85% of the liver. |
| BRAIFER05 | pINCY | Library was constructed using RNA isolated from brain tissue |
| | | removed from a Caucasian male fetus who was stillborn |
| | | with a hypoplastic left heart at 23 weeks' gestation. |
| BRAMNOT01 | pINCY | Library was constructed using RNA isolated from medulla |
| | | tissue removed from the brain of a 35-year-old Caucasian male |
| | | who died from cardiac failure. Pathology indicated moderate |
| | | leptomeningeal fibrosis and multiple microinfarctions of |
| | | the cerebral neocortex. Microscopically, the cerebral |
| | | hemisphere revealed moderate fibrosis of the leptomeninges with |
| | | focal calcifications. There was evidence of shrunken and |
| | | slightly eosinophilic pyramidal neurons throughout the |
| | | cerebral hemispheres. In addition, scattered throughout the |
| | | cerebral cortex, there were multiple small microscopic |
| | | areas of cavitation with surrounding, gliosis. Patient |
| | | history included dilated cardiomyopathy, congestive heart |
| | | failure, cardiomegaly and an enlarged spleen and liver. |
| BRAUNOR01 | pINCY | This random primed library was constructed using RNA |
| | | isolated from striatum, globus pallidus and posterior putamen |
| | | tissue removed from an 81-year-old Caucasian female who |
| | | died from a hemorrhage and ruptured thoracic aorta due to |
| | | atherosclerosis. Pathology indicated moderate atherosclerosis |
| | | involving the internal carotids, bilaterally; |
| | | microscopic infarcts of the frontal cortex and hippocampus; |
| | | and scattered diffuse amyloid plaques and neurofibrillary |
| | | tangles, consistent with age. Grossly, the leptomeninges |
| | | showed only mild thickening and hyalinization along the |
| | | superior sagittal sinus. The remainder of the leptomeninges |
| | | was thin and contained some congested blood vessels. |
| | | Mild atrophy was found mostly in the frontal poles and |
| | | lobes, and temporal lobes, bilaterally. Microscopically, |
| | | there were pairs of Alzheimer type II astrocytes within |
| | | the deep layers of the neocortex. There was increased |
| | | satellitosis around neurons in the deep gray matter in |
| | | the middle frontal cortex. The amygdala contained rare |
| | | diffuse plaques and neurofibrillary tangles. The posterior |
| | | hippocampus contained a microscopic area of cystic |
| | | cavitation with hemosiderin-laden macrophages surrounded |
| | | by reactive gliosis. Patient history included sepsis, |
| | | cholangitis, post-operative atelectasis, pneumonia CAD, |
| | | cardiomegaly due to left ventricular hypertrophy, |
| | | splenomegaly, arteriolonephrosclerosis, nodular colloidal |
| | | goiter, emphysema, CHF, hypothyroidism, and |
| | | peripheral vascular disease. |
| BRAWTDR02 | PCDNA2.1 | This random primed library was constructed using RNA |
| | | isolated from dentate nucleus tissue removed from a 55-year-old |
| | | Caucasian female who died from cholangiocarcinoma. Pathology |
| | | indicated mild meningeal fibrosis predominately over the |
| | | convexities, scattered axonal spheroids in the white matter |
| | | of the cingulate cortex and the thalamus, and a few |
| | | scattered neurofibrillary tangles in the entorhinal cortex |
| | | and the periaqueductal gray region. Pathology for the |
| | | associated tumor tissue indicated well-differentiated |
| | | cholangiocarcinoma of the liver with residual or relapsed tumor. |
| | | Patient history included cholangiocarcinoma, post-operative |
| | | Budd-Chiari syndrome, biliary ascites, hydrothorax, |
| | | dehydration, malnutrition, oliguria and acute renal failure. |
| | | Previous surgeries included cholecystectomy and resection |
| | | of 85% of the liver. |
| BRAXDIC01 | pINCY | This large size-fractionated library was constructed using |
| | | pooled cDNA from two donors. cDNA was generated using |
| | | mRNA isolated from diseased brain tissue removed from the |
| | | left frontal lobe of a 27-year-old Caucasian male (donor A) |
| | | during a brain lobectomy and from superior temporal cortex |
| | | tissue removed from the brain of a 35-year-old Caucasian |
| | | male (donor B) who died from cardiac failure. Pathology (A) |
| | | indicated a focal deep white matter lesion, characterized |
| | | by marked gliosis, calcifications, and hemosiderin-laden |
| | | macrophages, consistent with a remote perinatal injury. This |
| | | tissue also showed mild to moderate generalized gliosis, |
| | | predominantly subpial and subcortical, consistent with |
| | | chronic seizure disorder. The left temporal lobe, including |
| | | the mesial temporal structures, showed focal, marked |
| | | pyramidal cell loss and gliosis in hippocampal sector CA1, |
| | | consistent with mesial temporal sclerosis. GFAP was |
| | | positive for astrocytes. Pathology (B) indicated moderate |
| | | leptomeningeal fibrosis and multiple microinfarctions of |
| | | the cerebral neocortex. There was evidence of shrunken and |
| | | slightly eosinophilic pyramidal neurons throughout the |
| | | cerebral hemispheres. |
| | | Donor A presented with intractable epilepsy, focal epilepsy, |
| | | hemiplegia, and an unspecified brain injury. Patient |
| | | history (A) included cerebral palsy, abnormality of gait, |
| | | and depressive disorder. Patient history included dilated |
| | | cardiomyopathy, congestive heart failure, and cardiomegaly |
| | | (B). Patient medications included minocycline hydrochloride, |
| | | Tegretol, phenobarbital, Pepcid, and Pevaryl (A) and Simethicone, |
| | | Lasix, Digoxin, Colace, Zantac, Captopril, and Vasotec (B). |
| BRSTTMC01 | pINCY | This large size-fractionated library was constructed using |
| | | pooled cDNA from four donors. cDNA was generated using |
| | | mRNA isolated from diseased breast tissue removed from a |
| | | 40-year-old Caucasian female (donor A) during a bilateral |
| | | reduction mammoplasty; from breast tissue removed from a |
| | | 46-year-old Caucasian female (donor B) during unilateral |
| | | extended simple mastectomy with breast reconstruction; |
| | | from breast tissue removed from a 56-year-old Caucasian female |
| | | (donor C) during unilateral extended simple mastectomy with |
| | | open breast biopsy; and from breast tissue removed from a |
| | | 57-year-old Caucasian female (donor D) during a unilateral |
| | | extended simple mastectomy. Pathology indicated bilateral |
| | | mild fibrocystic and proliferative changes (A); deep |
| | | fascia was negative for tumor (B); non-proliferative fibrocystic |
| | | change (C); and benign fat replaced breast parenchyma (D). |
| | | Pathology for the matched tumor tissue (B) indicated |
| | | invasive grade 3 adenocarcinoma, ductal type, with apocrine |
| | | features. Pathology for the matched tumor tissue (C) |
| | | indicated invasive grade 3 ductal adenocarcinoma. Pathology |
| | | for the matched tumor tissue (D) indicated residual |
| | | microscopic infiltrating grade 3 ductal adenocarcinoma and |
| | | extensive grade 2 intraductal carcinoma. Patient history |
| | | included breast hypertrophy and pure hypercholesterolemia |
| | | (A); breast cancer (B); chronic airway obstruction and |
| | | emphysema (C); and benign hypertension, hyperlipidemia, |
| | | cardiac dysrhythmia, a benign colon neoplasm, a solitary |
| | | breast cyst, and a breast neoplasm of uncertain behavior |
| | | (D). Previous surgeries included open breast biopsy (B). |
| | | Donor B's medications included Cytoxan and Adriamycin. |
| HIPOAZT01 | PSPORT1 | Library was constructed from RNA isolated from diseased |
| | | hippocampus tissue removed from the brain of a 74-year-old |
| | | Caucasian male who died from Alzheimer's disease. |
| KIDETXF05 | PCMV-ICIS | Library was constructed using RNA isolated from a treated, |
| | | transformed embryonal cell line (293-EBNA) derived from |
| | | kidney epithelial tissue. The cells were treated with |
| | | 5-aza-2′-deoxycytidine (5AZA) for 72 hours and Trichostatin |
| | | A for 24 hours and transformed with adenovirus 5 DNA. |
| KIDEUNE02 | pINCY | This 5′ biased random primed library was constructed using |
| | | RNA isolated from an untreated transformed embryonal cell line |
| | | (293-EBNA) derived from kidney epithelial tissue (Invitrogen). |
| | | The cells were transformed with adenovirus 5 DNA. |
| LUNGAST01 | PSPORT1 | Library was constructed using RNA isolated from the lung tissue |
| | | of a 17-year-old Caucasian male, who died from head trauma. |
| | | Patient history included asthma. |
| LUNGNON07 | pINCY | This normalized lung tissue library was constructed from 5.1 |
| | | million independent clones from a lung tissue library. |
| | | Starting RNA was made from RNA isolated from lung tissue. The |
| | | library was normalized in two rounds using conditions adapted |
| | | from Soares et al., PNAS (1994) 91: 9228-9232 and Bonaldo |
| | | et al., Genome Research (1996) 6: 791, except that a significantly |
| | | longer (48 hours/round) reannealing hybridization was used. |
| LUNGTUT08 | pINCY | Library was constructed using RNA isolated from lung tumor |
| | | tissue removed from a 63-year-old Caucasian male during a |
| | | right upper lobectomy with fiberoptic bronchoscopy. Pathology |
| | | indicated a grade 3 adenocarcinoma. Patient history included |
| | | atherosclerotic coronary artery disease, an acute myocardial |
| | | infarction, rectal cancer, an asymtomatic abdominal aortic |
| | | aneurysm, tobacco abuse, and cardiac dysrhythmia. Family history |
| | | included congestive heart failure, stomach cancer, and lung cancer, |
| | | type II diabetes, atherosclerotic coronary artery disease, and |
| | | an acute myocardial infarction. |
| MIXDTME01 | PBK-CMV | This 5′ biased random primed library was constructed using |
| | | pooled cDNA from five donors. cDNA was generated using |
| | | mRNA isolated from small intestine tissue removed from a |
| | | Caucasian male fetus (donor A), who died at 23 weeks' |
| | | gestation from premature birth; from colon epithelium tissue |
| | | removed from a 13-year-old Caucasian female (donor B) who died |
| | | from a motor vehicle accident; from diseased gallbladder tissue |
| | | removed from a 58-year-old Caucasian female (donor C) during |
| | | cholecystectomy and partial parathyroidectomy; from stomach |
| | | tissue removed from a 68-year-old Caucasian female (donor D) |
| | | during a partial gastrectomy; and from breast skin removed from |
| | | a 71-year-old Caucasian female (donor E) during a unilateral |
| | | extended simple mastectomy. For donor C, pathology indicated |
| | | chronic cholecystitis and cholelithiasis. The patient presented |
| | | with abdominal pain and benign parathyroid neoplasm. Patient |
| | | medications included Capoten, Catapres, Norvasc, Synthroid, |
| | | and Xanax. For donor D, pathology indicated the uninvolved |
| | | stomach tissue showed mild chronic gastritis. Patient medications |
| | | included Prilosec, zidoxin, Metamucil, calcium, and vitamins. |
| | | Donor E presented with malignant breast neoplasm and induration. |
| | | Patient medications included insulin, aspirin, and beta carotene. |
| NPOLNOT01 | pINCY | Library was constructed using RNA isolated from nasal polyp |
| | | tissue removed from a 78-year-old Caucasian male during a nasal |
| | | polypectomy. Pathology indicated a nasal polyp and striking |
| | | eosinophilia. Patient history included asthma and nasal polyps. |
| OVARTUT01 | PSPORT1 | Library was constructed using RNA isolated from ovarian tumor |
| | | tissue removed from a 43-year-old Caucasian female during removal |
| | | of the fallopian tubes and ovaries. Pathology indicated grade 2 |
| | | mucinous cystadenocarcinoma involving the entire left ovary. |
| | | Patient history included mitral valve disorder, pneumonia, and |
| | | viral hepatitis. Family history included atherosclerotic coronary |
| | | artery disease, pancreatic cancer, stress reaction, cerebrovascular |
| | | disease, breast cancer, and uterine cancer. |
| PLACFER01 | pINCY | The library was constructed using RNA isolated from placental |
| | | tissue removed from a Caucasian fetus, who died after |
| | | 16 weeks' gestation from fetal demise and hydrocephalus. |
| | | Patient history included umbilical cord wrapped around the |
| | | head (3 times) and the shoulders (1 time). Serology was |
| | | positive for anti-CMV. Family history included multiple |
| | | pregnancies and live births, and an abortion. |
| SPLNTUE01 | PCDNA2.1 | This 5′ biased random primed library was constructed |
| | | using RNA isolated from spleen tumor tissue removed from |
| | | a 28-year-old male during total splenectomy. Pathology |
| | | indicated malignant lymphoma, diffuse large cell type, |
| | | B-cell phenotype with abundant reactive T-cells and marked |
| | | granulomatous response involving the spleen, where it |
| | | formed approximately 45 nodules, liver, and multiple lymph |
| | | nodes. |
| TESTNOT17 | pINCY | Library was constructed from testis tissue removed from a |
| | | 26-year-old Caucasian male who died from head trauma due |
| | | to a motor vehicle accident. Serologies were negative. |
| | | Patient history included a hernia at birth, tobacco use |
| | | (1½ ppd), marijuana use, and daily alcohol |
| | | use (beer and hard liquor). |
| THP1AZS08 | PSPORT1 | This subtracted THP-1 promonocyte cell line library was |
| | | constructed using 5.76 million clones from a 5-aza-2′- |
| | | deoxycytidine (AZ) treated THP-1 cell library. Starting |
| | | RNA was made from THP-1 promonocyte cells treated for three |
| | | days with 0.8 micromolar AZ. The donor had acute monocytic |
| | | leukemia The hybridization probe for subtraction was |
| | | derived from a similarly constructed library, made from |
| | | 1 microgram of polyA RNA isolated from untreated THP-1 cells. |
| | | 5.76 million clones from the AZ-treated THP-1 cell library |
| | | were then subjected to two rounds of subtractive |
| | | hybridization with 5 million clones from the untreated |
| | | THP-1 cell library. Subtractive hybridization conditions were |
| | | based on the methodologies of Swaroop et al., NAR (1991) |
| | | 19: 1954, and Bonaldo et al., Genome Research (1996) 6: 791. |

TABLE 7

| Program | Description | Reference | Parameter Threshold |
|---|---|---|---|
| ABI | A program that removes | Applied Biosystems, | |
| FACTURA | vector sequences and masks | Foster City, CA. | |
| | ambiguous bases in | | |
| | nucleic acid sequences. | | |
| ABI/ | A Fast Data Finder | Applied Biosystems, | Mismatch <50% |
| PARACEL FDF | useful in comparing and | Foster City, CA; | |
| | annotating amino acid or | Paracel Inc., | |
| | nucleic acid sequences. | Pasadena, CA. | |
| ABI | A program that assembles | Applied Biosystems, | |
| AutoAssembler | nucleic acid sequences. | Foster City, CA. | |
| BLAST | A Basic Local Alignment | Altschul, S. F. et al. (1990) | ESTs: Probability value = |
| | Search Tool useful in | J. Mol. Biol. 215: 403-410; | 1.0E−8 or less; Full |
| | sequence similarity search | Altschul, S. F. et al. (1997) | Length sequences: |
| | for amino acid and nucleic | Nucleic Acids Res. 25: 3389-3402. | Probability value = |
| | acid sequences. BLAST | | 1.0E−10 or less |
| | includes five functions: | | |
| | blastp, blastn, blastx, | | |
| | tblastn, and tblastx. | | |
| FASTA | A Pearson and Lipman | Pearson, W. R. and D. J. Lipman | ESTs: fasta E value = |
| | algorithm that searches for | (1988) Proc. Natl. Acad Sci. USA | 1.06E−6; |
| | similarity between a query | 85: 2444-2448; Pearson, W.R. | Assembled ESTs: fasta |
| | sequence and a group of | (1990) Methods Enzymol. 183: 63-98; | Identity = 95% or |
| | sequences of the same type. | and Smith, T. F. and M. S. Waterman | greater and Match |
| | FASTA comprises as | (1981) Adv. Appl. Math. 2: 482-489. | length = 200 bases |
| | least five functions: fasta, | | or greater; fastx E |
| | tfasta, fastx, tfastx, and. | | value = 1.0E−8 or |
| | ssearch. | | less; |
| | | | Full Length sequences: |
| | | | fastx score = 100 or |
| | | | greater |
| BLIMPS | A BLocks IMProved | Henikoff, S. and J. G. Henikoff (1991) | Probability value = |
| | Searcher that matches a | Nucleic Acids Res. 19: 6565-6572; | 1.0E−3 or less |
| | sequence against those | Henikoff, J. G. and S. Henikoff (1996) | |
| | in BLOCKS, PRINTS, | Methods Enzymol. 266: 88-105; and | |
| | DOMO, PRODOM, and PFAM | Attwood, T. K. et al. (1997) J. Chem. | |
| | databases to search | Inf. Comput. Sci. 37: 417-424. | |
| | for gene families, sequence | | |
| | homology, and structural | | |
| | fingerprint regions. | | |
| HMMER | An algorithm for searching | Krogh, A. et al. (1994) J. Mol. Biol. | PFAM, INCY, SMART or |
| | a query sequence against | 235: 1501-1531; Sonnhammer, E. L. | TIGRFAM hits: Probability |
| | hidden Markov model (HMM)- | L. et al. (1988) Nucleic Acids Res. | value = 1.0E−3 or less; |
| | based databases of | 26: 320-322; Durbin, R. et al. | Signal peptide hits: |
| | protein family consensus | (1998) Our World View, in a Nutshell, | Score = 0 or greater |
| | sequences, such as PFAM, | Cambridge Univ. Press, pp. 1-350. | |
| | INCY, SMART and TIGRFAM. | | |
| ProfileScan | An algorithm that searches | Gribskov, M. et al. (1988) CABIOS | Normalized quality score ≧ |
| | for structural and | 4: 61-66; Gribskov, M. et al. | GCG specified “HIGH” |
| | sequence motifs in protein | (1989) Methods Enzymol. 183: 146-159; | value for that particular |
| | sequences that match | Bairoch, A. et al. (1997) Nucleic | Prosite motif. |
| | sequence patterns defined | Acids Res. 25: 217-221. | Generally, score = |
| | in Prosite. | | 1.4-2.1. |
| Phred | A base-calling algorithm | Ewing, B. et al. (1998) Genome Res. | |
| | that examines automated | 8: 175-185; Ewing, B. and P. Green | |
| | sequencer traces with | (1998) Genome Res. 8: 186-194. | |
| | high sensitivity and | | |
| | probability. | | |
| Phrap | A Phils Revised Assembly | Smith, T. F. and M.S. Waterman (1981) | Score = 120 or greater; |
| | Program including | Adv. Appl. Math. 2: 482-489; Smith, | Match length = 56 or |
| | SWAT and CrossMatch, | T. F. and M.S. Waterman (1981) J. Mol. | greater |
| | programs based on efficient | Biol. 147: 195-197; and Green, P., | |
| | implementation of the | University of Washington, Seattle, WA. | |
| | Smith-Waterman algorithm, | | |
| | useful in searching | | |
| | sequence homology and | | |
| | assembling DNA sequences. | | |
| Consed | A graphical tool for | Gordon, D. et al. (1998) Genome Res. | |
| | viewing and editing Phrap | 8: 195-202. | |
| | assemblies. | | |
| SPScan | A weight matrix analysis | Nielson, H. et al. (1997) Protein | Score = 3.5 or greater |
| | program that scans protein | Engineering 10: 1-6; Claverie, | |
| | sequences for the presence | J. M. and S. Audic (1997) CABIOS 12: | |
| | of secretory signal | 431-439. | |
| | peptides. | | |
| TMAP | A program that uses | Persson, B. and P. Argos (1994) J. | |
| | weight matrices to | Mol. Biol. 237: 182-192; Persson, | |
| | delineate transmembrane | B. and P. Argos (1996) Protein Sci. | |
| | segments on protein sequences | 5: 363-371. | |
| | and determine orientation. | | |
| TMHMMER | A program that uses a | Sonnhammer, E. L. et al. (1998) | |
| | hidden Markov model (HMM) | Proc. Sixth Intl. Conf. On Intelligent | |
| | to delineate transmembrane | Systems for Mol. Biol., Glasgow et al., | |
| | segments on protein | eds., The Am. Assoc. for Artificial | |
| | sequences and determine | Intelligence (AAAI) Press, | |
| | orientation. | Menlo Park, CA, and MTT Press, | |
| | | Cambridge, MA, pp. 175-182. | |
| Motifs | A program that searches | Bairoch, A. et al. (1997) Nucleic | |
| | amino acid sequences for | Acids Res. 25: 217-221; Wisconsin | |
| | patterns that matched | Package Program Manual, version 9, | |
| | those defined in Prosite. | page M51-59, Genetics Computer | |
| | | Group, Madison, WI. | |

1. An isolated polypeptide selected from the group consisting of:
a) a polypeptide comprising an amino acid sequence selected from the group consisting of SEQ ID NO:1-23,
b) a polypeptide comprising a naturally occurring amino acid sequence at least 90% identical to an amino acid sequence selected from the group consisting of SEQ ID NO:1-4, SEQ ID NO:6-10, SEQ ID NO:12-14, SEQ ID NO:17, and SEQ ID NO:19-23,
c) a polypeptide comprising a naturally occurring amino acid sequence at least 91% identical to the amino acid sequence of SEQ ID NO:18,
d) a polypeptide comprising a naturally occurring amino acid sequence at least 92% identical to the amino acid sequence of SEQ ID NO:11,
e) a polypeptide comprising a naturally occurring amino acid sequence at least 94% identical to the amino acid sequence of SEQ ID NO:5,
f) a polypeptide comprising a naturally occurring amino acid sequence at least 98% identical to the amino acid sequence of SEQ ID NO:16,
g) a polypeptide comprising a naturally occurring amino acid sequence at least 99% identical to the amino acid sequence of SEQ ID NO:15,
h) a biologically active fragment of a polypeptide having an amino acid sequence selected from the group consisting of SEQ ID NO:1-23, and
i) an immunogenic fragment of a polypeptide having an amino acid sequence selected from the group consisting of SEQ ID NO:1-23.

2. An isolated polypeptide of claim 1 consisting an amino acid sequence selected from the group consisting of SEQ ID NO:1-23.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:24-46.

6. A recombinant polynucleotide comprising a promoter sequence operably linked to a polynucleotide of claim 3.

7. A cell transformed with a recombinant polynucleotide of claim 6.

8. (CANCELED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-23.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:24-46,
b) a polynucleotide comprising a naturally occurring polynucleotide sequence at least 90% identical to a polynucleotide sequence selected from the group consisting of SEQ ID NO:24-46,
c) a polynucleotide complementary to a polynucleotide of a),
d) a polynucleotide complementary to a polynucleotide of b), and
e) an RNA equivalent of a)-d).

13. (CANCELED)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELED)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-23.

19. (CANCELED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (CANCELED)

22. (CANCELED)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (CANCELED)

25. (CANCELED)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELED)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-101. (CANCELED)


SEQUENCE LISTING (46 sequences)

SEQ ID NO: 1 - protein, 423 aa, Homo sapiens
  misc_feature: Incyte ID No 5771933CD1
MVFAFWKVFLILSCLAGQVSVVQVTIPDGFVNVTVGSNVTLICIYTTTVASREQLSIQWSFFHKKEMEPISHSSCLSTEGMEEKAVSQCLKMTHARDARGRCSWTSEIYFSQGGQAVAIGQFKDRITGSNDPGNASITISHMQPADSGIYICDVNNPPDFLGQNQGILNVSVLVKPSKPLCSVQGRPETGHTISLSCLSALGTPSPVYYWHKLEGRDIVPVKENFNPTTGILVIGNLTNFEQGYYQCTAINRLGNSSCEIDLTSSHPEVGIIVGALIGSLVGAAIIISVVCFARNKAKAKAKERNSKTIAELEPMTKINPRGEGEAMPREDATQLEVTLPSSIHETGPDTIQEPDYEPKPTQEPAPEPAPGSEPMAVPDLDIELELEPETQSELEPEPEPEPESEPGVVVEPLSEDEKGVVKA

SEQ ID NO: 2 - protein, 972 aa, Homo sapiens
  misc_feature: Incyte ID No 70475510CD1
MPPVYASEYVLPLQGGGSGEEQLYADFPELDLSQLDASDFDSATCFGELQWCPENSETEPNQYSPDDSELFQIDSENEALLAELTKTLDDIPEDDVGLAAFPALDGGDALSCTSASPAPSSAPPSPAPEKPSAPAPEVDELSLADSTQDKKAPMMQSQSRSCTELHKHLTSAQCCLQDRGLQPPCLQSPRLPAKEDKEPGEDCPSPQPAPASPRDSLALGRADPGAPVSQEDMQAMVQLIRYMHTYCLPQRKLPPQTPEPLPKACSNPSQQVRSRPWSRHHSKASWAEFSILRELLAQDVLCDVSKPYRLATPVYASLTPRSRPRPPKDSQASPGRPSSVEEVRIAASPKSTGPRPSLRPLRLEVKREVRRPARLQQQEEEDEEEEEEEEEEEKEEEEEWGRKRPGRGLPWTKLGRKLESSVCPVRRSRRLNPELGPWLTFADEPLVPSEPQGALPSLCLAPKAYDVERELGSPTDEDSGQDQQLLRGPQIPALESPCESGCGDMDEDPSCPQLPPRDSPRCLMLALSQSDPTFGKKSFEQTLTVELCGTAGLTPPTTPPYKPTEEDPFKPDIKHSLGKEIALSLPSPEGLSLKATPGAAHKLPKKHPERSELLSHLRHATAQPASQAGQKRPFSCSFGDHDYCQVLRPEGVLQRKVLRSWEPSGVHLEDWPQQGAPWAEAQAPGREEDRSCDAGAPPKDSTLLRDHEIRASLTKHFGLLETALEEEDLASCKSPEYDTVFEDSSSSSGESSFLPEEEEEEGEEEEEDDEEEDSGVSPTCSDHCPYQSPPSKANRQLCSRSRSSSGSSPCHSWSPATRRNFRCESRGPCSDRTPSIRHARKRREKAIGEGRVVYIQNLSSDMSSRELKRRFEVFGEIEECEVLTRNRRGEKYGFITYRCSEHAALSLTKGAALRKRNEPSFQLSYGGLRHFCWPRYTDYDSNSEEALPASGKSKYEAMDFDSLLKEAQQSLH

SEQ ID NO: 3 - protein, 827 aa, Homo sapiens
  misc_feature: Incyte ID No 566361CD1
MASADKNGGSVSSVSSSRLQSRKPPNLSITIPPPEKETQAPGEQDSMLPERKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHALHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 4 - protein, 828 aa, Homo sapiens
  misc_feature: Incyte ID No 71969340CD1
MAGRGWGALWVCVAAATLLHAGGLARADCWLIEGDKGFVWLAICSQNQPPYEAIPQQINSTIVDLRLNENRIRSVQYASLSRFGNLTYLNLTKNEIGYIEDGAFSGQFNLQVLQLGYNRLRNLTEGMLRGLGKLEYLYLQANLIEVVMASSFWECPNIVNIDLSMNRIQQLNSGTFAGLAKLSVCELYSNPFYCSCELLGFLRWLAAFTNATQTYDRMQCESPPVYSGYYLLGQGRRGHRSILSKLQSVCTEDSYAAEVVGPPRPASGRSQPGRSPPPPPPPEPSDMPCADDECFSGDGTTPLVALPTLATQAEARPLIKVKQLTQNSATITVQLPSPFHRMYTLEHFNNSKASTVSRLTKAQEEIRLTNLFTLTNYTYCVVSTSAGLRHNHTCLTICLPRLPSPPGPVPSPSTATHYIMTILGCLFGMVLVLGAVYYCLRRRRRQEEKHKKAASAAAAGSLKKTIIELKYGPELEAPGLAPLSQGPLLGPEAVTRIPYLPAAGEVEQYKLVESADTPKASKGSYMEVRTGDPPERRDCELGRPGPDSQSSVAEISTIAKEVDKVNQIINNCIDALKSESTSFQGVKSGPVSVAEPPLVLLSEPLAAKHGFLAPGYKDAFGHSLQRHHSVEAAGPPRASTSSSGSVRSPRAFRAEAVGVHKAAAAEAKYIEKGSPAADAILTVTPAAAVLRAEAEKGRQYGEHRHSYPGSHPAEPPAPPGPPPPPPHEGLGRKASILEPLTRPRPRDLAYSQLSPQYHSLSYSSSPEYTCRASQSIWERFRLSRRRHKEEEEFMAAGHALRKKVQFAKDEDLHDILDYWKGVSAQHKS

SEQ ID NO: 5 - protein, 1168 aa, Homo sapiens
  misc_feature: Incyte ID No 6772808CD1
MGKVGAGGGSQARLSALLAGAGLLILCAPGVCGGGSCCPSPHPSSAPRSASTPRGFSHQGRPGRAPATPLPLVVRPLFSVAPGDRALSLERARGTGASMAVAARSGRRRRSGADQEKAERGEGASRSPRGVLRDGGQQEPGTRERDPDKATRFRMEELRLTSTTFALTGDSAHNQAMVHWSGHNSSVILILTKLYDYNLGSITESSLWRSTDYGTTYEKLNDKVGLKTILSYLYVCPTNKRKIMLLTDPEIESSLLISSDEGATYQKYRLNFYIQSLLFHPKQEDWILAYSQDQKLYSSAEFGRRWQLIQEGVVPNRFYWSVMGSNKEPDLVHLEARTVDGHSHYLTCRMQNCTEANRNQPFPGYIDPDSLIVQDHYVFVQLTSGGRPHYYVSYRRNAFAQMKLPKYALPKDMHVISTDENQVFAAVQEWNQNDTYNLYISDTRGVYFTLALENVQSSRGPEGNIMIDLYEVAGIKGMFLANKKIDNQVKTFITYNKGRDWRLLQAPDTDLRGDPVHCLLPYCSLHLHLKVSENPYTSGIIASKDTAPSIIVASGNIGSELSDTDISMFVSSDAGNTWRQIFEEEHSVLYLDQGGVLVAMKHTSLPIRHLWLSFDEGRSWSKYSFTSIPLFVDGVLGEPGEETLIMTVFGHFSHRSEWQLVKVDYKSIFDRRCAEEDYRPWQLHSQGEACIMGAKRIYKKRKSERKCMQGKYAGAMESEPCVCTEADFDCDYGYERHSNGQCLPAFWFNPSSLSKDCSLGQSYLNSTGYRKVVSNNCTDGVREQYTAKPQKCPGKAPRGLRIVTADGKLTAEQGHNVTLMVQLEEGDVQRTLIQVDFGDGIAVSYVNLSSMEDGIKHAYQNVGIFRVTVQVDNSLGSDSAVLYLHVTCPLEHVHLSLPFVTTKNKEVNATAVLWPSQVGTLTYVWWYGNNTEPLITLEGSISFRFTSEGMNTITVQVSAGNAILQDTKTIAVYEEFRSLRLSFSPNLDDYNPDIPEWRRDIGRVIKKSLVEATGVPGQHILVAVLPGLPTTAELFVLPYQDPAGENKRSTDDLEQISELLIHTLNQNSVHFELKPGVRVLVHAAHLTAAPLVDLTPTHSGSAMLMLLSVVFVGLAVFVIYKFKRRVALPSPPSPSTQPGDSSLRLQRARHATPPSTPKRGSAGAQYAI

SEQ ID NO: 6 - protein, 300 aa, Homo sapiens
  misc_feature: Incyte ID No 60137669CD1
MDIEATNRDYKRPLHEAASMGHRDCVRYLLGRGAAVDCLKKADWTPLMMACTRKNLGVIQELVEHGANPLLKNKDGWNSFHIASREGDPLILQYLLTVCPGAWKTESKIRRTPLHTAAMHGHLEAVKVLLKRCQYEPDYRDNCGVTALMDAIQCGHIDVARLLLDEHGACLSAEDSLGAQALHRAAVTGQDEAIRFLVSELGVDVDVRATSTHLTALHYAAKEGHTSTIQTLLSLGADINSKDEKNRSALHLACAGQHLACAKFLLQSGLKDSEDITGTLAQQLPRRADVLRGSGHSAMT

SEQ ID NO: 7 - protein, 240 aa, Homo sapiens
  misc_feature: Incyte ID No 1987928CD1
MSAAPASNGVFVVIPPNNASGLCPPPAILPTSMCQPPGIMQFEEPPLGAQTPRATQPPDLRPVETFLTGEPKVLGTVQILIGLIHLGFGSVLLMVRRGHVGIFFIEGGVPFWGGACFIISGSLSVAAEKNHTSCLVRSSLGTNILSVMAAFAGTAILLMDFGVTNRDVDRGYLAVLTIFTVLEFFTAVIAMHFGCQAIHAQASAPVIFLPNAFSADFNIPSPAASAPPAYDNVAYAQGVV

SEQ ID NO: 8 - protein, 394 aa, Homo sapiens
  misc_feature: Incyte ID No 7268131CD1
MAASSSEISEMKGVEESPKVPGEGPGHSEAETGPPQVLAGVPDQPEAPQPGPNTTAAPVDSGPKAGLAPETTETPAGASETAQATDLSLSPGGESKANCSPEDPCQETVSKPEVSKEATADQGSRLESAAPPEPAPEPAPQPDPRPDSQPTPKPALQPELPTQEDPTPEILSESVGEKQENGAVVPLQAGDGEEGPAPEPHSPPSKKSPPANGAPPRVLQQLVEEDRMRRAHSGHPGSPRGSLSRHPSSQLAGPGVEGGEGTQKPRDYIILAILSCFCPMWPVNIVAFAYAVMSRNSLQQGDVDGAQRLGRVAKLLSIVALVGGVLIIIASCVINLGGEWGLGTGRGGMEGLARAALLTPAPALSCLSSLPLLCLSLSPPPPVCPSLSSPTVYK

SEQ ID NO: 9 - protein, 340 aa, Homo sapiens
  misc_feature: Incyte ID No 7285339CD1
MAASSSEISEMKGVEESPKVPGEGPGHSEAETGPPQVLAGVPDQPEAPQPGPNTTAAPVDSGPKAGLAPETTETPAGASETAQATDLSLSPGGESKANCSPEDPCQETVSKPEVSKEATADQGSRLESAAPPEPAPEPAPQPDPRPDSQPTPKPALQPELPTQEDPTPEILSESVGEKQENGAVVPLQAGDGEEGPAPEPHSPPSKKSPPANGAPPRVLQQLVEEDRMRRAHSGHPGSPRGSLSRHPSSQLAGPGVEGGEGTQKPRDYIILAILSCFCPMWPVNIVAFAYAVMSRNSLQQGDVDGAQRLGRVAKLLSIVALVGGVLIIIASCVINLGVYK

SEQ ID NO: 10 - protein, 525 aa, Homo sapiens
  misc_feature: Incyte ID No 7495197CD1
MVVASLIILHLSGATKKGTEKQTTSETQKSVQCGTWTKHAEGGIFTSPNYPSKYPPDRECIYIIEAAPRQCIELYFDEKYSIEPSWECKFDHIEVRDGPFGFSPIIGRFCGQQNPPVIKSSGRFLWIKFFADGELESMGFSARYNFTPDPDFKDLGALKPLPACEFEMGGSEGIVESIQIMKEGKATASEAVDCKWYIRAPPRSKIYLRFLDYEMQNSNECKRNFVAVYDGSSSVEDLKAKFCSTVANDVMLRTGLGVIRMWADEGSRNSRFQMLFTSFQEPPCEGNTFFCHSNMCINNTLVCNGLQNCVYPWDENHCKEKRKTSLLDQLTNTSGTVIGVTSCIVIILIIISVIVQIKQPRKKYVQRKSDFDQTVFQEVFEPPHYELCTLRGTGATADFADVADDFENYHKLRRSSSKCIHDHHCGSQLSSTKGSRSNLSTRDASILTEMPTQPGKPLIPPMNRRNILVMKHNYSQDAADACDIDEIEEVPTTSHRLSRHDKAVQRFCLIGSLSKHESEYNTTRV

SEQ ID NO: 11 - protein, 2214 aa, Homo sapiens
  misc_feature: Incyte ID No 3954126CD1
MVANFFKSLILPYIHKLCKGMFTKKLGNTNKNREYRQQKKDQDFPTAGQTKSPKFSYTFKSTVKKIAKCSSTHNLSTEEDEASKEFSLSPTFSYRVAIANGLQKNAKVTNSDNEDLLQELSSIESSYSESLNELRSSTENQAQSTHTMPVRRNRKSSSSLAPSEGSSDGERTLHGLKLGALRKLRKWKKSQECVSSDSELSTMKKSWGIRSKSLDRTVRNPKTNALEPGFSSSGCISQTHDVMEMIFKELQGISQIETELSELRGHVNALKHSIDEISSSVEVVQSEIEQLRTGFVQSRRETRDIHDYIKHLGHMGSKASLRFLNVTEERFEYVESVVYQILIDKMGFSDAPNAIKIEFAQRIGHQRDCPNAKPRPILVYFETPQQRDSVLKKSYKLKGTGIGISTDILTHDIRERKEKGIPSSQTYESMAIKLSTPEPKIKKNNWQSPDDSDEDLESDLNRNSYAVLSKSELLTKGSTSKPSSKSHSARSKNKTANSSRISNKSDYDKISSQLPESDILEKQTTTHYADATPLWHSQSDFFTAKLSRSESDFSKLCQSYSEDFSENQFFTRTNGSSLLSSSDRELWQRKQEGTATLYDSPKDQHLNGGVQGIQGQTETENTETVDSGMSNGMVCASGDRSHYSDSQLSLHEDLSPWKEWNQGADLGLDSSTQEGFDYETNSLFDQQLDVYNKDLEYLGKCHSDLQDDSESYDLTQDDNSSPCPGLDNEPQGQWVGQYDSYQGANSNELYQNQNQLSMMYRSQSELQSDDSEDAPPKSWHSRLSIDLSDKTFSFPKFGSTLQRAKSALEVVWNKSTQSLSGYEDSGSSLMGRFRTLSQSTANESSTTLDSDVYTEPYYYKAEDEEDYTEPVADNETDYVEVMEQVLAKLENRTSITETDEQMQAYDHLSYETPYETPQDEGYDGPADDMVSEEGLEPLNETSAEMEIREDENQNIPEQPVEITKPKRIRPSFKEAALRAYKKQMAELEEKILAGDSSSVDEKARIVSGNDLDASKFSALQVCGGAGGGLYGIDSMPDLRRKKTLPIVRDVAMTLAARKSGLSLAMVIRTSLNNEELKMHVFKKTLQALIYPMSSTIPHNFEVWTATTPTYCYECEGLLWGIARQGMKCLECGVKCHEKCQDLLNADCLQRAAEKSSKHGAEDKTQTIITAMKERMKIREKNRPEVFEVIQEMFQISKEDFVQFTKAAKQSVLDGTSKWSAKITITVVSAQGLQAKDKTGSSDPYVTVQVGKNKRRTKTIFGNLNPVWDEKFYFECHNSTDRIKVRVWDEDDDIKSRVKQHFKKESDDFLGQTIVEVRTLSGEMDVWYNLEKRTDKSAVSGAIRLKINVEIKGEEKVAPYHIQYTCLHENLFHYLTEVKSNGGVKIPEVKGDEAWKVFFDDASQEIVDEFAMRYGIESIYQAMTHFSCLSSKYMCPGVPAVMSTLLANINAFYAHTTVSTNIQVSASDRFAATNFGREKFIKLLDQLHNSLRIDLSKYRENFPASNTERLQDLKSTVDLLTSITFFRMKVLELQSPPKASMVVKDCVRACLDSTYKYIFDNCHELYSQLTDPSKKQDIPREDQGPTTKNLDFWPQLITLMVTIIDEDKTAYTPVLNQFPQELNMGKISAEIMWTLFALDMKYALEEHDNQRLCKSTDYMNLHFKVKWFYNEYVRELPAFKDAVPEYSLWFEPFVMQWLDENEDVSMEFLHGALGRDKKDGFQQTSEHALFSCSVVDVFAQLNQSFEIIKKLECPNPEALSHLMRRFAKTINKVLLQYAAIVSSDFSSHCDKENVPCILMNNIQQLRVQLEKMFESMGGKELDSEASTILKELQVKLSGVLDELSVTYGESFQVIIEECIKQMSFELNQMRANGNTTSNKNSAAMDAEIVLRSLMDFLDKTLSLSAKICEKTVLKRVLKELWKLVLNKIEKQIVLPPLTDQTGPQMIFIAAKDLGQLSKLKEHMIREDARGLTPRQCAIMEVVLATIKQYFHAGGNGLKKNFLEKSPDLQSLRYALSLYTQTTDALIKKFIDTQTSQSRSSKDAVGQISVHVDITATPGTGDHKVTVKVIAINDLNWQTTAMFRPFVEVCILGPNLGDKKRKQGTKTKSNTWSPKYNETFQFILGKENRPGAYELHLSVKDYCFAREDRIIGMTVIQLQNIAEKGSYGAWYPLLKNISMDETGLTILRILSQRTSDDVAKEFVRLKSETRSTEESA

SEQ ID NO: 12 - protein, 487 aa, Homo sapiens
  misc_feature: Incyte ID No 7499693CD1
MALERLCSVLKVLLITVLVVEGIAVAQKTQDGQNIGIKHIPATQCGIWVRTSNGGHFASPNYPDSYPPNKECIYILEAAPRQRIELTFDEHYYIEPSFECRFDHLEVRDGPFGFSPLIDRYCGVKSPPLIRSTGRFMWIKFSSDEELEGLGFRAKYSFIPDPDFTYLGGILNPIPDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEQKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 13 - protein, 405 aa, Homo sapiens
  misc_feature: Incyte ID No 2187465CD1
MNKNTSTVVSPSLLEKDPAFQMITIAKETGLGLKVLGGINRNEGPLVYIQEIIPGGDCYKDGRLKPGDQLVSVNKESMIGVSFEEAKSIITRAKLRLESAWEIAFIRQKSDNIQPENLSCTSLIEASGEYGPQASTLSLFSSPPEILIPKTSSTPKTNNDILSSCEIKTGYNKTVQIPITSENSTVGLSNTDVASAWTENYGLQEKISLNPSVRFKAEKLEMALNYLGIQPTKEQHQALRQQVQADSKGTVSFGDFVQVARNLFCLQLDEVNVGAHEISNILDSQLLPCDSSEADEMERLKCERDDALKEVNTLKEAKAVVEETRALRSRIHLAEAAQRQAHGMEMDYEEVIRLLEAKITELKAQLADYSDQNKVSKAVISSSYHGFLAVVMYPVFIFFSSALLN

SEQ ID NO: 14 - protein, 910 aa, Homo sapiens
  misc_feature: Incyte ID No 3718011CD1
MKKMSRNVLLQMEEEEDDDDGDIVLENLGQTIVPDLGSLESQHDFRTPEFEEFNGKPDSLFFNDGQRRIDFVLVYEDESRKETNKKGTNEKQRRKRQAYESNLICHGLQLEATRSVLDDKLVFVKVHAPWEVLCTYAEIMHIKLPLKPNDLKNRSSAFGTLNWFTKVLSVDESIIKPEQEFFTAPFEKNRMNDFYIVDRDAFFNPATRSRIVYFILSRVKYQVINNVSKFGINRLVNSGIYKAAFPLHDCKFRRQSEDPSCPNERYLLYREWAHPRSIYKKQPLDLIRKYYGEKIGIYFAWLGYYTQMLLLAAVVGVACFLYGYLNQDNCTWSKEVCHPDIGGKIIMCPQCDRLCPFWKLNITCESSKKLCIFDSFGTLVFAVFMGVWVTLFLEFWKRRQAELEYEWDTVELQQEEQARPEYEARCTHVVINEITQEEERIPFTAWGKCIRITLCASAVFFWILLIIASVIGIIVYRLSVFIVFSAKLPKNINGTDPIQKYLTPQTATSITASIISFIIIMILNTIYEKVAIMITNFELPRTQTDYENSLTMKMFLFQFVNYYSSCFYIAFFKGKFVGYPGDPVYWLGKYRNEECDPGGCLLELTTQLTIIMGGKAIWNNIQEVLLPWIMNLIGRFHRVSGSEKITPRWEQDYHLQPMGKLGLFYEYLEMIIQFGFVTLFVASFPLAPLLALVNNILEIRVDAWKLTTQFRRLVPEKAQDIGAWQPIMQGIAILAVVTNAMIIAFTSDMIPRLVYYWSFSVPPYGDHTSYTMEGYINNTLSIFKVADFKNKSKGNPYSDLGNHTTCRYRDFRYPPGHPQEYKHNIYYWHVIAAKLAFIIVMEHVIYSVKFFISYAIPDVSKRTKSKIQREKYLTQKLLHENHLKDMTKNMGVIAERMIEAVDNNLRPKSE

SEQ ID NO: 15 - protein, 327 aa, Homo sapiens
  misc_feature: Incyte ID No 7500509CD1
MRLAVLFSGALLGLLAESTGTTSHRTTKSHKTTTHRTTTTGTTSHGPTTATHNPTTTSHGNVTVHPTSNSTATSQGPSTATHSPATTSHGNATVHPTSNSTATSPGFTSSAHPEPPPPSPSPSPTSKETIGDYTWTNGSQPCVHLQAQIQIRVMYTTQGGGEAWGISVLNPNKTKVQGSCEGAHPHLLLSFPYGHLSFGFMQDLQQKVVYLSYMAVEYNVSFPHAAQWTFSAQNASLRDLQAPLGQSFSCSNSSIILSPAVHLDLLSLRLQAAQLPHTGVFGQSFSCPSDRSILLPLIIGLILLGLLALVLIAFCIIRRRPSAYQAL

SEQ ID NO: 16 - protein, 416 aa, Homo sapiens
  misc_feature: Incyte ID No 7497865CD1
MEATGISLASQLKVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 17 - protein, 635 aa, Homo sapiens
  misc_feature: Incyte ID No 3116578CD1
MSGAGRALAALLLAASVLSAALLAPGGSSGRDAQAAPPRDLDKKRHAELKMDQALLLIHNELLWTNLTVYWKSECCYHCLFQVLVNVPQSPKAGKPSAAAASVSTQHGSILQLNDTLEEKEVCRLEYRFGEFGNYSLLVKNIHNGVSEIACDLAVNEDPVDSNLPVSIAFLIGLAVIIVISFLRLLLSLDDFNNWISKAISSRETDRLINSELGSPSRTDPLDGDVQPATWRLSALPPRLRSVDTFRGIALILMVFVNYGGGKYWYFKHASWNGLTVADLVFPWFVFIMGSSIFLSMTSILQRGCSKFRLLGKIAWRSFLLICIGIIIVNPNYCLGPLSWDKVRIPGVLQRLGVTYFVVAVLELLFAKPVPEHCASERSCLSLRDITSSWPQWLLILVLEGLWLGLTFLLPVPGCPTGYLGPGGIGDFGKYPNCTGGAAGYIDRLLLGDDHLYQHPSSAVLYHTEVAYDPEGILGTINSIVMAFLGVQAGKILLYYKARTKDILIRFTAWCCILGLISVALTKVSENEGFIPVNKNLWSLSYVTTLSSFAFFILLVLYPVVDVKGLWTGTPFFYPGMNSILVYVGHEVFENYFPFQWKLKDNQSHKEHLTQNIVATALWVLIAYILYRKKIFWKI

SEQ ID NO: 18 - protein, 478 aa, Homo sapiens
  misc_feature: Incyte ID No 2797803CD1
MPARSRHRPRLHSGSPPRAPPPPLEALHSGEAGRAPDSDGGSDADSEVGPGSPTRTAEAAEEEMAGPNQLCIRRWTTKHVAVWLKDEGFFEYVDILCNKHRLDGITLLTLTEYDLRSPPLEIKVLGDIKRLMLSVRKLQKIHIDVLEEMGYNSDSPMGSMTPFISALQSTDWLCNGELSHDCDGPITDLNSDQYQYMNGKNKHSVRRLDPEYWKTILSCIYVFIVFGFTSFIMVIVHERVPDMQTYPPLPDIFLDSVPRIPWAFAMTEVCGMILCYIWLLVLLLHKHRSILLRRLCSLMGTVFLLRCFTMFVTSLSVPGQHLQCTGKIYGSVWEKLHRAFAIWSGFGMTLTGVHTCGDYMFSGHTVVLTMLNFFVTEYTPRSWNFLHTLSWVLNLFGIFFILAAHEHYSIDVFIAFYITTRLFLYYHTLANTRAYQQSRRARIWFPMFSFFECNVNGTVPNEYCWPFSKPAIMKRLIG

SEQ ID NO: 19 - protein, 634 aa, Homo sapiens
  misc_feature: Incyte ID No 5433453CD1
MAMWNRPCQRLPQQPLVAEPTAEGEPHLPTGRELTEANRFAYAALCGISLSQLFPEPEHSSFCTEFMAGLVQWLELSEAVLPTMTAFASGLGGEGADVFVQILLKDPILKDDPTVITQDLLSFSLKDGHYDARARVLVCHMTSLLQVPLEELDVLEEMFLESLKEIKEEESEMAEASRKKKENRRKWKRYLLIGLATVGGGTVIGVTGGLAAPLVAAGAATIIGSAGAAALGSAAGIAIMTSLFGAAGAGLTGYKMKKRVGAIEEFTFLPLTEGRQLHITIAVTGWLASGKYRTFSAPWAALAHSREQYCLAWEAKYLMELGNALETILSGLANMVAQEALKYTVLSGIVAALTWPASLLSVANVIDNPWGVCLHRSAEVGKHLAHILLSRQQGRRPVTLIGFSLGARVIYFCLQEMAQEKDCQGIIEDVILLGAPVEGEAKHWEPFRKVVSGRIINGYCRGDWLLSFVYRTSSVQLHVAGLQPVLLQDRRVENVDLTSVVSGHLDYAKQMDAILKAVGIRTKPGWDEKGLLLAPGCLPSEEPRQAAAAASSGETPHQVGQTQGPISGDTSKLAMSTDPSQAQVPVGLDQSEGASLPAAASPERPPICSHGMDPNPLGCPDCACKTQGPSTGLD

SEQ ID NO: 20 - protein, 152 aa, Homo sapiens
  misc_feature: Incyte ID No 6246071CD1
MMQQPRVETDTIGAGEGPQQAVPWSAWVTRHGWVRWWVSHMPPSWIQWWSTSNWRQPLQRLLWGLEGILYLLLALMLCHALFTTGSHLLSSLWPVVAAVWRHLLPALLLLVLSALPALLFTASFLLLFSTLLSLVGLLTSMTHPGDTQDLDQ

SEQ ID NO: 21 - protein, 308 aa, Homo sapiens
  misc_feature: Incyte ID No 7500557CD1
MPARSRHRPRLHSGSPPRAPPPPLEALHSGEAGRAPDSDGGSDADSEVGPGSPTRTAEAAEEEMAGPNQLCIRRWTTKHVAVWLKDEGFFEYVDILCNKHRLDGITLLTLTEYDLRSPPLEIKVLGDIKRLMLSVRKLQKIHIDVLEEMGYNSDSPMGSMTPFISALQSTDWLCNGELSHDCDGPITDLNSDQYQYMNGKNKHSVRRLDPEYWKTILSCIYVFIVFGFTSFIMVIVHERVPDMQTYPPLPDIFLDSVPRIPWAFAMTEVCGMILCYIWLLVLLLHKHRYMAVYGRNYIEPLPFGVALV

SEQ ID NO: 22 - protein, 431 aa, Homo sapiens [sequence omitted]

SEQ ID NO: 23 - protein, 93 aa, Homo sapiens
  misc_feature: Incyte ID No 1985321CD1
MAAFAGTAILLMDFGVTNRDVDRGYLAVLTIFTVLEFFTAVIAMHFGCQAIHAQASAPVIFLPNAFSADFNIPSPAASAPPAYDNVAYAQGVV

SEQ ID NO: 24 - DNA, 1748 bp, Homo sapiens
  misc_feature: Incyte ID No 5771933CB1
acaatggtgttcgcattttggaaggtctttctgatcctaagctgccttgcaggtcaggttagtgtggtgcaagtgaccatcccagacggtttcgtgaacgtgactgttggatctaatgtcactctcatctgcatctacaccaccactgtggcctcccgagaacagctttccatccagtggtctttcttccataagaaggagatggagccaatttctcacagctcgtgcctcagtactgagggtatggaggaaaaggcagtcagtcagtgtctaaaaatgacgcacgcaagagacgctcggggaagatgtagctggacctctgagatttacttttctcaaggtggacaagctgtagccatcgggcaatttaaagatcgaattacagggtccaacgatccaggtaatgcatctatcactatctcgcatatgcagccagcagacagtggaatttacatctgcgatgttaacaaccccccagactttctcggccaaaaccaaggcatcctcaacgtcagtgtgttagtgaaaccttctaagcccctttgtagcgttcaaggaagaccagaaactggccacactatttccctttcctgtctctctgcgcttggaacaccttcccctgtgtactactggcataaacttgagggaagagacatcgtgccagtgaaagaaaacttcaacccaaccaccgggattttggtcattggaaatctgacaaattttgaacaaggttattaccagtgtactgccatcaacagacttggcaatagttcctgcgaaatcgatctcacttcttcacatccagaagttggaatcattgttggggccttgattggtagcctggtaggtgccgccatcatcatctctgttgtgtgcttcgcaaggaataaggcaaaagcaaaggcaaaagaaagaaattctaagaccatcgcggaacttgagccaatgacaaagataaacccaaggggagaaggcgaagcaatgccaagagaagacgctacccaactagaagtaactctaccatcttccattcatgagactggccctgataccatccaagaaccagactatgagccaaagcctactcaggagcctgccccagagcctgccccaggatcagagcctatggcagtgcctgaccttgacatcgagctggagctggagccagaaacgcagtcggaattggagccagagccagagccagagccagagtcagagcctggggttgtagttgagcccttaagtgaagatgaaaagggagtggttaaggcataggctggtggcctaagtacagcattaatcattaaggaacccattactgccatttggaattcaaataacctaaccaacctccacctcctccttccattttgaccaaccttcttctaacaaggtgctcattcctactatgaatccagaataaacacgccaagataacagctaaatcagcaagggttcctgtattaccaatatagaatactaacaattttactaacacgtaagcataacaaatgacagggcaagtgatttctaacttagttgagttttgcaacagtacctgtgttgttatttcagaaaatattatttctctctttttaactactctttttttttattttagacggagtcttgctccgtcgcgcaggctgtgatcgtagtggtgcgatctcggctcactgcagcctccgctccctgggttcaggagaatcgcttgaacccaggaggtggaggttgcagtgagccgagat

SEQ ID NO: 25 - DNA, 4028 bp, Homo sapiens
  misc_feature: Incyte ID No 70475510CB1
atgcctcctgtgtatgcctctgagtatgtcttgccactccagggtggagggtccggggaggagcaactctatgctgactttccagaacttgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattgataacagccttaaccctcgaggaatacctcaatacctcagacaaggcccttccaatatgtttacgttttcaaagaaatcaagtatatgaggagagcgagcgagcgtgagagaacacccgtgagagagacttgaaactgctgtcctttaaaaaaaaaaaaaatcaatgtttacattgaacaaagctgcttctgtctgtgagtttccatggtgttgacgttccactgccacattagtgtcctcgcttccaacgggttgtcccgggtgcacctcgaagtgccgggtccgtcacccatcgccccttccttcccgactgacttcctctcgtagacttgcagctgtgttcaccataacatttcttgtctgtagtgtgtgatgatgaaattgttacttgtgaatagaatcaggactataaacttcatttttaattgaaaaaaaaagtatatccttaaaataatgtatttatggctcagatgtactgtgcctgggattatgtattgcttccttgattttttaactatgcactgtcatgaggtgttgccactgagctgccctgctccccttgccagattgccctggaggtgctgggtggccgctaggctggtctgcaggaaagcgcggcctgccgtttccgggccgtatctgccaagccctgccttgtctcttactgagcaagttggctcaaattataggagcccccatcttgtgcccagctcatgctccaagtgtgtgtctatccattgttactcagactcttgagtaccttgtaagggaaggcggggcaaaggctgcatcaattcctgttttccagggggaggctggagtcctcaagagggcgaaatgactgtggaggtccggtacagtgaggaggaaagagggtgaccagaccgggctcggtctggccgggttccgataggggtaagcccggtccgacgagagggactcgctactggccggctaagccaggccataggggaccaagggtgcccccaacgggatctgccggcgttgggacccacatacacagcaggcggacaaggcgaatataaccgggaagggagacatgcgccacacagcacgaagaggcgagagcaaccaacatgcggcgacac

SEQ ID NO: 26 - DNA, 3320 bp, Homo sapiens
  misc_feature: Incyte ID No 566361CB1
ccgcccagccgctcgcaggcgccgcacggagttgcgtcccggggacttggggccgcagggagctgtgagtacccaggaagctgcaccgtgtggcctggagctgtctatctgtccttccagccacctgtctgtccagccacccttccacagactgaggcttgacaccggagcatctgtacagagcaaggagaagacaagaacatgctctaaagcccttcacagcaagacccaggaagccgcgggcaaactcagactcgaagccctcccgcctcctgcccacaatggcctctgctgacaagaatggcgggagcgtgtcctctgtgtccagcagccgcctgcagagccggaagccacccaacctctccatcaccatcccgccacccgagaaagagacccaggcccctggcgagcaggacagcatgctgcctgagaggaagaacccagcctacttgaagagcgtcagcctccaggagccacgcagccgatggcaggagagttcagagaagcgccctggcttccgccgccaggcctcactgtcccagagcatccgcaagggcgcagcccagtggtttggagtcagcggcgactgggaggggcagcggcagcagtggcagcgccgcagcctgcaccactgcagcatgcgctacggccgcctgaaggcctcgtgccagcgtgacctggagctccccagccaggaggcaccgtccttccagggcactgagtccccaaagccctgcaagatgcccaagattgtggatccgctggcccggggccgggccttccgccacccggaggagatggacaggccccacgccctgcacccaccgctgacccccggagtcctgtccctcacctccttcaccagtgtccgttctggctactcccacctgccacgccgcaagagaatgtctgtggcccacatgagcttgcaagctgccgctgccctcctcaaggggcgctcggtgctggatgccaccggacagcggtgccgggtggtcaagcgcagctttgccttcccgagcttcctggaggaggatgtggtcgatggggcagacacgtttgactcctccttttttagtaaggaagaaatgagctccatgcctgatgatgtctttgagtcccccccactctctgccagctacttccgagggatcccacactcagcctcccctgtctcccccgatggggtgcaaatccctctgaaggagtatggccgagccccagtccccgggccccggcgcggcaagcgcatcgcctccaaggtgaagcactttgcctttgatcggaagaagcggcactacggcctcggcgtggtgggcaactggctgaaccgcagctaccgccgcagcatcagcagcactgtgcagcggcagctggagagcttcgacagccaccggccctacttcacctactggctgaccttcgtccatgtcatcatcacgctgctggtgatttgcacgtatggcatcgcacccgtgggctttgcccagcacgtcaccacccagctggtgctgcggaacaaaggtgtgtacgagagcgtgaagtacatccagcaggagaacttctgggttggccccagctcgattgacctgatccacctgggggccaagttctcaccctgcatccggaaggacgggcagatcgagcagctggtgctgcgcgagcgagacctggagcgggactcaggctgctgtgtccagaatgaccactccggatgcatccagacccagcggaaggactgctcggagactttggccacttttgtcaagtggcaggatgacactgggccccccatggacaagtctgatctgggccagaagcggacttcgggggctgtctgccaccaggaccccaggacctgcgaggagccagcctccagcggtgcccacatctggcccgatgacatcactaagtggccgatctgcacagagcaggccaggagcaaccacacaggcttcctgcacatggactgcgagatcaagggccgcccctgctgcatcggcaccaagggcagctgtgagatcaccacccgggaatactgtgagttcatgcacggctatttccatgaggaagcaacactctgctcccaggtgcactgcttggacaaggtgtgtgggctgctgcccttcctcaaccctgaggtcccagatcagttctacaggctctggctgtctctcttcctacatgctggcgtggtgcactgcctcgtgtctgtggtctttcaaatgaccatcctgagggacctggagaagctggccggctggcaccgtatcgccatcatcttcatcctcagtggcatcacaggcaacctcgccagtgccatctttctcccataccgggcagaggtgggcccggccggctcacagttcggcctcctcgcctgcctcttcgtggagctcttccagagctggccgctgctggagaggccctggaaggccttcctcaacctctcggccatcgtgctcttcctgttcatctgtggcctcctgccctggatcgacaacatcgcccacatcttcggcttcctcagtggcctgctgctggccttcgccttcctgccctacatcaccttcggcaccagcgacaagtaccgcaagcgggcactcatcctggtgtcactgctggcctttgccggcctcttcgccgccctcgtgctgtggctgtacatctaccccattaactggccctggatcgagcacctcacctgcttccccttcaccagccgcttctgcgagaagtatgagctggaccaggtgctgcactgaccgctgggccacacggctgcccctcagccctgctggaacagggtctgcctgcgagggctgccctctgcagagcgctctctgtgtgccagagagccagagacccaagacagggcccgggctctggacctgggtgcccccctgccaggcgaggctgactccgcgtgagatggttggttaaggcggggtttttctggggcgtgaggcctgtgagatcctgacccaagctcaggcacacccaaggcacctgcctctctgagtcttgggtctcagttcctaatatcccgctccttgctgagaccatctcctggggcagggtccttttcttcccaggtcctcagcgctgcctctgctggtgccttctcccccactactactggagcgtgcccttgctggggacgtggctgtgccctcagttgcccccagggctgggtgcccaccatgccccttcctctttctcctcctacctctgccctgtgagcccatccataaggctctcagatgggacattgtgggaaaggctttggccatggtctgggggcagagaacaagggggga

SEQ ID NO: 27 - DNA, 2914 bp, Homo sapiens
  misc_feature: Incyte ID No 71969340CB1
ctccctccccgcgcttacgtcgcgcggccatgcggtttggacaggacacccctgagagtgcaggcacctccccctcccgcccctccatccctctgggggctggcgcctggccccccacctggtccccctgggcaggctgaattggggctccctgcagggcggtcccgatggccgggcgtgggtggggcgcgctgtgggtgtgcgtggcggccgccaccctgctgcacgctggcggcctggcccgcgcagactgctggctgatcgagggcgacaagggcttcgtgtggctggccatctgcagccagaaccaacccccctacgaggccatcccacagcagatcaacagcaccatcgtggacctgcggctcaacgagaaccgtatccgcagcgtgcagtacgcctcgctcagccgctttggcaacctcacgtacctcaacctcaccaagaacgagatcggctacatcgaggacggcgccttctcgggccagttcaacctgcaggtgctgcagctgggctacaaccggctgcgcaacctcacggagggcatgctgcgcggcctgggcaagctggagtacctgtacctgcaggccaacctcatcgaggtggtcatggccagcagcttctgggagtgtcccaacatcgtcaacatcgacctgtccatgaaccgcatccagcagctcaacagcggcaccttcgccggcctggccaagctgtcggtgtgcgagctctacagcaaccccttctactgctcctgcgagctgctgggcttcctgcgctggctggccgccttcaccaacgccacacagacgtacgaccgcatgcagtgcgagtcgccgcccgtctactccggctactacctcctgggccagggccgccgcggccaccgcagcatcctcagcaaactgcagtcagtctgcaccgaggactcgtacgcggctgaggtggtcgggcccccacgtccagcatccgggcgctcacagccgggccgctccccgccgcccccgcctccgccggagcccagtgacatgccctgtgccgatgatgagtgcttctccggggacggcaccacgccactggtggccctgcccacgctggccacgcaggccgaggcccgccccctcatcaaggtcaagcagctcactcagaactcggccaccatcaccgtccagctgcccagcccgttccaccggatgtacaccctggagcatttcaacaacagcaaggcctccaccgtgtccaggctgaccaaggcccaggaggagatccgtctgaccaacctgttcacgctcaccaactacacctactgcgtggtgtccaccagcgccgggctgcgccacaaccacacctgcctcaccatctgcttgccccggctgcccagcccgcctggtccggtgcccagcccctccacggccacccactacatcatgaccatcctgggctgcctcttcggcatggtgctggtgctgggcgccgtctactactgcctgcgcaggcggcggcgccaggaggagaagcacaagaaggccgcctcggcagccgcagctggcagcctcaagaagaccatcatcgagctcaagtacgggccagagctggaggcgcccggcctggccccgctgtcccagggcccgctgctgggccccgaggccgtgacgcgcatcccttacctgcctgcggccggcgaggtggagcagtacaagctggtggagagcgcggacacccccaaggccagcaagggcagctacatggaggttcgaaccggggaccctccggaacgcagggactgtgagctgggccggccgggccccgacagccagagttcggtggccgagatctccaccatcgccaaggaggtggacaaggtcaaccagatcatcaacaactgcatcgacgcgctcaagtccgagtccacctccttccagggcgtcaagtcggggcccgtgtccgtcgcggagccgccgctggtgctgctgtccgagccgctggccgccaagcacggcttcctggcgcccgggtacaaggacgccttcggccacagcctgcagcggcaccacagcgtggaggccgccgggccccctcgtgccagcacctcgtccagcggctccgtgcgcagcccccgcgccttccgagccgaggccgtcggggtgcacaaggccgcggccgccgaggccaagtacatcgagaagggctcccccgcggccgacgccatcctcactgtgacacccgcggccgccgtgctgcgggccgaggccgagaagggtcgccagtacggcgagcaccggcactcgtaccccggctcccacccggccgagccacctgcgccccccgggccaccgccgccgcctccgcacgagggcctggggcgcaaggcgtccatcctggagccactcacccggccgcggccccgcgacctcgcctactcgcagctgtccccgcagtaccacagcctgagctactcctccagccccgagtacacctgccgggcctcccagagcatctgggagcgcttcagactgagccgccggcggcacaaggaggaagaggagttcatggccgcgggccatgccctgcgcaagaaggttcagttcgccaaagacgaggatctgcacgacatcctggactactggaagggcgtgtcggcccagcacaagtcctgagccccccaagaccggcgatgcccactggaccaaaaggatgcaggatccacccagagactcagcaccaaacccaacacacgcacgccaccacagcaactgtgacagcggggggccctgcagaggcgaggggggagcgagtggggacagacaagggggacacgtcccgagctcctgtggccggtcctgggatgcgcttgtcgccccgggtggcacgtgtccacacacacacacagacacacacacacacacacacacacgcg

SEQ ID NO: 28 - DNA, 3990 bp, Homo sapiens
  misc_feature: Incyte ID No 6772808CB1
cacctgcccggcgccgcctccgcccgcccccaccgcggcgcaacttggatggagttggggtcctgagcgccggccccccacagccgccagcgcagagctcgtgccgccaccttcgttctgggacccctctctccgctgctcttcgctcccgcgatgggaaaagttggcgccggcggcggctcccaagcccggctgagcgcgctcctcgccggcgcggggctcttgatcctctgcgccccgggcgtctgcggcggcggctcctgctgcccctcgccgcaccccagctcggctccacgctcggcctcgacccctaggggcttttcccaccaggggcggccaggcagggctcctgccacgcccctgcccctcgtagtgcgtcccctgttctcagtggcccccggggaccgagcgctatccctggagcgggctcggggcactggggcatccatggcggttgctgcacgctccggccggaggagacggagcggagcggatcaggagaaggcagaacggggagagggcgcgagtcggagcccccggggagtgctaagagatggagggcagcaggagcctgggactcgggagcgggacccggacaaagccacccgcttccggatggaggagctgagactgaccagcaccacgtttgcgctgacgggagactcagcacacaaccaagccatggtccactggtctggccacaacagcagcgtgattctcattttgacaaagctctatgactataacctggggagcatcacagagagctcgctttggaggtcaaccgattatggaacaacctatgagaagctgaatgataaagttggtttgaaaaccattttgagctatctctatgtgtgtcctaccaacaagcgtaagataatgttactcacagacccggagattgagagcagtttattgatcagctcagatgaaggggcaacttatcaaaagtaccggctgaacttctacattcaaagcttgctttttcaccccaaacaagaagactggattctggcatacagtcaagaccaaaagttatacagctctgctgaatttgggagaagatggcagcttatccaagaaggggttgtaccaaacaggttctactggtctgtgatggggtcaaataaagaaccagaccttgtgcatcttgaggccagaactgtggatggtcattcacattatctaacttgccgaatgcagaactgtacagaggccaacaggaatcagccttttccaggctacattgacccagactctttgattgttcaggatcattatgtgtttgttcagctgacatcaggagggcggccacattactacgtgtcctaccgaaggaatgcatttgcccaaatgaagcttccgaaatatgctttgcccaaggacatgcatgttatcagcaccgatgagaatcaggtgttcgcagcggtccaagaatggaaccagaatgacacgtacaacctctacatctcagacacacgtggtgtctacttcaccctggccttggagaatgtccagagcagcagaggccctgagggcaacatcatgatcgacctctatgaggtagcagggataaagggaatgttcttggctaacaagaagattgacaaccaagtgaagactttcatcacatataacaaaggcagagactggcgtttgctgcaggcgccggacacggatctaaggggggaccccgtgcactgcttgctgccctattgctcactacaccttcacctgaaggtctctgagaatccctacacatcagggatcattgccagcaaagacacagctccaagcatcatagtggcatcaggtaatataggttctgaattgtcagacactgacatcagcatgtttgtctcttcagatgcagggaacacctggagacagatctttgaagaagagcacagtgttttgtacctggatcaaggtggagtcctggttgctatgaaacacacatctctcccaattcgacatctttggttgagttttgatgaagggagatcttggagcaaatacagtttcacatctattccactttttgtggatggggttctgggtgagcctggagaagagactctcatcatgacagtgtttggacacttcagccaccgctctgaatggcagctggtcaaagtagattacaagtccatttttgatagacggtgtgccgaagaggactacagaccttggcagctgcacagccagggggaagcatgtatcatgggagcaaaaaggatatataagaagcgaaaatcagagcggaagtgtatgcaaggaaaatatgcaggagctatggaatctgaaccctgtgtctgcactgaggctgattttgattgcgactatggttatgagcgacacagcaatggccagtgcctgccggcattttggttcaatccatcctctctgtcaaaggattgcagcttgggacagagttacctcaatagtactgggtacaggaaggtggtttccaataattgcactgatggcgtaagggaacagtacactgccaaaccgcagaagtgcccagggaaagccccgcgggggctgcggatagtcacggctgatggaaagctgacagcggaacaaggacacaacgtcactctcatggtgcaattagaagagggtgatgttcagcggacgctcatccaagtggactttggcgatggtatcgcggtgtcttacgtcaatctcagctccatggaagatgggatcaaacacgcctatcagaacgtgggcattttccgtgtgaccgtgcaggtggacaacagtctgggttctgacagcgccgtcctgtacttacatgtaacttgtcccttggagcacgtgcacctgtctcttccctttgtcaccacaaagaacaaagaggtcaatgcgacggcagtgctgtggcccagccaagtgggcaccctcacttacgtgtggtggtacggaaacaacacggagcctttgatcaccttggagggaagcatatccttcagatttacttcagaaggaatgaataccatcacagtgcaggtctcagctgggaatgccatcctacaagacacaaagaccatcgcagtatatgaggaattccggtctcttcgcttgtccttttctccaaacctggatgactacaacccggacatccctgagtggaggagggacatcggtcgagtcatcaaaaaatccctggtggaagccacaggggttccaggccagcacatcctggtggcggtgctccctggcttacccaccactgctgaactctttgtcctaccctatcaggatccagctggagaaaacaaaaggtcaactgatgacctggagcagatatcagaattgctgatccacacgctcaaccaaaactcagtacacttcgagctgaagccaggagtccgagtccttgtccatgctgctcacttaacagcggcccccctggtggacctcactccaacccacagtggatctgccatgctgatgctgctctcagtggtgtttgtggggctggcagtgttcgtcatctacaagtttaaaaggagagtagctttaccctcccctccctccccttctactcaacctggtgactcatctctccgattgcaaagagcaagacacgccactccgccttcaacgccaaagcggggatctgctggggcacagtatgcaatttaaggaaaacccccaaaggctacaggcgacctgctgatcaggaaagaatttcgctcttgtcaagtacatcatccttcatgaccactaactttgtgtttttttttctttcctttgttggttcctgtttccctaattttggccagcgaangtactttccantcnagttgctggagaatcacaagcacannaaagaaatccctaccttatgtaaactgctttgacactggcaggacgcccagtacacaaaaacaaaaacaaaaacaaaacaaaacataaaatataaacaatcaaaatccaaacaaacaaacaaacactcactgcatcgggactttt

SEQ ID NO: 29 - DNA, 1198 bp, Homo sapiens
  misc_feature: Incyte ID No 60137669CB1
gcggcgatggcccagcccggggaccctgcggcgcctctgcaggctggtgcaggagggccggctgcgcgccctgaaggaggagctgcagtgctggccggtggtgctgccctggtggcctggccggggataccctcctgcactgcgccgcgcgccacgggcatcgggacgtgctggcctatctggccgaggcctggggcatggacatcgaggccaccaaccgagactacaagcggcctctgcacgaggcggcctccatgggccaccgagactgcgtgcgctacctgctgggccggggggcagcggtcgactgcctgaagaaggccgactggactcctctgatgatggcctgcacaaggaagaacctgggggtgatccaggagctggtggaacatggcgccaatccactcctgaagaacaaagatggctggaacagtttccacattgccagtcgagaaggcgaccctctgatcctccagtacctgctcactgtttgcccaggtgcctggaagacagagagcaaaattagaaggactcctctgcatactgcagcaatgcatggccatttggaggcagtcaaggtgcttcttaagaggtgccaatatgaaccagactacagagacaactgtggcgtcaccgccttgatggacgcaatccagtgtggtcacatcgacgtcgctaggctgctcctcgatgaacatggggcttgcctttcagcagaagacagcctgggtgcccaggctctgcacagggcagctgtcacagggcaggacgaagccatccgattcttggtctctgaacttggcgtcgatgtagatgtgagagccacatcaacccacctcacagcacttcattatgcagctaaggaaggacatacaagtacaattcagactctcttatccttgggagctgacatcaattctaaagatgaaaaaaatcgatcagccctgcatctggcctgtgcaggtcagcacttggcctgtgccaagtttctcctgcagtcgggactgaaggattctgaagacatcacgggcaccctggctcagcagctcccaaggagagcagatgtccttcggggctctggccatagcgcaatgacataaggatgtttccaagaggaggcaataaagtgcatggtaattccaaaaaaaaaaaaaaaaaactctttgtcgggtgcggaaaaagcaggtattgaattggc

SEQ ID NO: 30 - DNA, 1297 bp, Homo sapiens
  misc_feature: Incyte ID No 1987928CB1
gctctgcaagtggtgaccccgacgtgatcgccttgaagttacgcttgaaggaggaaaactcatcaattttcggggaatcccgcctttgtttcccaggctctctgagcacgatgtctgcagctcccgccagcaatggagtgtttgttgtcatcccgccaaacaacgccagtggcctctgcccacctccggccattctgcccacatccatgtgccaacctccagggattatgcagtttgaggagccaccgctgggggcacagacaccaagggccacacagccacctgacttgcggcccgtggagacattcctgacaggagagcccaaagttttggggacggtgcagatcctcatcggcctcatccacctaggctttggcagcgtgctgctcatggttcgccgcggccacgtgggcatcttcttcatcgagggcggcgtccccttctggggaggagcctgcttcatcatctccggatccctctcagtggcagccgagaagaaccacaccagttgcctggtgaggagcagcctgggcaccaacatcctcagcgtcatggcggcctttgctgggacagccattctgctcatggattttggtgttaccaaccgggatgtggacaggggctatctggccgtgcttactatcttcactgtcctggagttcttcacagcggtcattgccatgcacttcgggtgccaagccatccatgcccaggccagtgcacctgtgatcttcctgccaaacgccttcagcgcagacttcaacatccccagcccggcagcctctgcgccccctgcctatgacaatgtggcatatgcccaaggagtcgtctgagtagcagatgtggcacctgcgggtggagtccagccttttccctctgggcccagcctctccccacccccaccttgttcatcaggggccagccccatcccagctgccctccctcaccacatctacacatactccggcatctgagtgaagtgtccccagggacatctctcccacactttccgcagtgctttctttctaaaagacaccgggctgacgtcaggggtgtgtgtccttcagctccctgagccctgtcacccttccaggacacccaccttgtgcatctaagcatttctctgctcattggggaaatcctggcctcattggagactcaggttcgaggcctgccctgaccctcgggcctcgggaaggtcagagagcccggaatcctccagaatggaagagtctgactctggcattccacagaggtgccgataccaggccaaggcctcacagcagggtagtggcct

SEQ ID NO: 31 - DNA, 2482 bp, Homo sapiens
  misc_feature: Incyte ID No 7268131CB1
gatgagcacacgggagaggagaagagggagacccgccgcctccctccctccctagctgacttgctccctcccgggctgcggctgctgcaaaagccagcagcggcagcgggagctgtccggaggccggcgtcgagggtttgccgctgtctctgctattccatcctccccataggggctctctcccctctcccatctcaagatggcagccagcagctctgagatctctgagatgaagggggttgaggagagtcccaaggttccaggcgaagggcctggccattctgaagctgaaactggccctccccaggtcctagcaggggtaccagaccagccagaggccccgcagccaggtccaaacaccactgcggcccctgtggactcagggcccaaggctgggctggctccagaaaccacagagaccccggctggggcctcagaaacagcccaggccacagacctcagcttaagcccaggaggggaatcaaaggccaactgcagccccgaagacccatgccaagaaacagtgtccaaaccagaagtgagcaaagaggccactgcagaccaggggtccaggctggagtctgcagccccacctgaaccagccccagagcctgctccccaaccagacccccggccagattcccagcctacccccaagccagcccttcaaccagagctccctacccaggaggaccccacccctgagattctgtctgagagtgtaggggaaaagcaagagaatggggcagtggtgcccctgcaggctggtgatggggaagagggcccagcccctgagcctcactcaccaccctcaaaaaaatcccccccagccaatggggcccccccccgagtgctgcagcagctggttgaggaggatcgaatgagaagggcacacagtgggcatccaggatctccccgaggtagcctgagccgccaccccagctcccagctggcaggtcctggggtggaggggggtgaaggcacccagaaacctcgggactacatcatccttgccatcctgtcctgcttctgccccatgtggcctgtcaacatcgtggccttcgcttatgctgtcatgtcccggaacagcctgcagcagggggacgtggacggggcccagcgtctgggccgggtagccaagctcttaagcatcgtggcgctggtggggggagtcctcatcatcatcgcctcctgcgtcatcaacttaggcggtgagtgggggcttgggacaggcaggggaggaatggaagggttggcaagggcagctttactaacccctgcccctgctctctcctgtctgtcctccttacctctcctttgtctctccttgtctccccctccccccgtctgtccttccctctcctctcccacagtgtataagtgaggggctctgccccgcatcccaagacttttcttcctgttgggagctgccttgggcccatccctcccctggggggagcccaactgatggccctggcccccacccctaaggaccaagggagcctgagcggccttgtttacagcttctgtcctgctcctgcatcttgccaggctcctctgccaactgtaggcctgcctcatccctgcactggttccaacctccctgcactaatgcctgcatcccctccggcctcttggccccctatccctgcacttctggaaacctccctgcactctggaaacctccctgaacacctccccaactctgcgctctcagcctccctgcatctctcctggcctccctgcacttcttccagccccccaaattctctggacctccaccctggccgcctcctcccaactttcattgtcttggcatctctcaaccctcagtcctctcttccttcccttctttatcatctcccctttcctctccacgtcccgcccccttcctcttcctgcctcctcatctcccttaagcatcctcttctccaacctcccgtcaccgtttactctgcaaaactgacagcacttagacgaggcttgggggcagggagcagtgttgggagagggctccccaaccccaggctcggactgttctctgctgggaccacccagggtcggacacccaagggtgcctggcaggtcgcagagttggcaagccgggcctcgtatggggactcgggtgagggtggcgagtactggttccgaacgcacgcaggggagaagggagggacgcggcgctgacccttccaggtcagctggagttgacccgcccacctgggcttttcaaccccagtccgcgagtttctttcttgaaggtgtgggggctagattcattcacgtgcttcgtaatgaaataatccaaaaaataggaccaaagcgcccactggcaggagcgagggcggggcgccgcgctctataattattttctaagatgatgggggaggtttgttgcacgcgacagcccgctgaggaggcggggaccgagctacaacgcggttcggatttggcgggggtttttttccttaaaaaaaaaaaa

SEQ ID NO: 32 - DNA, 2323 bp, Homo sapiens
  misc_feature: Incyte ID No 7285339CB1
gaggggatgagcacacgggagaggagaagagggagacccgccgcctccctccctccctagctgacttgctccctcccgggctgcggctgctgcaaaagccagcagcggcagcgggagctgtccggaggccggcgtcgagggtttgccgctgtctctgctattccatcctccccataggggctctctcccctctcccatctcaagatggcagccagcagctctgagatctctgagatgaagggggttgaggagagtcccaaggttccaggcgaagggcctggccattctgaagctgaaactggccctccccaggtcctagcaggggtaccagaccagccagaggccccgcagccaggtccaaacaccactgcggcccctgtggactcagggcccaaggctgggctggctccagaaaccacagagaccccggctggggcctcagaaacagcccaggccacagacctcagcttaagcccaggaggggaatcaaaggccaactgcagccccgaagacccatgccaagaaacagtgtccaaaccagaagtgagcaaagaggccactgcagaccaggggtccaggctggagtctgcagccccacctgaaccagccccagagcctgctccccaaccagacccccggccagattcccagcctacccccaagccagcccttcaaccagagctccctacccaggaggaccccacccctgagattctgtctgagagtgtaggggaaaagcaagagaatggggcagtggtgcccctgcaggctggtgatggggaagagggcccagcccctgagcctcactcaccaccctcaaaaaaatcccccccagccaatggggcccccccccgagtgctgcagcagctggttgaggaggatcgaatgagaagggcacacagtgggcatccaggatctccccgaggtagcctgagccgccaccccagctcccagctggcaggtcctggggtggaggggggtgaaggcacccagaaacctcgggactacatcatccttgccatcctgtcctgcttctgccccatgtggcctgtcaacatcgtggccttcgcttatgctgtcatgtcccggaacagcctgcagcagggggacgtggacggggcccagcgtctgggccgggtagccaagctcttaagcatcgtggcgctggtggggggagtcctcatcatcatcgcctcctgcgtcatcaacttaggcgtgtataagtgaggggctctgccccgcatcccaagacttttcttcctgttgggagctgccttgggcccatccctcccctggggggagcccaactgatggccctggcccccacccctaaggaccaagggagcctgagcggccttgtttacagcttctgtcctgctcctgcatcttgccaggctcctctgccaactgtaggcctgcctcatccctgcactggttccaacctccctgcactaatgcctgcatcccctccggcctcttggccccctatccctgcacttctggaaacctccctgcactctggaaacctccctgaacacctccccaactctgcgctctcagcctccctgcatctctcctggcctccctgcacttcttccagccccccaaattctctggacctccaccctggccgcctcctcccaactttcattgtcttggcatctctcaaccctcagtcctctcttccttcccttctttatcatctcccctttcctctccacgtcccgcccccttcctcttcctgcctcctcatctcccttaagcatcctcttctccaacctcccgtcaccgtttactctgcaaaactgacagcacttagacgaggcttgggggcagggagcagtgttgggagagggctccccaaccccaggctcggactgttctctgctgggaccacccagggtcggacacccaagggtgcctggcaggtcgcagagttggcaagccgggcctcgtatggggactcgggtgagggtggcgagtactggttccgaacgcacgcaggggagaagggagggacgcggcgctgacccttccaggtcagctggagttgacccgcccacctgggcttttcaaccccagtccgcgagtttctttcttgaaggtgtgggggctagattcattcacgtgcttcgtaatgaaataatccaaaaaataggaccaaagcgcccactggcaggagcgagggcggggcgccgcgctctataattattttctaagatgatgggggaggtttgttgcacgcgacagcccgctgaggaggcggggaccgagctacaacgcggttcggatttggcgggggtttttttccttaaaaaaaaaa

SEQ ID NO: 33 - DNA, 2232 bp, Homo sapiens
  misc_feature: Incyte ID No 7495197CB1
gcgagggcgcaggtggaaagcgggagagcgcggatgatacctagtggggccagaggagtcttcctcttctaggggctcccggagctcggcgggcccctgttcgcagtacaggaggtagcagaaggcacacctgaagccagcgctggagggaagggcgagggtcagcttccacccctttccgccctggagaccgctgatgtcgctttatggttgtagcaagtttaatcatcctccatttgtctggggcaaccaagaaaggaacagaaaagcaaaccacctcagaaacacagaagtcagtgcagtgtggaacttggacaaaacatgcagagggaggtatctttacctctcccaactatcccagcaagtatccccctgaccgggaatgcatctacatcatagaagccgctccaagacagtgcattgaactttactttgatgaaaagtactctattgaaccgtcttgggagtgcaaatttgatcatattgaagttcgagatggaccttttggcttttctccaataattggacgtttctgtggacaacaaaatccacctgtcataaaatccagtggaagatttctatggattaaattttttgctgatggagagctggaatctatgggattttcagctcgatacaatttcacacctgatcctgactttaaggaccttggagctttgaaaccattaccagcgtgtgagtttgagatgggcggttccgaaggaattgtggagtctatacaaattatgaaggaaggcaaagctactgctagcgaggctgttgattgcaagtggtacatccgagcacctccacggtccaagatttacttacgattcttggactatgagatgcagaattcaaatgagtgcaagaggaattttgtggctgtgtatgatggaagcagttccgtggaggatttgaaagctaagttctgtagcactgtggctaatgatgtcatgctacgcacgggtcttggggtgatccgcatgtgggcagatgagggcagtcgaaacagccgatttcagatgctcttcacatcctttcaagaacctccttgtgaaggcaacacattcttctgccatagtaacatgtgtattaataatactttggtctgcaatggactccagaactgtgtgtatccttgggatgaaaatcactgtaaagagaagaggaaaaccagcctgctggaccagctgaccaacaccagtgggactgtcattggcgtgacttcctgcatcgtgatcatcctcattatcatctctgtcatcgtacagatcaaacagcctcgtaaaaagtatgtccaaaggaaatcagactttgaccagacagttttccaggaggtatttgaacctcctcattatgagttatgcactctcagagggacaggagctacagctgactttgcagatgtggcagatgactttgaaaattaccataaactgcggaggtcatcttccaaatgcattcatgaccatcactgtggatcacagctgtccagcactaaaggcagccgcagtaacctcagcacaagagatgcttctatcttgacagagatgcccacacagccaggaaaacccctcatcccacccatgaacagaagaaatatccttgtcatgaaacacaactactcgcaagatgctgcagatgcctgtgacatagatgaaatcgaagaggtgccgaccaccagtcacaggctgtccagacacgataaagccgtccagcggttctgcctcattgggtctctaagcaaacatgaatctgaatacaacacaactagggtctagaaagaaaattcaagacagcttgagaatagtgcgttcctgaatgattttgaacatgctacagtgaaaagtgacagtgtggaccatggaatcaccagctagagatgaggaaactgaagagttttagtaacttttttaagattacacaataaacaatgatgaatcaagctttgaagccaacctcaccaaccacaagatcaaccaacactcttcaccaatgtgtaatataaccacgttaatattcaacatagtacgtactgctgaaagaagttgatacttattcatattaaccccgtagttttgtgtttcctcatctgtaaaagtatgtattataacaccttctctccaccttacagcgtgtgaggttcaaatgaccattcattggaagatattttttatatcctataatgcattataaaaataaatcatttttcctaaaaaaaaaaaaaaa

SEQ ID NO: 34 - DNA, 7590 bp, Homo sapiens
  misc_feature: Incyte ID No 3954126CB1
gattgcacgagtcggatccctgggacgcagcttccactcctgttctaactatttgtgattgaaaaaaggaaacgagactaggaacacaattgcaagtggtgttcctaaaaggaaaacacatacgctccaaaaggaggggaagaacaacccagttggcgtgcacattttttttaaaggagaattcctcagacactacatggagttatgtggaaatgagagagattcatgaaacccctcctccaggaaagaatgtctttcacagatggagcttgcttctggtttgcacaggacagcgacaatgtggcagagccatgcctgcccttcctgctctgtccagtgattcacagaacttctgaacagtgatgcttgccttggattttcaggttttcatcctgatacttgtttacttttctggggcagaaaagcttgcactaattgctctccatggtggctaattttttcaagagcttgattttaccttacattcataagctttgcaaaggaatgtttacaaagaaattgggaaatacaaacaaaaacagagagtatcgtcagcagaaaaaggatcaagacttccccactgctggccagaccaaatcccccaaattttcttacacttttaaaagcactgtaaagaagattgcaaagtgttcatccactcacaacttatccactgaggaagacgaggccagtaaagagttttccctctcaccaacattcagttaccgagtagctattgccaatggcctacaaaagaatgctaaagtaaccaacagtgataatgaggatctgcttcaagagctctcttcaatcgagagttcctactcagaatcattaaatgaactaaggagtagcacagaaaaccaggcacaatcaacacacacaatgccagttagacgcaacagaaagagttcaagcagccttgcaccctctgagggcagctctgacggggagcgtactctacatggcttaaaactgggagctttacgaaaactgagaaaatggaaaaagagtcaagaatgtgtctcctcagactcagagttaagcaccatgaaaaaatcctggggaataagaagtaagtctttggacagaactgtccgaaacccaaagacaaatgccctggagccagggttcagttcctctggctgcattagccaaacacatgatgtcatggaaatgatctttaaggaacttcagggaataagtcagattgaaacagaactttctgaactacgagggcacgtcaatgctctcaagcactccatcgatgagatctccagcagtgtggaggttgtacaaagtgaaattgagcagttgcgcacagggtttgtccagtctcggagggaaactagagacatccatgattatattaagcacttaggtcatatgggtagcaaggcaagcctgagatttttaaatgtgactgaagaaagatttgaatatgttgaaagcgtggtgtaccaaattctaatagataaaatgggtttttcagatgcaccaaatgctattaaaattgaatttgctcagaggataggacaccagagagactgcccaaatgcaaagcctcgacccatacttgtgtactttgaaacccctcaacaaagggattctgtcttaaaaaagtcatataaactcaaaggaacaggcattggaatctcaacagatattctaactcatgacatcagagaaagaaaagagaaagggataccatcctcccagacatatgagagcatggctataaagttgtctactccagagccaaaaatcaagaagaacaattggcagtcacctgatgacagtgatgaagatcttgaatctgacctcaatagaaacagttacgctgtgctttccaagtcagagcttctaacaaagggaagtacttccaagccaagctcaaaatcacacagtgctagatccaagaataaaactgctaatagcagcagaatttcaaataaatcagattatgataaaatctcctcacagttgccagaatcagatatcttggaaaagcaaaccacaacccattatgcagatgcaacacctctctggcactcacagagtgattttttcactgctaaacttagtcgttctgaatcagatttttccaaattgtgtcagtcttactcagaagatttttcagaaaatcagtttttcactagaactaatggaagctctctcctgtcatcttcggaccgggagctatggcagaggaaacaggaaggaacagcgaccctgtatgacagtcccaaggaccagcatttgaatggaggtgttcagggtatccaagggcagactgaaactgaaaacacagaaactgtggatagtggaatgagtaatggcatggtgtgtgcatctggagaccggagtcattacagtgattctcagctctctttacatgaggatctttctccatggaaggaatggaatcaaggagctgatttaggcttggattcatccacccaggaaggttttgattatgaaacaaacagtctttttgaccaacagcttgatgtttacaataaagacctagaatacttgggaaagtgccacagtgatcttcaagatgactcagagagctacgacttaactcaagatgacaattcttctccatgccctggcttggataatgaaccacaaggccagtgggttggccaatatgattcttatcagggagctaattctaatgagctataccaaaatcaaaaccagttgtccatgatgtatcgaagtcaaagtgaattgcaaagtgatgattcagaggatgccccacccaaatcatggcatagtcgattaagcattgacctttctgataagactttcagcttcccaaaatttggatctacactgcagagggctaaatcagccttggaagtagtatggaacaaaagcacacagagtctgagtgggtatgaggacagtggctcttcattaatggggagatttcggacattatctcaatcaactgcaaatgagtcaagtaccacacttgactctgatgtctacacggagccctattactataaagcagaggatgaggaagattatactgaaccagtggctgacaatgaaacagattatgttgaagtcatggaacaagtccttgctaaactagaaaacaggactagtattactgaaacagatgaacaaatgcaagcatatgatcacctttcatatgaaacaccttatgaaaccccacaagatgagggttatgatggtccagcagatgatatggttagtgaagaggggttagaacccttaaatgaaacatcagctgagatggaaataagagaagatgaaaaccaaaacattcctgaacagccagtggagatcacaaagccaaagagaattcgtccttctttcaaagaagcagctttaagggcctataaaaagcaaatggcagagttggaagagaagatcttggctggagatagcagttctgtggatgaaaaggctcgaatagtaagtggcaatgatttggatgcttccaaattttctgcactccaggtgtgtggtggggctggaggtggactttatggtattgacagcatgccggatcttcgcagaaaaaaaactttgcctattgtccgagatgtggccatgaccctggctgcccggaaatctggactctccctggctatggtgattaggacatccctaaataatgaggaactgaaaatgcacgtcttcaagaagaccttgcaggcactgatctaccctatgtcttctaccatcccacacaattttgaggtctggacggctaccacacccacctactgttatgagtgtgaagggctcctgtggggcattgcaaggcaaggcatgaagtgtctggagtgtggagtgaaatgccacgaaaagtgtcaggacctgctaaacgctgactgcttgcagagagcagcagaaaagagttctaaacatggtgccgaagacaagactcagaccattattacagcaatgaaagaaagaatgaagatcagggagaaaaaccggccagaagtatttgaagtaatccaggaaatgtttcagatttctaaagaagattttgtgcagtttacaaaggcggccaaacagagtgtactggatgggacatctaagtggtctgcaaaaataaccattacagtggtttctgcacagggtctacaggcaaaagataaaacagggtctagtgatccatatgttacagttcaagttggaaagaacaaaagaagaacaaaaaccatttttggaaatttgaatccagtatgggatgagaagttttattttgagtgtcataactccacagatcgaatcaaagtcagagtatgggatgaagatgatgatattaaatccagagtcaagcaacatttcaaaaaggagtcagatgattttctgggacaaacaattgtagaagtgaggaccttgagtggagaaatggatgtctggtacaacttagagaaaaggacagataagtcagctgtatctggggccatacgattgaaaatcaatgtggagataaaaggagaagagaaggttgctccatatcatattcaatatacatgtttacatgagaatctgttccattacttgactgaagtgaaatctaatggtggagtgaaaatcccagaagtcaaaggggatgaagcctggaaggttttctttgatgatgcttcccaagaaatagttgatgaatttgctatgcgttatggaattgaatccatttatcaagctatgacgcacttttcatgtctgtcttctaaatacatgtgccccggtgtccctgccgtcatgagcaccttgctggctaatataaatgctttttatgctcacacaacagtttcaacaaacatacaggtttctgcctcagatcgatttgctgctaccaactttggtagggaaaaattcataaaactactggaccagttacataactctttgaggattgatctgtcaaagtatagggaaaactttcctgcaagcaatactgaaagactgcaagacctgaaatcaactgttgacctgttaacaagtatcaccttttttaggatgaaggttctggagctgcaaagccccccaaaagcgagcatggtggtgaaggactgtgtaagggcttgcctggattctacatacaagtatatttttgacaactgccatgaactctactcccagctaacagacccgagtaagaaacaggatattcctcgtgaagatcagggaccaaccaccaagaatttggatttttggccccaacttattacactgatggttactattattgatgaggataaaactgcctacacacctgtcctgaatcagtttcctcaagagctgaacatgggaaaaataagtgccgaaattatgtggactctttttgctctggatatgaaatatgcattagaagaacatgataatcagcggttatgcaagagcaccgattatatgaatttgcatttcaaagttaaatggttttataatgaatatgtgcgtgaacttcctgccttcaaggatgctgttcctgaatactccttgtggtttgaaccttttgtcatgcaatggctagatgaaaacgaagatgtgtcaatggaattccttcatggagcactgggaagagacaaaaaagatggattccagcagacatctgagcatgctctcttttcttgctccgtggttgatgtctttgctcagctgaatcagagctttgaaattattaagaaactggaatgccctaatcctgaagcattatctcacttaatgagaagatttgcaaagactatcaataaagtgctgctccagtatgctgcaattgtatcaagtgatttcagttcacattgtgataaggaaaatgtgccctgtatcttgatgaacaatattcaacaattgcgggtccagctggaaaaaatgtttgaatccatgggagggaaggagctagattctgaagctagtactattctaaaagaacttcaggttaagctcagtggggtcctggatgagctcagcgtcacttatggtgaaagtttccaggttataattgaagagtgtataaaacagatgagtttcgaactaaatcaaatgagagcaaatggaaacaccacatctaataagaacagtgcagcaatggatgcagagattgtgttaagatctcttatggattttttggacaaaacattaagtctctcagcaaaaatctgtgagaaaacagtcctaaagcgagttttaaaagagttatggaagctagttctcaacaaaatagaaaaacaaattgttcttcctcctctgacagatcaaacaggaccccagatgattttcattgcagctaaagatcttggacaattatccaaactgaaggagcacatgattcgagaggatgccaggggtctgacgccaagacaatgtgctataatggaggtagtcctggctaccatcaagcaatactttcatgcaggaggaaatggcctgaaaaagaatttcttggagaaaagcccagatcttcagtctctgagatatgctctcagtctttatacccaaactactgatgccttgataaagaaattcatagatactcaaacctcacagagtcgttcctccaaagatgccgtgggtcagatatctgttcatgtggacatcactgccaccccaggaacgggagatcataaagtcactgtaaaagtgattgctattaatgacctaaactggcagaccacagcaatgttccgcccctttgtggaagtttgtatactgggacccaaccttggagacaagaagagaaaacaaggcacaaaaacaaaaagcaacacatggtcaccaaagtacaatgaaacatttcagttcattctcggaaaggaaaatcggccaggggcttatgaacttcatctctcagttaaggattactgctttgccagagaagatcgaattatcggaatgacagtcattcagctacagaacatagcagaaaagggaagctatggggcatggtatcctcttctgaaaaatatctctatggatgaaactggtttgactatccttagaatactctctcagaggaccagtgatgatgtggctaaagaatttgtaagacttaaatctgaaacaagatctactgaagagagtgcttgaaacaaacactgcaagctaaatacataactataattgtttgactactgcatgcatgtgcaaatacatgggaatgtttagttcactacatttcaatgtttgccagtactcatgtacgatgtctacaaggtatgtaaaaaacctgctgaacttttataccaattctggtctttgggaaatcagtgttccatgaagtgccaaaattatgatgtaaagtgaaatatcaagaacaccttttaacatgtttattttgtttctttacccatttcacattcattaaacataatttttaaaaactagtcttttgagtttgcccatcagttggtctttgttaaatgagattatatggccctaggtcggggggcatttattactcgatttgcgatttagtgtgctaccgacatatggaagggctaccaatacccttttctccaaaaacgagattcgggtcactcgagaaagttttgttttttatggcgcaatttgggtttttgggaaaaaaaa

SEQ ID NO: 35 - DNA, 3285 bp, Homo sapiens
  misc_feature: Incyte ID No 7499693CB1
ggcggcgcagccggcacgcggcgctcgcgctccctccttaaatgagcctgggcgccccgcgcccgccacttcagtggatcccgcgccggggccgcgggcggagctgcctgccggtcccgcgccgcgcgtccgcactcctcggccctcgggcggtcgatgggacggggcgccgcggagcaggaggcggcgcccgtcggggtgctcgggccgcgcgggagcccactgtggggctcgggcatggcgggccgcaggacctgagctctcctcaggggagcggggaggcagctgctggccggcgatggggacggagtggggccgtcgccgccgcgccgagccgtgagcgccgagccaccgccgccgctacctcagcccttcgcgaagcgccgggcagctcgggaacatggccctggagcggctctgctcggtcctcaaagtgttgttaataacagtactggtagtggaagggattgccgtggcccaaaaaacccaagatggacaaaatattggaatcaagcatattcctgcaacccagtgtggcatttgggttcgaaccagcaatggaggtcattttgcttcgccaaattatcctgactcatatccaccaaacaaggagtgtatctacattttggaagctgctccacgtcaaagaatagagttgacctttgatgaacattattatatagaaccatcatttgagtgtcggtttgatcacttggaagttcgagatgggccatttggtttctctcctcttatagatcgttactgtggcgtgaaaagccctccattaattagatcaacagggagattcatgtggattaagtttagttctgatgaagagcttgaaggactgggatttcgagcaaaatattcatttattccagatccagactttacttacctaggaggtattttaaatcccattccagattgtcagttcgagctctcgggagctgatggaatagtgcgctctagtcaggtagaacaagaggagaaaacaaaaccaggccaagccgttgattgcatctggaccattaaagccactccaaaagctaagatttatttgaggttcctagattatcaaatggagcactcaaatgaatgcaagagaaacttcgttgcagtctatgatggaagcagttctattgaaaacctgaaggccaagttttgcagcactgtggccaatgatgtaatgcttaaaacaggaattggagtgattcgaatgtgggcagatgaaggtagtcggcttagcaggtttcgaatgctctttacttcctttgtggagcaaaagaaaaaagcaggagtatttgaacaaatcactaagactcatggaacaattattggcattacttcagggattgtcttggtccttctcattatttctattttagtacaagtgaaacagcctcgaaaaaaggtcatggcttgcaaaaccgcttttaataaaaccgggttccaagaagtgtttgatcctcctcattatgaactgttttcactaagggacaaagagatttctgcagacctggcagacttgtcggaagaattggacaactaccagaagatgcggcgctcctccaccgcctcccgctgcatccacgaccaccactgtgggtcgcaggcctccagcgtcaaacaaagcaggaccaacctcagttccatggaacttcctttccgaaatgactttgcacaaccacagccaatgaaaacatttaatagcaccttcaagaaaagtagttacactttcaaacagggacatgagtgccctgagcaggccctggaagaccgagtaatggaggagattccctgtgaaatttatgtcagggggcgagaagattctgcacaagcatccatatccattgacttctaatcttctgctaatggtgatgtgaattcttagggtgtgtacgtacgcagcctccagggcaccatactgtttccagcagccaacccttttctcccatcacaactacgaagaccttgatttaccgttaacctattgtatggtgatgtttttattctctcaggcagtctatatatgttaaaccaatcaaggaacttactctattcagtggaaacaataatcatctctattgcttggtgtcatttataggaagcactgccagttaaagagcattagaagaggtggttggatggagccaggctcaggctgcctcttcgttttagcaacaagaagactgctcttgactgataacagctctgtcaatattttgatgccacaataaacttgatttttctttacattccttttatttttcctttctctaaatttaatttgttttataagcctatcgttttaccatttcattttcttacataagtacaagtggttaatgtaccacatacttcagtataggcatttgttcttgagtgtgtcaaaatacagctagttactgtgccaattaagacccagttgtatttcacccatctgtttcttcttggctaatctctgtacttctgccttttaattactgggcccttattccttattttctgtgagaaataatagatgatatgatttattacctttcaattatatttttctcagttatactagaaaatttcataatcctgggatatatgtaccattgtcagctatgactaaaaatttgaaaaagataaaaatttctagcaagcctttgaagtttaccaagtatagtcacattcagtgacagcccattcattccagtaaagaatcatttcattcactttgggagaggcctataattacatttatttgcaatgtttctcttcgctagattgttacatagctcccattctgttggttttgcttacagcatatggtaaccaaggttagatgccagttaaaattccttagaaattggatgagccttgagattgcttcttaactgggacatgacatttttctagctcttatcaagaataacaacttccacttttttttaaactgcacttttgactttttttatggtataaaaacaataatttataaacataaaagctcattgtgttttttagacttttgatattatttgatactgtacaaactttattaaatcaagatgaaagacctacaggacagattcctttcagtgttcacatcagtggctttgtatgcaaatatgctgtgttggacctggacgctataacttattgtaaagaccttggaaatgtggacataagctctttctttccttttgttactgtatttagtttgtgataaattatctcactgggtgatatttatgcttctaaattaataccacaggtcccatatcatacatgcct

SEQ ID NO: 36 - DNA, 1825 bp, Homo sapiens
  misc_feature: Incyte ID No 2187465CB1
gcctgccgctgccttggctaccaggctcctcaggtggcagcgcttgcagtcgggctacggaggccgggttgccagattacgggaaagccatttaagaagttcctggaataatattagtcagagtaatataggatctgcaggaagtgtctcaagatagttggaaaagaagaatttctagactcttcatcaagatcttcatttatacagctgttaaatccaaggctactttggtgaaagcatgaataaaaatacatctactgtagtatcacccagtctacttgaaaaggatcctgcctttcagatgattacaattgccaaggaaacaggccttggcctgaaggtactaggaggaattaaccggaatgaaggcccattggtatatattcaggaaattattcctggaggagactgttataaggatggtcgtttgaagccaggagatcaacttgtctcagtcaacaaggaatctatgattggtgtatcatttgaagaagcaaaaagcataattaccagagccaagttgaggttagaatctgcttgggagatagcattcataagacaaaaatccgacaacattcagccagaaaatctgtcatgtacatcacttatagaagcttcaggagaatatggacctcaagcctcaacattaagtcttttttcttctcctcctgaaatactaatcccaaagacctcatccactcccaaaacaaataatgacattttatcttcttgtgagataaaaactggatacaacaaaacagtacagattccaattacttcagaaaacagtactgtgggtttgtctaatacagatgttgcttctgcctggactgaaaattatgggctacaagaaaagatctccctaaatccctctgttcgctttaaggcagagaaactggaaatggctctaaattatcttggtattcagcccacaaaggaacaacaccaagccctgagacagcaagtacaagcagactcaaaagggacagtgtcttttggagattttgtccaggttgccagaaacttgttttgcttgcagttggatgaagtaaatgttggtgcacatgaaatttccaatatattagattcacagcttcttccttgtgattcttcagaagcagatgaaatggaaaggctcaagtgtgaaagagatgatgccttgaaagaagtaaatacacttaaggaagccaaagctgtagttgaagaaacaagagccctgcgtagtcggattcatcttgctgaagctgctcagagacaggcacatggaatggaaatggactatgaagaagtgatccgtctgttagaggccaagattacagagctaaaggctcagcttgctgattattctgaccaaaataaagtaagcaaagcagtcatctcttccagttaccatggtttccttgccgttgtcatgtatcctgttttcattttcttttcatctgcacttctaaactaggtcagtgtttgtcttctattattcaatgataggatgctgtgtcctgatggggataatgtaaaggtcttgagccttcctttatccagatggctttgggatggaaaagcatgtgccccaaatttatttaggtcattggtcaaaattgttcagttcaggtattaagtcctggaactctttaacatttaattgattacattggtttttttcttttgtttctaaacctgctaatttgttttatgggaatgggagccaggggagtctaggaatgggtttgctctttgattagagcattcaaggaagtattaaagtgaacggaaggtggccggggatccactagttcaaccggcgcccccgtgctctctnnngccg

SEQ ID NO: 37 - DNA, 3214 bp, Homo sapiens
  misc_feature: Incyte ID No 3718011CB1
ggtcggtgggtgcctcggctcggctttccccggcgctggctgggctcagcggcccctgagcccaagcgacacacgccccgcggtccccgatccggcccctgggagagccgcgccgttctggaacccgggagcccccaacttcgcgccaagttcggagccgccttctgagggagacatgaaaaagatgagcaggaatgttttgctacaaatggaggaggaggaggacgacgacgatggggatatcgtgttggaaaaccttggacagacaattgtccccgatttgggatcactggaaagtcagcatgattttcgaaccccggagtttgaagaatttaatggaaaacctgactccctcttttttaatgatggccagcgaagaattgactttgttctagtatatgaggatgaaagcagaaaagagaccaataaaaagggtacaaatgaaaaacaaaggaggaaaagacaagcatacgaatctaaccttatctgtcatggcctgcagttagaagcaacaagatcagtattggatgacaagcttgtatttgtaaaagtacacgcaccatgggaggtgttatgtacgtatgctgagataatgcacatcaaattgcctctgaaacccaatgatctgaaaaaccggtcctcagcctttggtacactcaactggtttaccaaagtcctcagtgtagacgaaagcatcatcaagccagagcaagagtttttcactgccccatttgagaagaaccggatgaatgatttttacatagttgatagagatgctttcttcaatccagccaccagaagccgcattgtttacttcatcctctctcgggtcaagtatcaagtgataaacaatgttagcaagtttgggatcaacagacttgtaaactctgggatctacaaggcagctttcccactccatgattgcaaattccgccgtcagtcagaggatcccagctgccctaatgaacggtaccttctgtacagagaatgggctcatcctcgaagcatatacaaaaagcagcccttggatcttatcaggaaatactatggagagaagattggaatctactttgcttggctgggctattacactcagatgcttctcctggccgcagttgtaggagtggcttgctttctctatggatatcttaatcaagataactgtacatggagcaaagaagtttgtcatcctgatattggtggcaagatcataatgtgtcctcagtgtgataggctttgtccattctggaaactcaatattacttgcgagtcctcaaagaaattgtgcatcttcgacagttttggaaccctggtctttgcagtatttatgggagtatgggttaccttgtttttggagttttggaagcgacgccaggcagaacttgagtatgaatgggatactgttgagttacagcaggaagaacaagcccgaccagaatacgaagcacgatgtactcacgtagtgataaatgagattactcaggaagaagaacgcattccctttactgcctggggaaaatgtatacggataaccctctgtgccagtgctgtctttttctggatcctattgatcatcgcttcagttattgggatcattgtctataggctctcggtgttcattgtattttctgcaaaacttcccaagaacattaatggaacagacccaatccagaaatacctgactccacagacagccacgtccatcacggcctccatcatcagctttataattatcatgattctgaacaccatatatgaaaaagtggcaattatgattactaacttcgaactcccaaggacccagactgattatgagaacagcctcaccatgaagatgttcttattccagtttgtcaactactactcttcatgcttctacatagcattctttaagggcaaatttgtaggctatccaggagacccagtttattggttgggaaaatacagaaatgaagagtgtgacccaggtggctgtcttcttgaactgacaactcagctgacaataatcatgggaggaaaagcaatctggaataacatacaagaagtattattgccctggatcatgaatctaattgggcgatttcacagagtttctggatcagaaaagataaccccacgatgggaacaggactaccatctgcagcctatgggcaaactgggattattttatgaatatcttgaaatgattattcagtttgggttcgtcaccttatttgtggcctcttttccactggcccctctgttggctctcgtgaacaatatattggaaataagagtggacgcatggaaactgaccacccagtttagacgcctggtaccagagaaagcccaagacattggagcatggcagcccatcatgcaaggaatagcaattctggctgtggtgaccaatgccatgatcatagctttcacgtcggacatgatcccccgcctagtgtactactggtccttctccgtccctccctacggggaccacacttcctacaccatggaagggtacatcaacaacactctctccatcttcaaagtcgcagacttcaaaaacaaaagcaagggaaacccgtactctgacctgggtaaccataccacatgcaggtatcgtgatttccgatacccacctggacacccccaggagtataaacacaacatctactattggcatgtgattgcagccaagctggcttttatcattgtcatggagcacgtcatctactctgtgaaatttttcatttcatatgcaattcccgatgtatcaaaacgcacaaagagcaagatccagagagaaaaatacctaacccaaaagcttcttcatgagaatcacctcaaagatatgacgaaaaatatgggggtgatagctgagcggatgatagaagcagtagataacaatttacggccaaaatcagaataagagctttatgttctgagaagcactttaaggaatttagctttgtcaaaatatattaggaatcactaatgagaatgtgtaagttaaatcactttggcaaatatgagtctcaactattgccatttcctcatgtattatttttcagtttcagctagcgatgcagaaactggaaaatgtaaaacttagatcatgaagggcataaaacttatcacccggaaaactcaatgttactttttctgataattgggattttacagaaaagtcctcagtgtgttaaaaccacccttctaagtagatggatcttttttc

SEQ ID NO: 38 - DNA, 1597 bp, Homo sapiens
  misc_feature: Incyte ID No 7500509CB1
gagagggctgagggagcagggttgagcaactggtgcagacagcctagctggactttgggtgaggcggttcagccatgaggctggctgtgcttttctcgggggccctgctggggctactggcagagagcactggaacaaccagccacaggactaccaagagccacaaaaccaccactcacaggacaaccaccacaggcaccaccagccacggacccacgactgccactcacaaccccaccaccaccagccatggaaacgtcacagttcatccaacaagcaatagcactgccaccagccagggaccctcaactgccactcacagtcctgccaccactagtcatggaaatgccacggttcatccaacaagcaacagcactgccaccagcccaggattcaccagttctgcccacccagaaccacctccaccctctccgagtcctagcccaacctccaaggagaccattggagactacacgtggaccaatggttcccagccctgtgtccacctccaagcccagattcagattcgagtcatgtacacaacccagggtggaggagaggcctggggcatctctgtactgaaccccaacaaaaccaaggtccagggaagctgtgagggtgcccatccccacctgcttctctcattcccctatggacacctcagctttggattcatgcaggacctccagcagaaggttgtctacctgagctacatggcggtggagtacaatgtgtccttcccccacgcagcacagtggacattctcggctcagaatgcatcccttcgagatctccaagcacccctggggcagagcttcagttgcagcaactcgagcatcattctttcaccagctgtccacctcgacctgctctccctgaggctccaggctgctcagctgccccacacaggggtctttgggcaaagtttctcctgccccagtgaccggtccatcttgctgcctctcatcatcggcctgatccttcttggcctcctcgccctggtgcttattgctttctgcatcatccggagacgcccatccgcctaccaggccctctgagcatttgcttcaaaccccagggcactgagggggttggggtgtggtgggggggtacccttatttcctcgacacgcaactggctcaaagacaatgttattttccttccctttcttgaagaacaaaaagaaagccgggcatgacggctcatgcctgtaatcccagcactttgggaggctgaggcaggtggatcactggaggtcaggagtttgagaccagcctggccaacatggtgaaaccctgtctctactaaaaatacaattagccaggtgtggcggcgtaatcccagctggcctgtaatcccagctacttgggaggctgaggcagaactgcttgaacccaggaggtggaggttgcagtgagccgtcatcgcgccactaagccaagatcgcgccactgcactccagcctgggcgacagagccagactgtctcaaataaataaatatgagataatgcagtcgggagaagggagggagagaattttattaaatgtgacgaactgccccccccccacccccccagcaggagagcagcacgaccg

SEQ ID NO: 39 - DNA, 1923 bp, Homo sapiens
  misc_feature: Incyte ID No 7497865CB1
ctcagctcctgcctctcactccctctctatctgccttctgtttctctttgggtctctcctgctctcctctttctggcctgcctcctctctctaatcctgcctctcttcctctcccccccttgccttgccccctctcactctaggccctcagctccagcctctggccctgacctcgagctgtgtcctgattctgtctctgccccaggactgcagggctccaggaggtctgggctgcctccagcttcccactcccaggttgcggctggactgggactggttcctttccagttgaatctggcagccaaacctctcctccccctcacctgacaggtgcagcggcctggctggggagcccgcccgccggccggccagggatggaagcgacaggaatctcattagcatctcaattaaaggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggcagggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgacggagtctgagaaaaggcagaagaaggggggcacaagggcaccttctcccttgaggctgccctgcccacgtgggattcacaggggcctgagtagggcccggggaagcagagccctaagggattaaggctcagacacctctgagagcaggtgggcactggctgggtacggtgccctccacaggactctccctactgcctgagcaaacctgaggcctcccggcagacccacccacccctggggctgctcagcctcaggcagggggatccactagttcttaagcgccgcaccgcgtggcca

SEQ ID NO: 40 - DNA, 3025 bp, Homo sapiens
  misc_feature: Incyte ID No 3116578CB1
gggcggccgagcgggcggcgggcatgagcggggcgggcagggcgctggccgcgctgctgctggccgcgtccgtgctgagcgccgcgctgctggcccccggcggctcttcggggcgcgatgcccaggccgcgccgccacgagacttagacaaaaaaagacatgcagagctgaagatggatcaggctttgctactcatccataatgaacttctctggaccaacttgaccgtctactggaaatctgaatgctgttatcactgcttgtttcaggttctggtaaacgttcctcagagtccaaaagcagggaagcctagtgctgcagctgcctctgtcagcacccagcacggatctatcctgcagctgaacgacaccttggaagagaaagaagtttgtaggttggaatacagatttggagaatttggaaactattctctcttggtaaagaacatccataatggagttagtgaaattgcctgtgacctggctgtgaacgaggatccagttgatagtaaccttcctgtgagcattgcattccttattggtcttgctgtcatcattgtgatatcctttctgaggctcttgttgagtttggatgactttaacaattggatttctaaagccataagttctcgagaaactgatcgcctcatcaattctgagctgggatctcccagcaggacagaccctctcgatggtgatgttcagccagcaacgtggcgtctatctgccctgccgccccgcctccgcagcgtggacaccttcagggggattgctcttatactcatggtctttgtcaattatggaggaggaaaatattggtacttcaaacatgcaagttggaatgggctgacagtggctgacctcgtgttcccgtggtttgtatttattatgggatcttccatttttctatcgatgacttctatactgcaacgggggtgttcaaaattcagattgctggggaagattgcatggaggagtttcctgttaatctgcataggaattatcattgtgaatcccaattattgccttggtccattgtcttgggacaaggtgcgcattcctggtgtgctgcagcgattgggagtgacatactttgtggttgctgtgttggagctcctctttgctaaacctgtgcctgaacattgtgcctcggagaggagctgcctttctcttcgagacatcacgtccagctggccccagtggctgctcatcctggtgctggaaggcctgtggctgggcttgacattcctcctgccagtccctgggtgccctactggttatcttggtcctgggggcattggagattttggcaagtatccaaattgcactggaggagctgcaggctacatcgaccgcctgctgctgggagacgatcacctttaccagcacccatcttctgctgtactttaccacaccgaggtggcctatgaccccgagggcatcctgggcaccatcaactccatcgtgatggcctttttaggagttcaggcaggaaaaatactattgtattacaaggctcggaccaaagacatcctgattcgattcactgcttggtgttgtattcttgggctcatttctgttgctctgacgaaggtttctgaaaatgaaggctttattccagtaaacaaaaatctctggtccctttcgtatgtcactacgctcagttcttttgccttcttcatcctgctggtcctgtacccagttgtggatgtgaaggggctgtggacaggaaccccattcttttatccaggaatgaattccattctggtatacgtcggccacgaggtgtttgagaactacttcccctttcagtggaagctgaaggacaaccagtcccacaaggagcacctgactcagaacatcgtcgccactgccctctgggtgctcattgcctacatcctctatagaaagaagattttttggaaaatctgatggctcccactgagatgtgctgctggaagactctagtaggcctgcagggaggactgaagcagcctttgttaaagggaagcattcattaggaaattgactggctgcgtgtttacagactctgggggaagacactgatgtcctcaaactggttaactgtgacacggctcgccagaactctgcctgtctatttgtgacttacagatttgaaatgtaattgtcttttttcctccatcttctgtggaaatggatgtctttggaacttcattccgaggagataagctttaactttccaaaagggaattgccatgggtgtttttcttctgtggtgagtgaaacaatctgaggtctggttcttgctgaccttgttgccctgcaaacttcctttccacgtgtacgcgcacaccaacacgaaatgccatcactcctactgcggctgctatgaagcttactggttgtgatgtgttataatttagtctgtttttttgattgaatgcagtttaatgtttccagaaagccaaagtaattttcttttcagatatgcaaggcttggtgggtccaaaaaatgtctatcacaagccattttttccttttcctctctcgaaaagttaaaatatctatgtgttattcccaaaccctcttacctatgtatctgcctgtctgtccatcatcttccttcctccctatctctgtgtatctggatggcagccgctgcccaggggagtggctgtggggagggcaggtactgtctttgcctgtgggtccagctgagccatccctgctgggtgatgctgggcaagacccttggcccgtctgggccttggcttcctcacttgtgaaatgagcgggaagatgactctcagttccttccacctcttagacatggtgaggtaacagacatcaaaagcttttctgaaatcttcagaagaaatagttccattacagaaaactcttcaaaataaatagtagtgaaaacttttaaaaactctcattggagtaagtcttttcaagatgatcctccacaatggaggcagcgttcctacttgtcatcacacagctgaagacattgtttcttaggtgtgaaatcggggacaaag

SEQ ID NO: 41 - DNA, 1870 bp, Homo sapiens
  misc_feature: Incyte ID No 2797803CB1
atgcctgcgcgcagtcgccaccgcccccgcctccactccggctccccgccccgggctccgcccccgccgcttgaggcgcttcactccggcgaggcggggagggccccggactccgacggcggctcggacgccgactcggaggtgggtccggggagcccgactcggaccgcggaggcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacagcgttcctagaatcccatgggcctttgccatgacggaagtatgtggcatgattctgtgctatatttggctcctggttcttcttcttcacaagcacaggtcaatacttctgcgaaggctctgtagtctgatgggaactgtattcttgcttcgctgctttaccatgtttgtgacctccctctccgtgccaggacaacacctgcagtgtactggaaagatatatggcagtgtatgggagaaattacatcgagcctttgccatttggagtggctttggtatgaccctgactggcgttcacacatgtggagattacatgtttagtggccacacagtcgtcctaactatgctgaatttctttgtcaccgaatatacaccaagaagctggaatttcttgcacactttatcctgggttctcaacctctttggaatcttcttcatcttggctgcccatgaacattattctattgatgtgtttattgctttttatataacaacaagactctttttgtactaccatactctggccaataccagagcatatcagcagagtaggagagcaaggatttggtttcccatgttctctttttttgaatgcaatgttaatggcacagtacctaatgaatattgttggccattttcaaaaccagcaataatgaaaagactaattggatgaatactatctttctaatgaatttgtgattaaatatataatagttgttgaaaatgagtaactttgcgttctccccctaggttgttcttagatgcctggcttatgtgttgacaaagtaaagttttctgttctgagcaacagttatgattataaacacagcaagaaagaacaatcaagagtcttatgtagctatttgaacagaaagcttaagtagatgttttctgccccattctctttaggaagacttaatgtggtgattgaagtcaggctgtacccttacctgtggagtatttgcttatggaactttaaacaagtcaacttgagcagtttgctggttgaggaattttcattgatttccagtagggctctagtcaagaaataatatgttttgaggctccttattacctttagaagaagaaaccttacaagtgcagta

SEQ ID NO: 42 - DNA, 2628 bp, Homo sapiens
  misc_feature: Incyte ID No 5433453CB1
ggggaggggaggggccgggccgggccgggcgggaggagccgctcgccggttttgccgcctccgcctttgccttcgcagccgcctccagggcaatttgcatatttctccaaagaaccatccagaacctgagcagcctgtcttcagacagagagaggcccacggctgtttcttgaaatctggcgctgggaatggccatgtggaacaggccatgccagaggctgcctcagcagcctctggtagctgagcccactgcagagggggagccacacctgcccacgggccgggagctgactgaggccaaccgcttcgcctatgctgccctctgtggcatctccctgtcccagttatttcctgaacccgaacacagctccttctgcacagagttcatggcaggcctggtgcagtggctggagttgtctgaagctgtcttgccaaccatgactgcttttgcgagcggcctgggaggtgaaggagcagatgtgtttgttcaaattttactgaaggaccccatcttgaaggacgacccgacggtgatcactcaggaccttctgagcttctcactcaaggatgggcactatgacgcccgggccagagtcctcgtttgccacatgacctccctgctccaagtgcccttggaggagctggatgtccttgaagagatgttcctggagagcctgaaggaaatcaaagaagaggaatctgaaatggccgaggcatcccgaaagaagaaagaaaaccggaggaaatggaagcgttatctcctgataggcctggcgactgtcggaggcggaacggtgatcggtgtgactggaggtctagctgcaccccttgttgccgctggagcagcgacgattattggcagcgccggggcagcggctctgggctcagcagccggcatagccatcatgacctcgctgtttggtgcagctggagctggcctgacaggatacaagatgaagaagcgagtgggagccattgaagagttcacgtttctgcctctgacggagggcaggcagctgcacatcaccatcgccgtcacggggtggctcgcttctggcaaataccgcaccttcagtgccccgtgggctgccctggcccacagccgtgagcagtactgcctggcctgggaagccaagtacctgatggagctcggcaatgccctggagaccatcctcagtggtctcgccaacatggtggcccaggaggccctaaagtacacagtgttgtctggcattgtggctgccctgacctggccagcctcactcctcagtgtcgccaatgtcatcgacaacccctggggggtgtgtctccatcgatcagcagaggttggcaagcacctggcccacatcctgctctcccggcagcaggggcgacgacctgtcaccttgattggcttcagcctgggagccagagtcatctacttctgtctgcaggagatggctcaagagaaagattgccaaggaatcatcgaggacgtcatcctgctgggtgcgcctgtggagggagaagccaagcattgggagcctttccggaaggtggtgtccgggaggatcatcaacggctactgcaggggagactggctgctgagtttcgtgtaccgcacatcctcggtgcagctccacgtcgccggcctacagcccgtgctgctgcaggacaggagggtggagaacgtggacctgacctctgtggtcagcggccacctggactatgccaagcagatggatgccatcctgaaggccgtgggcatccgcaccaagccaggctgggacgagaaggggctcttgctggccccaggctgcctgccctccgaggagcctcgccaggcagcagctgccgcctcatcaggcgagaccccccaccaggttgggcaaacccagggtcccatatccggagacacctccaaattggccatgtccacagaccccagccaagcccaggtgccagtagggctggaccagtctgaaggggcctcccttcctgctgctgccagccctgaaaggccccccatctgcagccatggcatggaccccaacccactgggctgccccgattgtgcctgcaagacccagggccccagcacggggctggactgaccacagcaggggacctgagccgtcttccccagtctccatatgcagctctctcttataccctcgggttcctcccaggagctctggaggtacaggatttccacaggcctctttcctaaatggaaggaattggaactgaaagggaaaggaaatggaaggaaggggaatttggaggagagaacacgcccacccttgggaagctgcctgtccccagaggagccccaccagggagcagctgccccctcatcagagacctgcagagtcaaccaagcacaggttagagtcccaggaccggaaaccaactgtgggctttctgtacttctcatagctttggagtctggctgtccatcaggaggtcccgagggctctctggggcctgaggctcccacaccagctctcccctggcctcaataaaaccaggtgcatgcctgttcttccatccacactccagggctgcccaccagctgacaggcaccatcaactggcagcaacagagcaggcgcaggtacaaagaaggcagctcactcctgctcttagg

SEQ ID NO: 43 - DNA, 694 bp, Homo sapiens
  misc_feature: Incyte ID No 6246071CB1
gcgatctagaaccttggatctgcctgccaggccatcctgggcgctgcaggaagcaacatgacttaggtaactgcccagaggtgcaccagacatgatgcagcagccgcgagtggagacagataccatcggggctggcgaggggccacagcaggcagtgccctggtcagcctgggtcacgaggcatggctgggtgcgctggtgggtgagccacatgcccccgagctggatccagtggtggagcacctcgaactggcggcaaccgctgcagcgcctgctgtggggtctggaggggatactctacctgctgctggcactgatgttgtgccatgcactcttcaccactggctcccacctgctgagctccttgtggcctgtcgtggccgcggtgtggcgccacctgctaccggctctcctgctgctggtgctcagtgctctgcctgccctcctcttcacggcctccttcctgctgctcttctccacactgctgagccttgtgggcctcctcacctccatgactcacccaggcgacactcaggatttggatcaatagaagggcaaccccatcccactgcctgtgtctgttgagccctggcctagggcctgagaccccacggggagagggagggcaatgggatcagggctccctgccttggcagggcccagacccctagtccctaacaggtagactggcctg

SEQ ID NO: 44 - DNA, 1359 bp, Homo sapiens
  misc_feature: Incyte ID No 7500557CB1
atgcctgcgcgcagtcgccaccgcccccgcctccactccggctccccgccccgggctccgcccccgccgcttgaggcgcttcactccggcgaggcggggagggccccggactccgacggcggctcggacgccgactcggaggtgggtccggggagcccgactcggaccgcggaggcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacagcgttcctagaatcccatgggcctttgccatgacggaagtatgtggcatgattctgtgctatatttggctcctggttcttcttcttcacaagcacagatatatggcagtgtatgggagaaattacatcgagcctttgccatttggagtggctttggtatgaccctgactggcgttcacacatgtggagattacatgtttagtggccacacagtcgtcctaactatgctgaatttctttgtcaccgaatatacaccaagaagctggaatttcttgcacactttatcctgggttctcaacctctttggaatcttcttcatcttggctgcccatgaacattattctattgatgtgtttattgctttttatataacaacaagactctttttgtactaccatactctggccaataccagagcatatcagcagagtaggagagcaaggatttggtttcccatgttctctttttttgaatgcaatgttaatggcacagtacctaatgaatattgttggccattttcaaaaccagcaataatgaaaagactaattggatgaatactatctttctaatgaatttgtgattaaatatataata

SEQ ID NO: 45 - DNA, 1585 bp, Homo sapiens
  misc_feature: Incyte ID No 6978182CB1
gctggcgagcccggaacgcctctggtcacagctcagcgtccgcggagccgggcggcgctgcagctgcacttggctcgtctgtgggtctgacagtcccagctctgcgcggggaacagcggcccggagctgggtgtgggaggaccaggctgccccaagagcgcggagactcacgcccgctcctctcctgttgcgaccgggagccgggtaggaggcaggcgcgctccctgcggccccgggatgacttctcagcgttcccctctggcgcctttgctgctcctctctctgcacggtgttgcagcatccctggaagtgtcagagagccctgggagtatccaggtggcccggggtcagacagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagattcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtataggacatgaggaaatgttgtgttcagaaatgaataaatggaatgccctcaaaaa

SEQ ID NO: 46 - DNA, 1495 bp, Homo sapiens
  misc_feature: Incyte ID No 1985321CB1
ctgcaagctataagctctgcaagtggtgaccccgacgtgatcgccttgaagttacgcttgaaggaggaaaactcatcaattttcggggaatcccgttcatcatctccggatccctctcagtggcagccgagaagaaccacaccagttgcctggtgaggagcagcctgggcaccaacatcctcagcgtcatggcggcctttgctgggacagccattctgctcatggattttggtgttaccaaccgggatgtggacaggggctatctggccgtgcttactatcttcactgtcctggagttcttcacagcggtcattgccatgcacttcgggtgccaagccatccatgcccaggccagtgcacctgtgatcttcctgccaaacgccttcagcgcagacttcaacatccccagcccggcagcctctgcgccccctgcctatgacaatgtggcatatgcccaaggagtcgtctgagtagcagatgtggcacctgcgggtggagtccagccttttccctctgggcccagcctctccccacccccaccttgttcatcaggggccagccccatcccagctgccctccctcaccacatctacacatactccggcatctgagtgaagtgtccccagggacatctctcccacactttccgcagtgctttctttctaaaagacaccgggctgacgtcaggggtgtgtgtccttcagctccctgagccctgtcacccttccaggacacccaccttgtgcatctaagcatttctctgctcattggggaaatcctggcctcattggagactcaggttcgaggcctgccctgaccctcgggcctcgggaaggtcagagagcccggaatcctccagaatggaagagtctgactctggcattccacagaggtgccgataccaggccaaggcctcacagcagggtagtggcctggccgcaggtctcctggccccaagatcagctctgtcctttgtcatctgttgccacatccatggaactcaggtttcctatttggaaactagagtgttgaaccagataaggttcatcaggcccttccagctccccaggctccctgaagtcctgggtctaggccaggcattgtccccctgcttcctggaaaccctcattttccttgtctgtaatatgaagtcagcattggccccgccccccaccccctaccatctcccagtggaggggaggttgcaggggagagctgccgccagcccactcctgaggcaccaccacagtcagcatcgacaggggcacagcagtggcagtttgggacctccctgtgcctctcagcactcccttccccacccccatagcccaaggacaaggctaccacagaaggttaccacaggacctgggcttcgtctccaggggacaaggagacactgtcagcctggtgttcaccaggcctggtagatgagatggcttgtctcatccacaccacagaaggaaataaaccatgtggcttaaaaaaaaaaaaaaaa